(19) United States
(12) Patent Application Publication
(10) Pub. No.: US 2019/0023809 A1
(43) Pub. Date: Jan. 24, 2019

(54) ANTI-NICOTINAMIDE PHOSPHORIBOSYLTRANSFERASE ANTIBODY GENES AND METHODS OF USE THEREOF

(71) Applicant: The Children's Mercy Hospital, Kansas City, MO (US)

(72) Inventors: Shui Qing Ye, Kansas City, MO (US); Daniel P. Heruth, Kansas City, MO (US); Li Qin Zhang, Kansas City, MO (US)

(21) Appl. No.: 15/756,198

(22) PCT Filed: Sep. 6, 2016
(86) PCT No.: PCT/US16/50444

§ 371 Date: Feb. 28, 2018

Related U.S. Application Data
(60) Provisional application No. 62/213,957, filed on Sep. 3, 2015.

(51) Int. Cl. C07K 16/40; A61K 38/19; C12N 15/62; C12N 15/113

(52) U.S. Cl. CPC C07K 16/40; A61K 38/191; C12N 15/62; A61P 11/00; C07K 2317/30; C07K 2317/622; C07K 2319/21; C12N 15/113

(57) ABSTRACT

The present disclosure provides anti-NAMPT cDNA clones and the amino acid sequences encoded by the clones. Such clones and amino acid sequences are combinable in several variations and can be used to decrease NAD synthesis in a targeted cell population.

### SEQUENCE LISTING

This application contains a sequence listing in paper format and in computer readable format, the teachings and content of which are hereby incorporated by reference.

### FIELD

The present application relates to cDNA clones of anti-nicotinamide phosphoribosyltransferase antibody (anti-NAMPT) genes. More particularly, this disclosure relates to cDNA clones of anti-NAMPT genes encoding monomers of the single chain variable fragment (scFv) formed from both the heavy (V_{H}) and light (V_{L}) chains of immunoglobulin's and the construction of antibodies using such clones.

### BACKGROUND

NAMPT is a pleiotropic protein, originally named pre-B-cell colony enhancing factor (PBEF) after its function to promote pre-B-cell colony formation. NAMPT is the rate-limiting enzyme in the salvage pathway of mammalian NAD biosynthesis that catalyzes the condensation of nicotinamide with 5-phosphoribosyl-1-pyrophosphate to yield nicotinamide mononucleotide, an intermediate in the biosynthesis of NAD. NAMPT was also formerly known as VISFATIN, an adipokine produced by visceral fat that mimics the effects of insulin. NAMPT is a pleiotropic protein with functions in innate immunity, inflammation, apoptosis and oxidative stress, etc. The gene that codes for NAMPT is an essential one, thought to be critical for survival. Homozygous NAMPT knockout mice are embryonically lethal and adult mice with Tamoxifen induced Cre deletion of its two copies die within a few days.

Because of its pleiotropic and essential functions, dysregulation of the NAMPT gene has been implicated in the susceptibility and pathogenesis of several human diseases and conditions such as acute respiratory distress syndrome, arthritis, cancer, coronary artery disease, and diabetes. In particular, studies have demonstrated that NAMPT is overexpressed in neutrophils of both patient and animal models of acute lung injury (ALI) and acute respiratory distress syndrome (ARDS). In addition, a multitude of experimental and clinical data point to the causative role of neutrophils in lung injury. Activation and transmigration of neutrophils is a hallmark event in the progression of ALI and ARDS. Since NAMPT has an antiapoptotic role, it functions to prolong neutrophil presence at the site of inflammation, and hence results in hyperinflammatory tissue damage because of the neutrophil's capacity for the production of toxic mediators.

Studies have also demonstrated that NAMPT mediates inflammatory response and tissue destruction. NAMPT is known to be up-regulated in both Juvenile idiopathic arthritis (JIA) and rheumatoid arthritis (RA) and is a key biomarker in arthritis. Knockdown of NAMPT in RAW 264.7 macrophage has been shown to attenuate their differentiation into osteoclasts. Knockdown of NAMPT has also been shown to significantly attenuate the immune response and bone erosion in mice having collagen-induced arthritis. NAMPT is believed to mediate inflammatory response and tissue destruction in multiple cell types, including synovial fibroblasts, macrophages and neutrophils.

Pulmonary surfactant is a complex mixture of phospholipids, lipids, and proteins that lines the alveolar regions of the lungs, thereby stabilizing the surface of the air-blood barrier and improving gas exchange. Surfactant protein B (SP-B) is a critical component of pulmonary surfactant which is secreted by two types of lung epithelial cells, the alveolar type II cell and club cell. SP-B is the only surfactant protein strictly required for breathing, its absence is associated with a lethal respiratory failure in mice and humans. TNF-α inhibits expression of pulmonary surfactant protein in epithelial cells. The decrease in SP-B protein concentration is considered to contribute to the severity of lung inflammation and injury following infection. On the other hand, anti-inflammatory properties as well as protection from oxygen-induced and endotoxin (LPS)-induced lung injuries also have been described for SP-B.

ALI and ARDS are common syndromes with a high mortality rate and characterized by pulmonary inflammation. Abnormal surfactant function is thought to play a central role in the evolution of ALI/ARDS. SP-B was low in the bronchoalvelolar lavage (BAL) of patients at risk for ARDS before the onset of clinically defined lung injury, and in patients with established ARDS. Although SP-B involvement and down-regulation in inflammatory processes has been generally recognized, the exact mechanism behind that has not yet been elucidated.

NAMPT is a novel biomarker in ARDS with genetic variants conferring ARDS susceptibility. In vivo, overexpression of NAMPT aggravated ALI and IL-1β or TNF-α mediated cell permeability and IL-8 secretion in epithelial cells and endothelial cells, while knockdown of NAMPT expression attenuated ventilator induced lung injury (VILI) and IL-1β or TNF-α mediated cell permeability and IL-8 secretion. These findings indicate that NAMPT regulates epithelial functions upon ALI.

Because NAMPT is an essential gene, it is not feasible to undertake ubiquitous knockdown of NAMPT without affecting its normal physiological function in other organs of the body. However, knockdown of NAMPT in neutrophils would enhance neutrophil apoptosis and shorten the life of neutrophils, which could ameliorate long lasting neutrophil-related inflammatory damage. Accordingly, there is a need for the ability to knockdown NAMPT in specific cell types, rather than in an entire organ or throughout the body. In particular, there is a need for a neutrophil-specific NAMPT gene knockdown, as well as a fibroblast specific NAMPT gene knockdown, as well as a macrophage specific NAMPT gene knockdown.

### SUMMARY

The present disclosure broadly concerns two unique anti-NAMPT antibody genes. Each of the genes encodes a single-chain fragment variable (scFv) antibody, designated scFv1 or scFv2. Each of these antibody fragments is a fusion protein of the variable regions of the heavy (V_{H}) and light (V_{L}) regions or chains of immunoglobulins connected with a shorter linker peptide of from about ten to about 25 amino acids. These two genes can be engineered to express NAMPT-targeted therapy and any cell-targeted therapy. ScFv1 has an exemplary nucleotide sequence identified as SEQ ID NO 1, and scFv1 has an exemplary amino acid sequence identified as SEQ ID NO 2. ScFv2 has an exemplary nucleotide sequence identified as SEQ ID NO 3, and scFv2 has an exemplary amino acid sequence identified as SEQ ID NO 4.

The disclosure also concerns cDNA clones of anti-NAMPT antibody genes. The clones encode monobodies or monomers including the variable regions from the V_{H} chain of immunoglobulin linked with the variable regions from the V_{L} chain of immunoglobulin connected by a linker. Thus, one cDNA clone of an anti-NAMPT antibody gene encodes a monomer scFv1 having linked variable regions from the V_{H} and V_{L} chains. Another cDNA clone of an anti-NAMPT antibody gene encodes a monomer scFv2 having linked variable regions from the V_{H} and V_{L} chains.

The disclosure further concerns cDNA clones of anti-NAMPT antibody genes encoding dibodies or dimers including two monomers, each monomer including the variable regions from the V_{H} chain of immunoglobulin linked with the variable regions from the V_{L} chain of immunoglobulin, the monomers being connected by a linker.

The disclosure further concerns cDNA clones of anti-NAMPT antibody genes encoding dimers formed of a scFv1 connected with a scFv1 by a linker.

The disclosure further concerns cDNA clones of anti-NAMPT genes encoding dimers formed of a scFv1 connected with a scFv2 by a linker. In one non-limiting example, a dimer of the present disclosure has a nucleotide sequence identified as SEQ ID NO 5, and an amino acid sequence identified as SEQ ID NO 6.

Another non-limiting example of a dimer of the present disclosure provides cDNA clones of anti-NAMPT genes encoding dimers formed of a scFv2 connected with another scFv2 by a linker.

In a further aspect of the present disclosure, cDNA clones of anti-NAMPT genes encoding tribodies or trimers including three monomers are provided, each monomer including the variable regions from the V_{H} chain of immunoglobulin linked with the variable regions from the V_{L} chain of immunoglobulin, the monomers being connected by linkers.

Non-limiting examples of a tribodies or trimers of the present disclosure include cDNA clones of anti-NAMPT antibody genes encoding trimers formed of three linked scFv1 monomers, or three linked scFV2 monomers, where the monomers are each connected by linkers, which may be the same linker or each linker may be distinct.

Additional non-limiting examples of a tribodies or trimers of the present disclosure include cDNA clones of anti-NAMPT antibody genes encoding trimers formed of two scFv1 monomers and one scFv2 monomer, where the monomers are each connected by linkers, which may be the same linker or each linker may be distinct.

Additional non-limiting examples of a tribodies or trimers of the present disclosure include cDNA clones of anti-NAMPT antibody genes encoding trimers formed of one scFv1 monomer and two scFv2 monomers, where the monomers are each connected by linkers, which may be the same linker or each linker may be distinct.

Another aspect of the present disclosure concerns cDNA clones of anti-NAMPT genes encoding tetrabodies or tetramers including four monomers, each monomer including the variable regions from the V_{H} chain of immunoglobulin linked with the variable regions from the V_{L} chain of immunoglobulin, the monomers being connected by linkers. Further aspects of the present disclosure can include five monomers linked together by linkers, six monomers, seven monomers, eight monomers, nine monomers, or ten monomers, where each monomer is connected to at least one other monomer by a linker, where the linker may be the same linker or each linker may be distinct.

Non-limiting examples of tetrabodies or tetramers of the present disclosure include cDNA clones of anti-NAMPT antibody genes encoding tetramers formed of four scFv1 monomers, the monomers connected by linkers, where the monomers may be connected by the same linker or each linker may be distinct.

In another non-limiting examples of tetrabodies or tetramers of the present disclosure include cDNA clones of anti-NAMPT antibody genes encoding tetramers formed of four scFv2 monomers, the monomers connected by linkers, where the monomers may be connected by the same linker or each linker may be distinct.

Further non-limiting examples of tetrabodies or tetramers of the present disclosure include cDNA clones of anti-NAMPT antibody genes encoding tetramers formed of three scFv1 monomers, and one scFv2 monomer, with each monomer being connected to at least one other monomer by a linker, where the monomers may be connected by the same linker or each linker may be distinct.

Additional non-limiting examples of tetrabodies or tetramers of the present disclosure include cDNA clones of anti-NAMPT antibody genes encoding tetramers formed of two scFv1 monomers, and two scFv2 monomers, the monomers being connected by linkers, where the monomers may be connected by the same linker or each linker may be distinct. In one embodiment, such a tetrabody or tetramer may be formed by two dimers, where each dimer has a nucleotide sequence identified as SEQ ID NO 7, and an amino acid sequence identified as SEQ ID NO 8. In such an embodiment, the dimers would be connected by a linker.

Further non-limiting examples of tetrabodies or tetramers of the present disclosure include cDNA clones of anti-NAMPT antibody genes encoding tetramers formed of one scFv1 monomer, and three scFv2 monomers, the monomers connected by linkers where the monomers may be connected by the same linker or each linker may be distinct.

Another aspect of the present disclosure concerns the variable regions of the heavy (V_{H}) and light (V_{L}) chains of immunoglobulins connected with a shorter linker peptide to form the fusion protein that comprises the scFv1 and scFv2 monomers. As previously described, each scFv1 or scFv2 monomer includes a V_{H} chain and a V_{L} chain. A scFv1 V_{H} chain may be substituted for a scFv2 V_{H} chain, and a scFv1 V_{L} chain may be substituted for a scFv2 V_{L} chain. These heavy (V_{H}) and light (V_{L}) chains may be substituted in any monomer described herein, including those monomers that are part of dimers, trimmers, tetramers, and the like.

Non-limiting examples of monomers of the present disclosure include cDNA clones of anti-NAMPT antibody genes encoding monomers formed of a scFv1 V_{H} chain and a scFv2 V_{L} chain, the chains connected by a linker. Such a monomer may be part of a dimer, trimer, or tetramer as described herein.

A non-limiting example of a monomer having variable light and/or heavy chain regions includes cDNA clones of anti-NAMPT antibody genes containing monomers formed of a scFv2 V_{H} chain and a scFv1 V_{L} chain, the chains connected by a linker.

A further non-limiting example of a monomer having variable light and/or heavy chain regions includes cDNA clones of anti-NAMPT antibody genes containing monomers formed of a scFv1 V_{H} chain and a scFv2 V_{L} chain, the chains connected by a linker.

In another aspect of the disclosure, each monomer includes 6 Complementarity Determining Regions (CDR) per scFv antibody, identified as CDR1, CDR2, CDR3, CDR4, CDR5, and CDR6. These CDRs confer the ability to recognize and bind to a unique antigen, in this case NAMPT.

In another aspect of the disclosure as illustrated in Tables 1 and 2 below, an exemplary scFv1 CDR1 has a nucleotide sequence identified as SEQ ID NO 9 and an exemplary amino acid sequence identified as SEQ ID NO 10. An exemplary scFv2 CDR1 has a nucleotide sequence identical to scFv1 CDR1 and identified as SEQ ID NO 9, and an exemplary scFv2 CDR1 has an amino acid sequence identical to scFv1 CDR1 and identified as SEQ ID NO 10. An exemplary scFv1 CDR 2 has a nucleotide sequence identified as SEQ ID NO 11 and an exemplary amino acid sequence identified as SEQ ID NO 12. An exemplary scFv2 CDR 2 has a nucleotide sequence identified as SEQ ID NO 13 and an exemplary amino acid sequence identified as SEQ ID NO 14. An exemplary scFv1 CDR3 has a nucleotide sequence identified as SEQ ID NO 15 and an exemplary amino acid sequence identified as SEQ ID NO 16. An exemplary scFv2 CDR3 has a nucleotide sequence identified as SEQ ID NO 17 and an exemplary amino acid sequence identified as SEQ ID NO 18. An exemplary scFv1 CDR4 has a nucleotide sequence identified as SEQ ID NO 19 and an exemplary amino acid sequence identified as SEQ ID NO 20. An exemplary scFv2 CDR4 has a nucleotide sequence that is identical to scFv1 CDR4 and identified as SEQ ID NO 19. The amino acid sequence of scFv2 CDR 4 is identical to scFv1 CDR4 and identified as SEQ ID NO 20. An exemplary scFv1 CDR5 has a nucleotide sequence identified as SEQ ID NO 21 and an exemplary amino acid sequence identified as SEQ ID NO 22. An exemplary scFv2 CDR5 has a nucleotide sequence identified as SEQ ID NO 23 and an exemplary amino acid sequence identified as SEQ ID NO 24. An exemplary scFv1 CDR6 has a nucleotide sequence identified as SEQ ID NO 25 and an exemplary amino acid sequence identified as SEQ ID NO 26. An exemplary scFv2 CDR6 has a nucleotide sequence identified as SEQ ID NO 27 and an exemplary amino acid sequence identified as SEQ ID NO 28.

TABLE 1
Amino acid sequence of
CDR for scFv1 and scFv2.

| | scFv1 | scFv2 |
|---|---|---|
| CDR1 | SYAMS | SYAMS |
| CDR2* | VINVPGNRTSYADSVK | NISGRGAKTTYADSVK |
| | GRFT** | GRFT** |
| CDR3* | KTRRFDY | TYLVFDY |
| CDR4 | RASQSISSYLN | RASQSISSYLN |
| CDR5* | GASVLQS | RASSLQSG |
| CDR6* | RVRSPAT | ISATPTT |

*CDR amino acid sequence differs between scFv1 and scFv2
**Unique sequence by BLAST

TABLE 2
Nucleotide sequence of
CDR for scFv1 and scFv2.

| | scFv1 | scFv2 |
|---|---|---|
| CDR1 | AGCTATGCCATGAGC | AGCTATGCCATGAGC |
| CDR2* | GTGATTAATGTTCCT | AATATTTCTGGGCGG |
| | GGTAATCGTACATCG | GGTGCTAAGACAACG |
| | TACGCAGACTCCGTG | TACGCAGACTCCGTG |
| | AAGGGCCGGTTCACC** | AAGGGCCGGTTCACC** |
| CDR3* | AAGACGCGTCGGTTT | ACGTATCTTGTGTTT |
| | GACTAC** | GACTAC** |
| CDR4 | CGGGCAAGTCAGAGC | CGGGCAAGTCAGAGC |
| | ATTAGCAGCTATTTA | ATTAGCAGCTATTTA |
| | AAT | AAT |
| CDR5* | GGTGCATCCGTTTTG | CGTGCATCCTCTTTG |
| | CAAAGT** | CAAAGTGGG** |
| CDR6* | CGTGTTCGTTCTCCT | ATTTCGGCTACGCCT |
| | GCTACG** | ACTACG** |

*CDR nucleotide sequences differs between scFv1 and scFv2
**Unique sequence by BLAST

The disclosure further provides that, within monomers, the scFv1 Complimentary Determining Regions and scFv2 Complimentary Determining Regions may be substituted for each other, where a CDR1 would be substituted for a CDR1, a CDR2 would be substituted for a CDR2, and so on. As previously discussed, scFv1 CDR1 and scFv2 CDR1, each having a nucleotide sequence identified as SEQ ID NO 9 and an amino acid sequence identified as SEQ ID 10, are identical; scFv1 CDR4 and scFv2 CDR4, each having a nucleotide sequence identified as SEQ ID NO 19 and an amino acid sequence identified as SEQ ID 20, are identical. Thus, scFv1 CDR 2 nucleotide SEQ ID NO 11 and scFv2 CDR2 nucleotide SEQ ID NO 13 may be substituted for each other. ScFv1 CDR 2 amino acid SEQ ID NO 12 and scFv2 CDR2 amino acid SEQ ID NO 14 may also be substituted for each other. ScFv1 CDR3 nucleotide SEQ ID NO 15 and scFv2 CDR3 nucleotide SEQ ID NO 17 may be substituted for each other. ScFv1 CDR3 amino acid SEQ ID NO 16 and scFv2 CDR3 amino acid SEQ ID NO 18 may also be substituted for each other. ScFv1 CDR5 nucleotide SEQ ID NO 21 and scFv2 CDR5 nucleotide SEQ ID NO 23 may be substituted for each other. ScFv1 CDR5 amino acid SEQ ID NO 22 and scFv2 CDR5 amino acid SEQ ID NO 24 may also be substituted for each other. ScFv1 CDR6 nucleotide SEQ ID NO 25 and scFv2 CDR6 nucleotide SEQ ID NO 27 may be substituted for each other. ScFv1 CDR6 amino acid SEQ ID NO 26 and scFv2 CDR6 amino acid SEQ ID NO 28 may also be substituted for each other. These CDR regions may be substituted in any monomer of the present disclosure, where the monomer may be part of a dimer, trimer, tetramer, and the like, as described herein. Therefore, one of skill in the art can appreciate the multitude of combinations possible for purposes of the present disclosure.

The disclosure further provides that, in any of the potential monomer, dimer, trimer and tetramer combinations previously described, within any monomer, one or more of the Complimentary Determining Regions within the V_{H} and V_{L} regions can be substituted between scFv1 and scFv2 as described above. This is in addition to the possibility of substituting the V_{H} and V_{L} regions within each monomer, where the monomer may be part of a dimer, trimer or tetramer, as described herein.

The disclosure further provides, as a non-limiting example, that in a scFv1 monomer, Complimentary Determining Region CDR2 may have nucleotide SEQ ID NO 13 and amino acid sequence SEQ ID NO 14 from the scFv2 fragment. Similarly, in another non-limiting example, a scFv2 monomer, CDR2 may have nucleotide SEQ ID NO 11 and amino acid SEQ ID NO 12 from scFv1. In a further non-limiting example, a scFv1 monomer, CDR3 may have nucleotide SEQ ID NO 17 and amino acid sequence SEQ ID NO 18 from scFv2. In yet a further non-limiting example, a scFv2 monomer, CDR3 may have nucleotide SEQ ID NO 15 and amino acid SEQ ID NO 16 from scFv1. In a further non-limiting example, a scFv1 monomer, CDR5 may have nucleotide SEQ ID NO 23 and amino acid sequence SEQ ID NO 24 from scFv2. Similarly, in another non-limiting example a scFv2 monomer, CDR5 may have nucleotide SEQ ID NO 21 and amino acid SEQ ID NO 22 from scFv1. In a scFv1 monomer, CDR6 may have nucleotide SEQ ID NO 27 and amino acid sequence SEQ ID NO 28 from scFv2. Similarly, in a scFv2 monomer, CDR6 may have nucleotide SEQ ID NO 25 and amino acid SEQ ID NO 26 from scFv1. Within any monomer, one or more of the foregoing substitutions can be made. For example, in an scFv1 monomer, CDR2 may have nucleotide SEQ ID NO 13 and amino acid sequence SEQ ID NO 14 from the scFv2 fragment, and CDR5 may have nucleotide SEQ ID NO 23 and amino acid sequence SEQ ID NO 24 from the scFv2 fragment.

In one embodiment, the present disclosure provides a dimer formed of an scFv1 connected with an scFv2 by a linker, as a non-limiting example, in the scFv1 monomer, CDR2 may have a nucleotides sequence SEQ ID NO 13 and amino acid sequence SEQ ID NO 14 from the scFv2 fragment, and CDR5 may have nucleotide SEQ ID NO 23 and amino acid sequence SEQ ID NO 24 from the scFv2 fragment; and in the scFv2 monomer, CDR2 may have nucleotide SEQ ID NO 11 and amino acid sequence SEQ ID NO 12 from the scFv1 fragment, and CDR5 may have nucleotide SEQ ID NO 21 and amino acid sequence SEQ ID NO 22 from the scFv1 fragment.

In another embodiment, the present disclosure provides a trimer formed of two scFv1 monomers and one scFv2 monomers, the monomers connected by linkers. As a non-limiting example, in one scFv1 monomer, CDR2 may have nucleotide SEQ ID NO 13 and amino acid sequence SEQ ID NO 14 from the scFv2 fragment, and CDR5 may have nucleotide SEQ ID NO 23 and amino acid sequence SEQ ID NO 24 from the scFv2 fragment; and in the scFv2 monomer, CDR2 may have nucleotide SEQ ID NO 11 and amino acid sequence SEQ ID NO 12 from the scFv1 fragment, and CDR5 may have nucleotide SEQ ID NO 21 and amino acid sequence SEQ ID NO 22 from the scFv1 fragment.

In a further embodiment, the present disclosure provides a tetramer formed of two scFv1 monomers and two scFv2 monomers, the monomers connected by linkers, as a non-limiting example, in each of the scFv1 monomers, CDR2 may have nucleotide SEQ ID NO 13 and amino acid sequence SEQ ID NO 14 from the scFv2 fragment, and CDR5 may have nucleotide SEQ ID NO 23 and amino acid sequence SEQ ID NO 24 from the scFv2 fragment; and in each of the scFv2 monomers, CDR2 may have nucleotide SEQ ID NO 11 and amino acid sequence SEQ ID NO 12 from the scFv1 fragment, and CDR5 may have nucleotide SEQ ID NO 21 and amino acid sequence SEQ ID NO 22 from the scFv1 fragment.

Another aspect of the present disclosure concerns use of peptide linker sequences of various lengths to connect individual scFv from the V_{H} chain of immunoglobulin to individual scFv1 and sFv2 from the V_{L} chain of immunoglobulin to form monomers, and to connect various scFv1 and scFv2 monomers to form dimers, tribodies and tetrabodies. Such peptide linkers may be used in any of the embodiments described above that utilize a linker.

Another aspect concerns the use of peptide linker sequences having different amino acid compositions to connect individual scFv1 and or individual scFv1 to form dimers, trimers and tetramers. Such peptide linkers may be used in any of the embodiments described above that utilize a linker.

In a further aspect, the present disclosure provides antibodies expressed by the cDNA clones of anti-NAMPT genes. Such antibodies are the proteins expressed by the cDNA clones described herein, where such cDNA clones may provide monomers, dimers, trimmers, or tetramers, having variable V_{H} and V_{L} regions and/or variable CDR regions as described herein.

In yet another aspect, the present disclosure provides the use of such antibodies, as described above, to inhibit NAMPT. In one embodiment, the use of such antibody clones to inhibit NAMPT is limited to specific neutrophil populations, where such neutrophil populations may be associated with a cancer, disease, disorder, or other illness.

In some embodiments of the present disclosure, the use of anti-NAMPT genes are preferably used to drive the anti-NAMPT antibody genes specifically expressed in neutrophils.

A further aspect of the present disclosure provides the therapeutic use of such antibodies to inhibit NAMPT-mediated cell proliferation, induce cell death and decrease NAD synthesis. In such an embodiment, the use of the antibodies, as described herein may be used to inhibit NAMPT in specific neutrophil, fibroblast, or macrophage populations, where such populations may be associated with a cancer, disease, disorder, or other illness.

The present disclosure additionally provides for a method of using the antibodies of the present disclosure for anti-NAMPT-targeted therapy for diseases whose pathogenesis involves an augmented expression of the NAMPT gene to drive the NAD inflammatory pathway. In one such an embodiment, the anti-NAMPT therapy is targeted to specific fibroblast, neutrophil, or macrophage populations associated with a particular disease state.

The method of the present disclosure for using the antibodies of the present disclosure for anti-NAMPT-targeted therapy, are preferably directed towards, but not limited to use in acute lung injury (ALI), acute respiratory distress syndrome (ARDS), juvenile idiopathic arthritis (JIA), rheumatoid arthritis (RA), cancer, and Inflammatory Bowel Diseases (IBD) including ulcerative colitis and Crohn's disease as well as other diseases in which an upregulation of NAMPT gene is a phenotype.

The present disclosure also provides for the therapeutic use of knockdown of neutrophil-specific NAMPT gene expression to treat LPS-, mechanical ventilation-, or LPS+mechanical ventilation-induced lung injury.

The present disclosure also provides for the therapeutic use of macrophage-specific knockdown of NAMPT gene expression to treat LPS-, mechanical ventilation-, or LPS+mechanical ventilation-induced lung injury.

The present disclosure additionally provides for the therapeutic use of fibroblast-specific knockdown of NAMPT gene expression to treat RA, JIA, osteoarthritis and osteoporosis.

The present disclosure also concerns NAMPT involvement in regulating expression of SP-B in the lung epithelial cells in vitro and in vivo. LPS or TNF-α stimulation increased NAMPT expression in both of H441 cells and A549 cells. Down regulation of NAMPT increased the expression of SP-B, as well as rescued the TNF-α induced inhibition of SP-B, while overexpression of NAMPT inhibited SP-B expression. NAMPT-induced inhibition of SP-B expression was mainly due to intracellular NAMPT nonenzymatic function via the JNK pathway, and partly due to enzymatic function. Mice harboring club cell specific deletion of NAMPT exhibited attenuated ALI and increased SP-B expression than wild type mice. Moreover, in epithelial cells, specific knockdown of NAMPT via recombinant virus may provide therapeutic potential to attenuate inflammation associated with ALI.

The present disclosure also concerns NAMPT mediated TNF-α induced inhibition of SP-B in H441 cells and A549 cells. NAMPT involves a lot of inflammatory processes. TNF-α augments NAMPT expression in A549 cells. The present disclosure confirmed the same role of NAMPT in H441 cells.

The present disclosure also concerns NAMPT regulation of SP-B expression via its NAMPT activity.

The present disclosure also concerns JNK pathway involvement in the NAMPT-inhibited SP-B in H441 cells.

The present disclosure also concerns epithelial cell specific knockdown of NAMPT and its effect on acute lung injury.

The present disclosure also concerns the generation of a heterozygous NAMPT L+/− mouse line with targeted deletion of a single NAMPT allele in epithelial cells, in order to examine the NAMPT function in vivo.

The present disclosure also concerns the therapeutic effect of the Ad-SPC-NAMPT antibody gene upon ALI.

The present disclosure also concerns a constructed adenovirus that expresses a NAMPT scFV antibody (Ad-SPC-NAMPT-scFv) driven by the lung epithelium specific human SPC promoter utilizing the Adeno-X™ Adenoviral System 3.

Various objects, features and advantages of this disclosure will become apparent from the following detailed description, which, taken in conjunction with the accompanying drawings, which depict, by way of illustration and example, certain embodiments of these anti-NAMPT antibody genes.

The drawings constitute a part of this specification, include exemplary embodiments of the anti-NAMPT antibody genes, and illustrate various objects and features thereof.

### BRIEF DESCRIPTION OF THE DRAWINGS

FIG. 1A is a flow chart showing amplification and ligation of a cDNA encoding scFv fragment to EcoRI, Kpn1 digested pCAGGS in accordance with the disclosure;

FIG. 1B is an illustration of the predicted structure of the scFv protein of FIG. 1A;

FIG. 1C is a graphic representation of the results of assay of cell proliferation of 3T3-L1 cells transfected with scFV1 and scFv2 or pCAGGS control. After 72 h, cell proliferation was assayed by CellTiter 96® AQueous One Solution Cell Proliferation Assay (Promega, cat #G3582). Values are means±SD, N=12, * p≤0.001;

FIG. 1D is a graphic representation of the results of assay of NAD synthesis by 3T3-L1 cells transfected with scFv1 or pCAGGS control. After 72 h NAD synthesis was measured by Amplite™ Fluorimetric NAD/NADH Ratio Assay Kit (AAT Bioquest®, Inc. prod. number 15263). Values are means±SD, N=4, p≤0.005. Ab1, antibody 1; Ab2, antibody 2;

FIG. 2 shows micrographs of stained lung sections from mice subjected to either PBS or LPS+MV, taken at 40× magnification;

FIG. 3A is a graphic representation of BAL total protein in mice subjected to either PBS or LPS+MPV N≥6/per group. *, p≤0.01 vs PBS groups; #, p<0.05 vs WT group w/LPS+MV;

FIG. 3B is a graphic representation of lung MPO in mice subjected to either PBS or LPS+MPV N≥6/per group *, p<0.01 vs PBS groups; #, p<0.05 vs WT group w/LPS+MV;

FIG. 3C is a graphic representation of BAL total cells in mice subjected to either PBS or LPS+MPV N≥6/per group *, p<0.01 vs PBS groups; #, p<0.05 vs WT group w/LPS+MV;

FIG. 3D is a graphic representation of total neutrophils in mice subjected to either PBS or LPS+MPV N≥6/per group. *, p<0.01 vs PBS groups; #, p<0.05 vs WT group w/LPS+MV;

FIG. 4A-C depict C57BL/6 mouse bone marrow neutrophil purity, chemotaxis and phagocytosis; In FIG. 4A, the purity of isolated neutrophils is >97.5% Gr-1 and CD 11b positive; FIG. 4B is representative images of neutrophils phagocytosis; FIG. 4C depicts neutrophil chemotaxis. Results are presented as percentage of RFU based on WT as 100%. N=4, *, p<0.05;

FIG. 5 A depicts representative gel images of CLP-induced KC, iL 1β, and IL-67 expressions in neutrophils specific NAMPT knockdown mouse lung tissues;

FIG. 5 B is a graphic representation of quantitative analysis of relative lung cytokine mRNA expressions in CLP-induced KC, iL 1β, and IL-67 expressions in neutrophils specific NAMPT knockdown mouse lung tissues with the controls set as 1. N≥5/per group. *, p<0.05 vs NAMPT^{N+/+}; #, p<0.05 vs NAMPT^{N+/-};

FIG. 6 is a depiction of molecular structures of the new NAMPT inhibitors synthesized from MC4-PPEA;

FIG. 7 A depicts stronger inhibitive effects on TNFα-induced translocation of NF_{K}B into nuclei in A549 cells than in FK 866;

FIG. 7B is a graphic representation of stronger inhibitive effects on thrombin-induced decrease in transendothelian resistance in HMVEC-L cells than FK866;

FIG. 8A depicts representative gel images of stronger MC4-PPEA inhibitory effect on lung IL-6 and IL-1β expression compared with FK866 in CLP-sepsis-induced lung injury;

FIG. 8B depicts quantitative analysis of relative lung cytokine mRNA expressions for lung IL-6 and IL-1β expression compared with FK866 in CLP-sepsis-induced lung injury with the controls set as 1. N≥5/per group. *, p<0.05 vs DMSO group; #, p<0.05 vs FK866 group. DM, DMSO; MC4, MC4-PPEA; FK, FK866;

FIG. 9A illustrates a neutrophil targeted MRP8-NAMPT shRNA adenoviral expression vector;

FIG. 9B illustrates a neutrophil targeted MRP8-NAMPT CDNA adenoviral expression vector;

FIG. 9C illustrates a four-copy NAMPT shRNA expression v3ector;

FIG. 9D illustrates a western blot analysis of NAMPT protein expression in mouse 3T3-L1 fibroblasts transfected with NAMPT shRNA expression vectors; C=Control, no DNA; Sc1=1 copy scrambled shRNA, 1C=1 NAMPT shRNA; Sc4=4 copies scrambled shRNA; 4C−1=4 copies of identical NAMPT shRNA; 4C-D=4 copies of 4 distinct NAMPT shRNA.

FIG. 10 is a graphic representation of BAL protein concentration of NAMPT shRNA treated mice compared with NAMPT cDNA treated mice. Mice were injected via their tail veins with each purified recombinant adenovirus (3×10⁹ pfu) for 24 h followed by LPS+MV as done in FIG. 3 before the quantification of BAL protein concentrations. N=3, *, p<0.01;

FIG. 11A is a graphic representation of inflammation and bone erosion in collagen induced arthritis in NAMPT⁺/⁻ mice with NAMPT⁺/⁺ and NAMPT⁺/⁻ mice (n=8 NAMPT⁺/⁺ and 10 NAMPT⁺/⁻ mice, * P value<0.05;

FIG. 11B is a graphic representation of serum levels of NAMPT and anti-collagen antibody of FIG. 12A (scale×100,000) (n=3 NAMPT⁺/⁺ CIA, n=7 NAMPT⁺/⁻ CIA;

FIG. 11C illustrate MicroCT image analysis for left foot and talus of mice of FIG. 12A (the arrow represents location of talus in ankle joint);

FIG. 11D is a graphic representation of mean/density of bone volume of talus presented as mean+/−standard deviation of mice of FIG. 12A (n=3 NAMPT⁺/⁺′ n=3 NAMPT⁺/⁺ CIA, n=7 NAMPT⁺/⁻ CIA);

FIG. 12A illustrates RNA sequence analysis of whole ankle joints from DBA1J CIA and control mice using a heat map of 171 genes upregulated in NAMPT^{+/+} CIA, but not in NAMPT^{+/-} CIA. (N=3);

FIG. 12B illustrates cellular assembly and organization, tissue development and connective tissue disorders network;

FIG. 12C illustrates gene expression, cell-mediated immune response, and cellular development network (gray shading represents genes within the network that are upregulated);

FIGS. 13A-C illustrate inhibition of LPS-induced TRAP cells by NAMPT knockdown in RAW 264.7 cells. Cells in 13A were nucleofected with 116siRNA and scrambled control, stimulated with LPS (10 ng/ml) for 24 h and stained for TRAP. 13B illustrates the frequency of TRAP positive cells after 24 h LPS treatment expressed as mean±SD (N=3). 13C is a Western blot of NAMPT knockdown by 116siRNA in RAW 264.7 cells.

FIG. 14A. illustrates the molecular structure of a mouse NAMPT transgene construct overexpressing NAMPT^{OE};

FIGS. 15A, B, and C illustrate generation and characterization of NAMPT^{N-/-} mice (FIG. 14A provides a roadmap for generating NAMPT^{N-/-} mice. Electroporation and balstoccyst injection of ES cells were performed in The Transgenic Animal Core, U. of Missouri. FIG. 15B illustrates genotyping mouse Neu and organs from NAMPT^{F/F}×Mrp8−Cre− mice and shows the Neu specific NAMPT gene deletion. FIG. 15C illustrates phenotyping of NAMPT protein expression in NAMPT^{F/F}×Mrp8-Cre mice shows the lowest level in Neu. GAPDH was used as a loading control. Neu, neutrophils; C+, Cre+; C−, Cre−);

FIG. 16A illustrates results of assay of NAMPT promoter SNPs and haplotypes in luciferase reporter gene constructs;

FIG. 16B illustrates selected haplotypes resulting from cloning of the 1974 bp NAMPT promoter upstream of pGL3-Basic and mutagenization of the SNPs to generate haplotypes;

FIG. 17 illustrates identification of SNPs in Human NAMPT Gene Promoter;

FIG. 18 illustrates an overview of the omega-based B1H System (FIG. 18A illustrates the oligos for the −1535 28mer binding site were annealed to form NotI and EcoRI overhangs and cloned into digested pH3U3 and sequenced verified. FIG. 18B illustrates template switching cDNA synthesis was initiated using the CDC XbaI reverse primer and Clontech's SMART™ technology. Three primers were used to capture each ORF. The cDNA library was digested with Kpn1 and XbaI and cloned into the pB1H2 digested vector. FIG. 18C illustrates recruitment of TF-omega fusions to the promoter driving the HIS3 and URA3 selection reporters;

FIG. 19 illustrates LPS and TNF-α stimulated NAMPT protein expression in H441 cells. H441 cells were treated without or with different doses of LPS (A) or TNF-α (C) for 24 hours. An equal amount of total cell lysate protein (10 μg) from each sample was separated by 15% SDS-PAGE and immunodetected by western blotting using anti-human NAMPT or GAPDH antibodies. Protein bands were quantified (B, D) by densitometry using image J, and NAMPT levels were normalized to GAPDH levels. NAMPT levels in control cells were arbitrarily set at 1. Values are means±SD of 3 independent experiments. *P<0.05 vs. control cells;

FIG. 20 illustrates NAMPT blunted LPS or TNF-α inhibition of SP-B expression. (A) H441 cells were transfected with either scramble RNA (Sc RNA) or NAMPT siRNA for 48 hours before treatment without or with either LPS (10 μg/ml) or TNF-α (25 ng/ml) for 24 hours. Cell lysate of NAMPT (10 μg), SP-B (30 μg) and GAPDH (10 μg) protein were detected by western blot (A). Protein bands were quantified by densitometry using image J, and NAMPT levels (B) and SP-B levels (C) were normalized to GAPDH levels. NAMPT levels and SP-B levels in Sc RNA cells without LPS treatment were arbitrarily set at 1. Values are means±SD of 3 independent experiments. *p<0.05 vs. Sc RNA cells without LPS or TNF-α treatment; #p<0.05 vs Sc RNA cells with either LPS or TNF-α treatment;

FIG. 21 illustrates NAMPT inhibited SP-B expression via both its nonenzymatic and enzymatic activity. (A) H441 cells were transfected with either pCAGGS, pCAGGS-hNAMPT or pCAGGS-H247E. Cell lysate of NAMPT (10 μg), SP-B (30 μg) and GAPDH (10 μg) proteins were detected by western blot. Protein bands were quantified by densitometry using image J, and NAMPT levels (B) and SP-B levels (C) were normalized to GAPDH levels. Values are means±SD of 3 independent experiments. *p<0.05 vs. pCAGGS cells; #p<0.05 vs pCAGGS-hNAMPT cells. (D) H441 cells were pretreated with different doses of FK866 for 6 h before treatment without or with TNF-α (25 ng/ml) for 24 hours. Cell lysate of NAMPT (10 μg), SP-B (30 μg) and GAPDH (10 μg) proteins were detected by western blot. Protein bands were quantified by densitometry using image J, and NAMPT levels (E) and SP-B levels (F) were normalized to GAPDH levels. Values are means±SD of 3 independent experiments. *p<0.05 vs cells without FK866 or rhTNF-α treatment. #p<0.05 vs cells pretreated with FK866 but without rhTNF-α treatment;

FIG. 22 illustrates that the JNK pathway is involved in the NAMPT inhibition of SP-B expression in H441 cells. (A) H441 cells were pretreated without or with either p38 inhibitor or JNK inhibitor for 6 hours before transfection with pCAGGS-hNAMPT or pCAGGS-H247E. Cell lysate of SP-B (30 μg) and GAPDH (30 μg) proteins were detected by western blot. Protein bands were quantified by densitometry using image J, and SP-B (B) were normalized to GAPDH. Values are means±SD of 3 independent experiments. *p<0.05 vs control cells without inhibitor treatment;

FIG. 23 illustrates the attenuation of lung injury in NAMPT^{L+/-} mice on LPS induced ALI. (A) PCR genotyping mouse lung and organs from NAMPT^{F/F}×Lyz-Cre-mice showed the lung specific NAMPT gene deletion. (B) Western blot of NAMPT and SP-B expression in an equal volume of bronchoalveolar lavage (BAL) supernatant from NAMPT^{L+/+} mice and NAMPT^{L+/-} mice treated with LPS (2 mg/kg) for 24 hours. NAMPT^{L+/-} mice presented with decreased total BAL cells count (C), total BAL polymorphonuclear neutrophils (PMNs) (D), BAL protein concentration (E), and BAL TNF-α level (F) in LPS (2 mg/kg; 24 hours)-induced lung injury model compared to NAMPT^{L+/+} mice treated with the same conditions. (G) Representative hematoxylin and eosin images and bar graph quantifications demonstrating that LPS induced significant increases in lung injury compared with spontaneous breathing control mice. NAMPT^{L+/-} mice had reduced amounts of inflammation, edema, and alveolar damage seen in the LPS induced lung injury model compared to NAMPT^{L+/+} mice. (Scale bar, 100 μm. Original magnification ×200). (H) Lung injury scores of NAMPT^{L+/-} mice were significantly lower than those of NAMPT^{L+/+} mice in LPS induced ALI models. Lung injury score was defined as Percent Alveoli Damage×Cell Density. (I) PCR and its quantification of IL-6, TNF-α expression in lung tissue from NAMPT^{L+/+} mice and NAMPT^{L+/-} mice treated with either PBS or LPS. N≥5/per group. *, p<0.05 vs PBS groups; #, p<0.05 vs WT group w/LPS;

FIG. 24 illustrates that administration of SPC-NAMPT antibody gene in adenovirus (Ad-SPC-NAMPT AB) reduced LPS-induced murine lung injury. (A) Fluorescence of H441 and A549 cells infected with Ad-SPC-NAMPT antibody. (B) Western blot of NAMPT and SP-B expression in H441cells infected with Ad-SPC-NAMPT antibody. (C) Wild type mice were injected with either Ad-control insert or SPC-NAMPT antibody gene intratracheally. The mice were challenged with either PBS or LPS (2 mg/kg, 24 hours) intratracheally 3 days later. Bronchoalveolar lavage (BAL) and lung tissue samples were collected. Administration of Ad-SPC-NAMPT antibody attenuated increases in BAL total cells (C), BAL neutrophils (D), BAL protein concentration (E), inflammatory cell infiltration (F) and lung injury scores (G) in LPS-challenged mice. Scale bar, 100 μm. Original magnification ×200. N≥5/per group. *P<0.05 compared to PBS control group. #P<0.05 compared to LPS and SC shRNA group; and

FIG. 25 illustrates the construction of NAMPT scFv antibody tissue specific expression vectors. A. NAMPT scFv cDNA under the control of the strong, constitutive synthetic CAGGS promoter. B. NAMPT scFv cDNA under the control of the neutrophil specific human MRP8 promoter. C. NAMPT scFv cDNA under the control of the mouse SPC3.7 lung-specific promoter.

### DETAILED DESCRIPTION

Production and manipulation of the cDNA clones for the single-chain variable fragment scFv1 and scFv2 anti-NAMPT antibodies described herein are within the skill in the art and can be carried out according to recombinant techniques described, among other places, in Maniatis, et al., 1989, Molecular Cloning, A Laboratory Manual, Cold Spring Harbor Laboratory Press, cold Spring Harbor, N.Y.; Ausubel, et al., 1989, Current Protocols in Molecular Biology, Greene Publishing Associates & Wiley Interscience, NY; Sambrook, et al. 1989, Molecular Cloning: A Laboratory Manual, 2d ed., Cold Spring Harbor Laboratory Press, Cold Spring Harbor, N.Y.; Innis et al. (Eds.), 1995, PCR Strategies, Academic Press, Inc., San Diego; and Erlich (ed), 1992, PCR Technology, Oxford University Press, New York, all of which are incorporated herein by reference.

In preparing the cDNA clones, any expression vector appropriate for the intended recipient may be employed. Examples of appropriate expression vectors include but are not limited to: bacteria, yeasts, virus derivatives such as plasmids, bacteriophages, animal viruses, retroviruses, baculoviruses, and combinations thereof.

As used herein, the term “linker” refers to any peptide of from about 10 to about 25 amino acids or any other peptide known to work with these types of cDNA sequences. A non-limiting example of such linkers include the 18 mer linker, HIV1 p24 linker, 15-mer (G4S)₃ linker.

The present disclosure provides two unique anti-NAMPT antibody genes scFv1 and scFv2 that were identified by screening a HuScL-2™ Phage Display Naïve Human scFv Library against purified recombinant NAMPT protein. The scFv1 and scFv2 clones contain complementary determining regions having unique nucleotide and amino acid sequences.

The scFv1 and scFv2 clones vary in the heavy chain V_{H} and light chain V_{L} chain regions and in the six Complementarity Determining Regions CDR1-CDR6, that is to say SEQ ID NOs 10-28. Preferably, the fragments of the present disclosure are from about 96% to about 97% identical.

In a preferred embodiment, the cDNA clones of the present disclosure have at least 96% sequence homology to either the nucleotide sequences SEQ ID No. 1 or SEQ ID No. 3, where sequences homology values of at least 97%, at least 98%, at least 99%, and 100% sequence homology are envisioned. In a preferred embodiment, the cDNA clones of the present disclosure have at least 96% sequence homology to either the amino acid e sequences SEQ ID No. 2 or SEQ ID No. 4, where sequences homology values of at least 97%, at least 98%, at least 99%, and 100% sequence homology are envisioned. Further, it is understood that codon optimization may be performed and that the sequence homology comparison be performed on the encoded amino acid sequences.

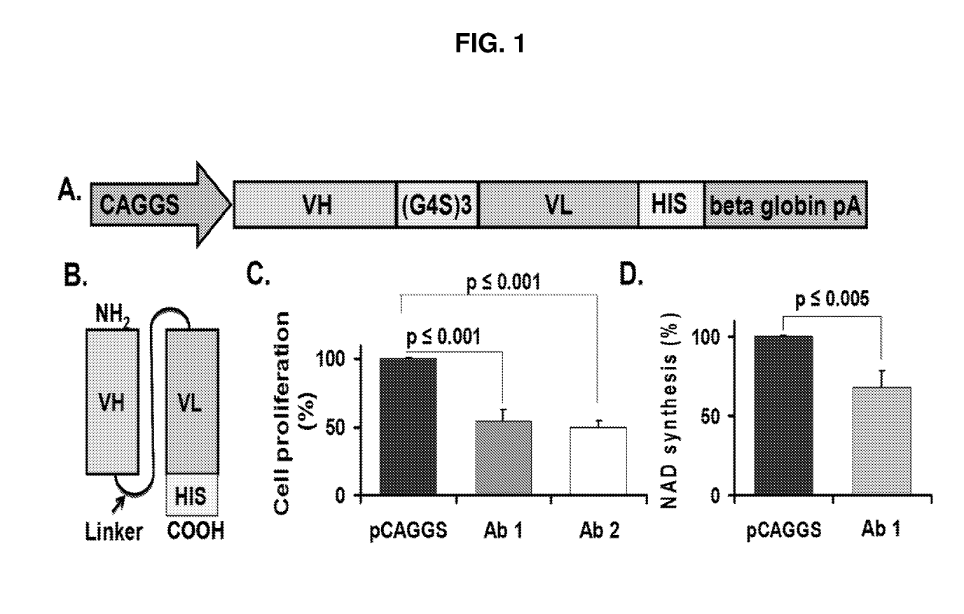

In cell culture experiments, the scFv1 and scFv2 clones were found to inhibit cell proliferation and induce cell death. In one embodiment of the present disclosure, as best shown in FIG. 1A, the cDNA for each single-chain variable fragment (scFv) antibody was cloned, including a histidine tag on the 3′ end, into the pCAGGS expression vector. The V_{H} and V_{L} fragments for each scFv are separated by a (G4S)3 linker and encode proteins of 247 and 249 amino acids, respectively for NAMPT-scFv1 (antibody 1 or Ab1) and scFv2 (antibody 2 or Ab2) (FIG. 1B). To test the anti-NAMPT ability of each construct, MTS assays were performed in transfected 3T3-L1 (ATCC CL-173™) and RAW 264.7 (ATCC® TIB-71™) cells. Preliminary results showed that tested anti-mouse antibody clones Ab1 and Ab2 inhibited proliferation of mouse 3T3 (FIG. 1C), macrophage cell lines and human a549 cell (data not shown) by inhibiting NAMPT since NAMPT has an antiapoptotic function. The tested Ab1 also inhibited cell NAD synthesis (FIG. 1D) via inhibiting NAMPT since NAMPT is a key enzyme in cell NAD synthesis of a most important mammalian salvage pathway.

As shown in FIG. 1C, the scFv1 and scFv2 clones (Ab1 and ab2) have been used to target selected populations of fibroblasts. Each of the scFv1 and scFv2 clones decreased proliferation by about 50% as compared to the pCAGGS control population. Thus, in one embodiment of the present disclosure the cDNA clones of the present disclosure and the antibodies that are formed from the proteins created by the amino acid sequences preferably reduce fibroblast populations by at least 50%, where values such as at least 60% reduction, at least 70% reduction, at least 80% reduction, at least 90% reduction, and at least 95% reduction are envisioned. FIG. 1C values (Avg±SD): Ab1 54.3%+0.8.8; Ab2 49.8%+4.6. FIG. 1D values (Avg±SD): Ab1 68.3%+10.4.

The technology described herein is relevant for any physiological process or disease involving the NAD inflammatory pathway, including cancer, infections, autoimmune diseases and other genetic disorders, acute respiratory distress syndrome, arthritis, cancer, coronary artery disease, and diabetes. It may be employed to target selected populations of neutrophils associated with any injury, illness, or disease state associated with neutrophil proliferation.

In therapeutic uses, such cDNA clones can be expressed into a cell using a lentiviral or adenoviral system and then injected into the body of an animal or a human to deliver the purified antibody protein to the body for treatment of an inflammatory disease. In another aspect, the cDNA clones may also be injected directly into the body. In another aspect, the cDNA clones may be introduced to the body orally or sublingually. Targeted cells may include neutrophils, macrophages, fibroblasts, endothelials, or other cells where a cell specific promoter may drive the cell specific expression of anti-NAMPT gene.

In another aspect, NAMPT is used to mediate TNF-α induced inhibition of SP-B in epithelial cells. Knockdown of NAMPT increases SP-B expression, and NAMPT mediates TNF-α induced inhibition of SP-B in H441 cells and A549 cells, both of which are epithelial cells.

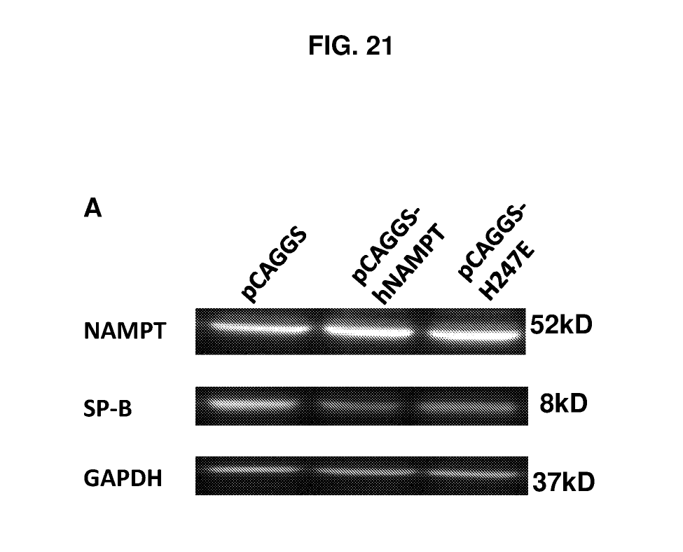

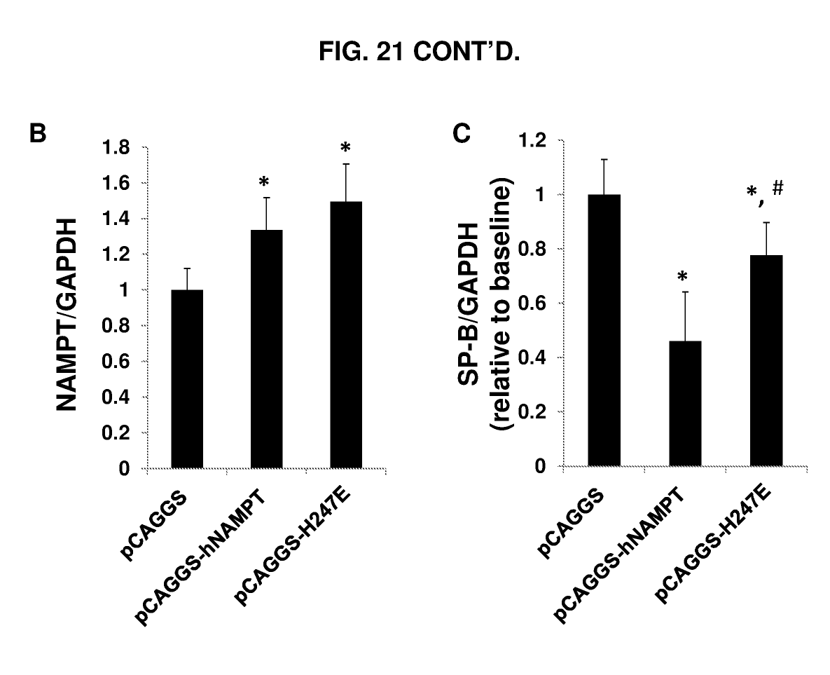

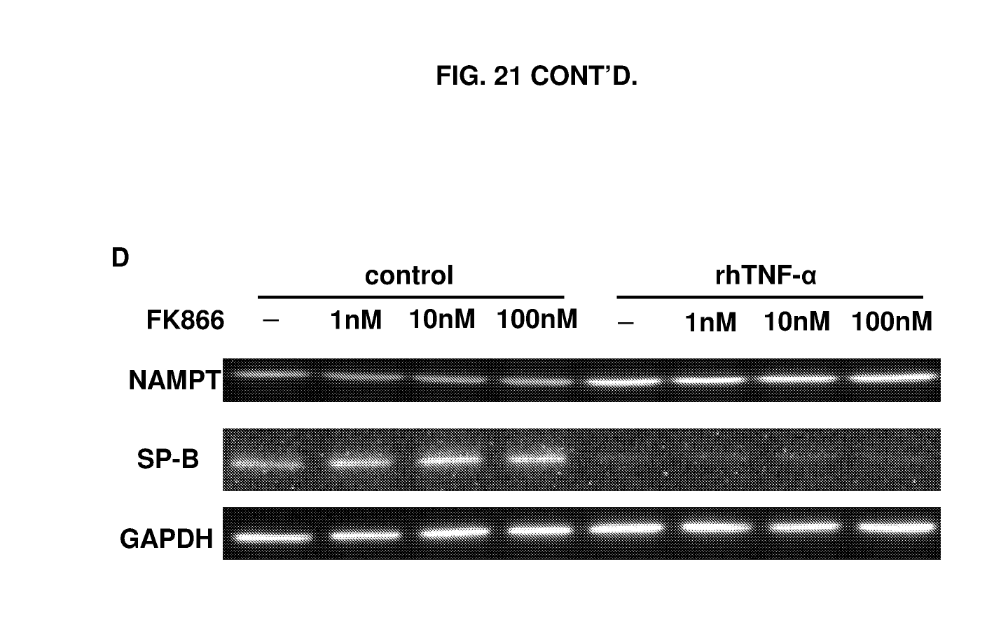

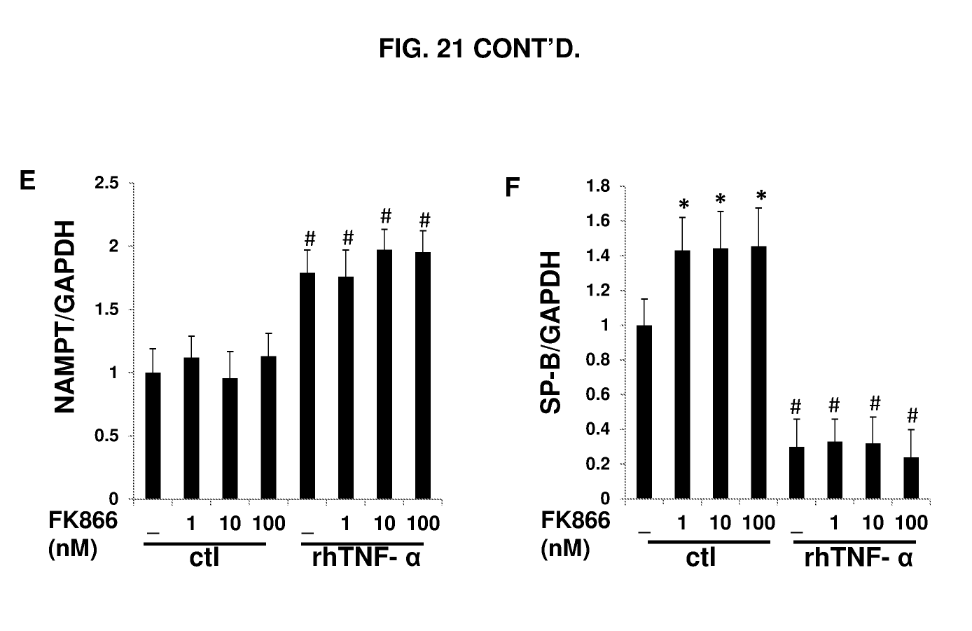

In another aspect, NAMPT is used to inhibit SP-B expression mainly via its nonenzymatic activity and partly via its enzymatic activity. FIG. 21A shows that overexpression of mutant NAMPT similarly and significantly inhibited the SP-B expression protein levels as with wild-type NAMPT.

In another aspect of the present disclosure, the JNK pathway is used to regulate NAMPT-inhibition of SP-B in epithelial cells.

In another aspect of the present disclosure, cell specific knockdown of NAMPT in epithelial cells is used to attenuate acute lung injury in mice.

In another aspect, the Ad-SPC-NAMPT antibody gene is used to achieve a therapeutic effect on ALI.

In another aspect, a lung epithelial cell specific NAMPT knockdown is used as a new therapeutic modality in ALI.

The present disclosure provides for an anti-NAMPT cDNA clone having a sequence at least 96% homologous to nucleotide sequence SEQ ID No. 1, wherein said clone has a complementary determining region 2 selected from the group consisting of SEQ ID No. 11 and SEQ ID No. 13.

The present disclosure provides for an anti-NAMPT cDNA clone encoding a sequence at least 96% homologous to amino acid sequence SEQ ID No. 2, wherein said clone has a complementary determining region 2 selected from the group consisting of SEQ ID No. 12 and SEQ ID No. 14.

The present disclosure provides for an anti-NAMPT cDNA clone having a sequence at least 96% homologous to nucleotide sequence SEQ ID No. 3, wherein said clone has a complementary determining region 2 selected from the group consisting of SEQ ID No. 11 and SEQ ID No. 13.

The present disclosure provides for an anti-NAMPT cDNA clone encoding a sequence at least 96% homologous to amino acid sequence SEQ ID No. 4, wherein said clone has a complementary determining region 2 selected from the group consisting of SEQ ID No. 12 and SEQ ID No. 14.

The present disclosure provides for an anti-NAMPT cDNA clone having a sequence at least 96% homologous to nucleotide sequence SEQ ID No. 1, wherein said clone has a complementary determining region 3 selected from the group consisting of SEQ ID No. 15 and SEQ ID No. 17.

The present disclosure provides for an anti-NAMPT cDNA clone encoding a sequence at least 96% homologous to amino acid sequence SEQ ID No. 2, wherein said clone has a complementary determining region 3 selected from the group consisting of SEQ ID No. 16 and SEQ ID No. 18.

The present disclosure provides for an anti-NAMPT cDNA clone having a sequence at least 96% homologous to nucleotide sequence SEQ ID No. 3, wherein said clone has a complementary determining region 3 selected from the group consisting of SEQ ID No. 15 and SEQ ID No. 17.

The present disclosure provides for an anti-NAMPT cDNA clone encoding a sequence at least 96% homologous to amino acid sequence SEQ ID No. 4, wherein said clone has a complementary determining region 3 selected from the group consisting of SEQ ID No. 16 and SEQ ID No. 18.

The present disclosure provides for an anti-NAMPT cDNA clone having a sequence at least 96% homologous to nucleotide sequence SEQ ID No. 1, wherein said clone has a complementary determining region 5 selected from the group consisting of SEQ ID No. 21 and SEQ ID No. 23.

The present disclosure provides for an anti-NAMPT cDNA clone encoding a sequence at least 96% homologous to amino acid sequence SEQ ID No. 2, wherein said clone has a complementary determining region 5 selected from the group consisting of SEQ ID No. 22 and SEQ ID No. 24.

The present disclosure provides for an anti-NAMPT cDNA clone having a sequence at least 96% homologous to nucleotide sequence SEQ ID No. 3, wherein said clone has a complementary determining region 5 selected from the group consisting of SEQ ID No. 21 and SEQ ID No. 23.

The present disclosure provides for an anti-NAMPT cDNA clone encoding a sequence at least 96% homologous to amino acid sequence SEQ ID No. 4, wherein said clone has a complementary determining region 5 selected from the group consisting of SEQ ID No. 22 and SEQ ID No. 24.

The present disclosure provides for an anti-NAMPT cDNA clone having a sequence at least 96% homologous to nucleotide sequence SEQ ID No. 1, wherein said clone has a complementary determining region 6 selected from the group consisting of SEQ ID No. 25 and SEQ ID No. 27.

The present disclosure provides for an anti-NAMPT cDNA clone encoding a sequence at least 96% homologous to amino acid sequence SEQ ID No. 2, wherein said clone has a complementary determining region 6 selected from the group consisting of SEQ ID No. 26 and SEQ ID No. 28.

The present disclosure provides for an anti-NAMPT cDNA clone having a sequence at least 96% homologous to nucleotide sequence SEQ ID No. 3, wherein said clone has a complementary determining region 6 selected from the group consisting of SEQ ID No. 25 and SEQ ID No. 27.

The present disclosure provides for an anti-NAMPT cDNA clone encoding a sequence at least 96% homologous to amino acid sequence SEQ ID No. 4, wherein said clone has a complementary determining region 6 selected from the group consisting of SEQ ID No. 26 and SEQ ID No. 28.

The present disclosure provides for a dimer comprising any two anti-NAMPT cDNA clones individually and respectively selected from the clones described herein.

The present disclosure provides for a trimer comprising any three of the anti-NAMPT cDNA clones individually and respectively selected from the clones described herein.

The present disclosure provides for a tetramer comprising any four of the anti-NAMPT cDNA clones individually and respectively selected from the clones described herein.

The present disclosure provides for a cDNA clone as described herein, wherein the heavy chain of SEQ ID No. 1 is replaced by the heavy chain of SEQ ID No. 3.

The present disclosure provides for a cDNA clone as described herein, wherein the light chain of SEQ ID No. 1 is replaced by the light chain of SEQ ID No. 3.

The present disclosure provides for a cDNA clone of anti-NAMPT antibody genes having the nucleotide sequence of SEQ ID No. 1.

The present disclosure provides for a cDNA clone of anti-NAMPT antibody genes encoding the amino acid sequence of SEQ ID No. 2

The present disclosure provides for a cDNA clone of anti-NAMPT antibody genes having the nucleotide sequence of SEQ ID No. 3.

The present disclosure provides for a cDNA clone of anti-NAMPT antibody genes encoding the amino acid sequence of SEQ ID No. 4

The present disclosure provides for a cDNA clone of anti-NAMPT antibody genes having the nucleotide sequence of SEQ ID No. 5.

The present disclosure provides for a cDNA clone of anti-NAMPT antibody genes encoding the amino acid sequence of SEQ ID No. 6.

The present disclosure provides for a method of inhibiting the NAMPT pathway, wherein said method comprises administration of any one of the cDNA clones described herein.

The present disclosure provides for a method of targeting neutrophil populations, wherein said method comprises administration of any one of the cDNA clones described herein.

The present disclosure provides for a method for treating LPS-induced lung injury, wherein said method comprises administration of any one of the cDNA clones described herein

### EXAMPLES

Examples I-III are directed to neutrophil chemotaxis, activation and apoptosis between NAMPT+/+ and NAMPT N−/− mice in basal and challenged conditions.

### Example I

### Substantiation of the Role of a Neutrophil Specific NAMPT Expression in the Pathogenesis of ALI by Adoptive Transfer of Neutrophils from NAMPT Overexpression (NAMPT^{OE}) Mice or NAMPT Heterozygous Knockdown (NAMPT^{+/-}) Mice into Wild Type (NAMPT^{+/+}) Mice

Activation and transmigration of neutrophils is a hallmark event in the progression of ALI and ARDS. Since it has an antiapoptotic role, NAMPT functions to prolong neutrophil presence at the site of inflammation, and hence results in hyperinflammatory tissue damage because of the neutrophil's capacity for the production of toxic mediators. Knockdown of NAMPT in neutrophils would serve to enhance neutrophil apoptosis, shorten the life of neutrophils, which could ameliorate long lasting neutrophil-related inflammatory change.

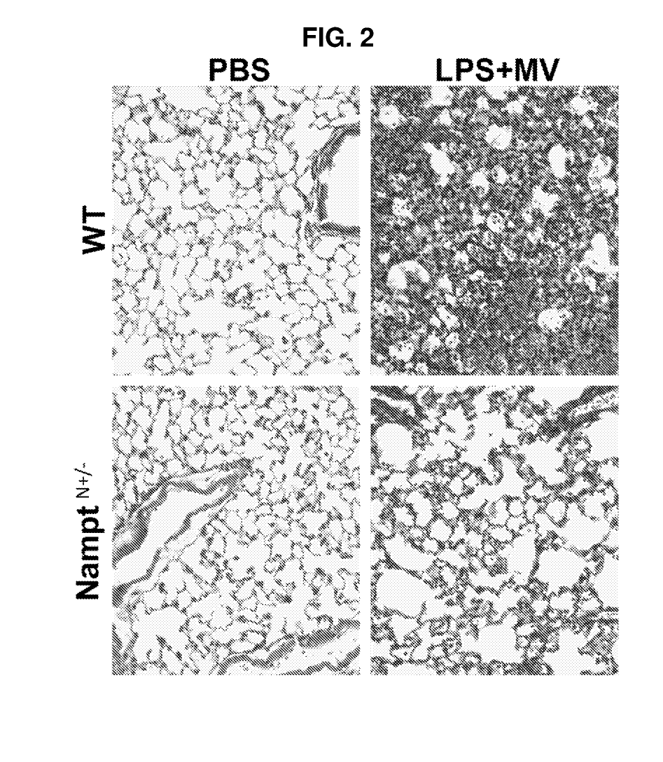

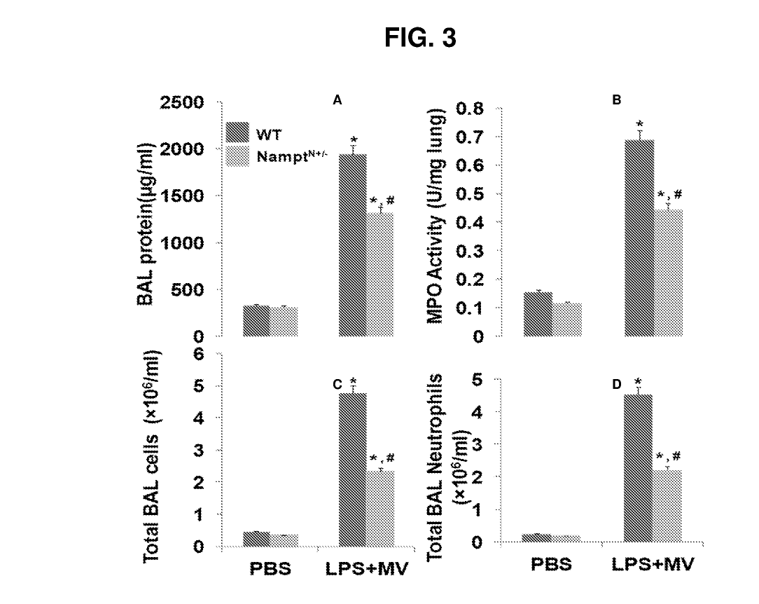

Neutrophils from bone marrows of either NAMPT^{OE} or NAMPT^{+/-} or NAMPT^{+/+} mice are isolated using The Neutrophil Isolation Kit (cat. No. 130-097-658, Miltenyi Biotec Inc., San Diego, Calif.). Isolated neutrophils with the purity >95% as determined by flow cytometry using CD11b and Gr-1 staining and the viability >98% as determined by PI exclusion are used. Three different donor NAMPT^{OE} or NAMPT^{+/-} or NAMPT^{+/+} neutrophils (5×106 cells each) plus a control (0.2 ml PBS) are adoptively transferred into four recipient groups of NAMPT^{+/+} mice by an injection in the retro-orbital sinus according to the protocol of Boari et al. 48 h later, those four recipient groups of mice are subject to LPS+MV as carried out in our preliminary experiments (FIGS. 2-3) according to our previously published methods with a minor modification. Briefly, mice are anesthetized with ketarnine/acepromazine, intubated intratracheally, and administered with LPS (1.5 μg/g body weight, E. coli 0111:B4, Cat No. L4391, Sigma) for 20 h before ventilator placement (room air; tidal volume, 30 ml/kg; 65 breaths/min; 0 cm H₂O positive end-expiratory pressure) with a SomnoSuite™ Small Animal Anesthesia System (Kent Scientific Corp., Torrington, Conn., USA) for 4 h. After the challenge with LPS+MV, mouse BAL and lung tissues are harvested to determine BAL total protein, total and differential cell counts, TNFα and MPO levels and lung histology, MPO, TNFα and IL1β levels. These parameters inform us which neutrophil transferred recipient mice show an aggravation or attenuation or no change in the pathogenesis of ALI compared to the control. Age- and gender-matched C57BL/6 mice between 8 and 12 weeks old, n=6/per group, are used in this study. This sample size (n=6 mice/per group) has a power of 0.985 to detect at least 0.15±0.05 (mean±SD, ng/dl) difference in the concentration of BAL protein with alpha value set as 0.05 based on 4 group ANOVA power calculation using Sigmaplot analysis software (ver 13, Systat Software, Inc., San Jose, Calif.).

Conclusion: An adoptive transfer of neutrophils from NAMPT overexpression (NAMPT^{OE}) mice or NAMPT heterozygous knockdown (NAMPT^{+/-}) mice into wild type (NAMPT^{+/+}) mice aggravates or attenuates the pathogenesis of ALI when challenged with LPS+mechanical ventilation (MV), further substantiating the role of a neutrophil specific NAMPT expression in the pathogenesis of ALI.

### Example II

### Determination of Functional Differences Between NAMPT^{+/+} and NAMPT^{+/-} Neutrophils Isolated from Mice in Basal and LPS+Ventilation Challenged Conditions

Neutrophils are the first line response of the innate immune system to injury, releasing cytotoxic mediators and reactive oxygen species (ROS). However, if neutrophils persist, they can result in host tissue damage. Therefore, the factors that control the recruitment, function, lifespan, and removal of these cells are important for host defense and resolution of inflammation.

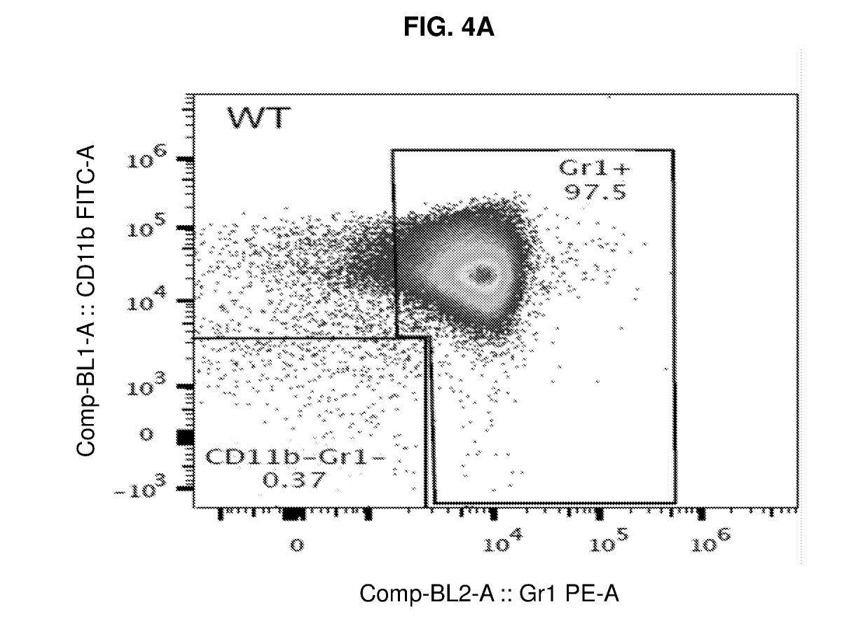

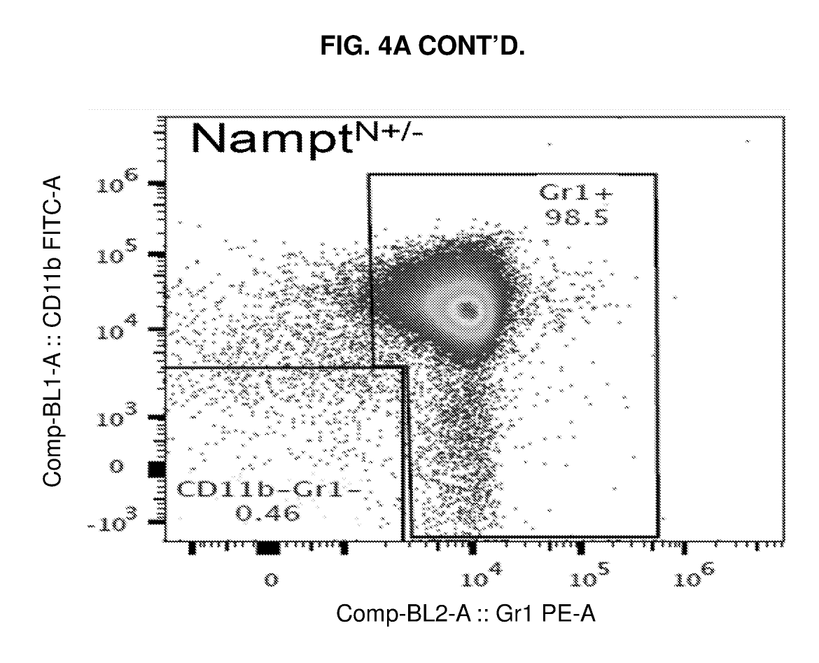

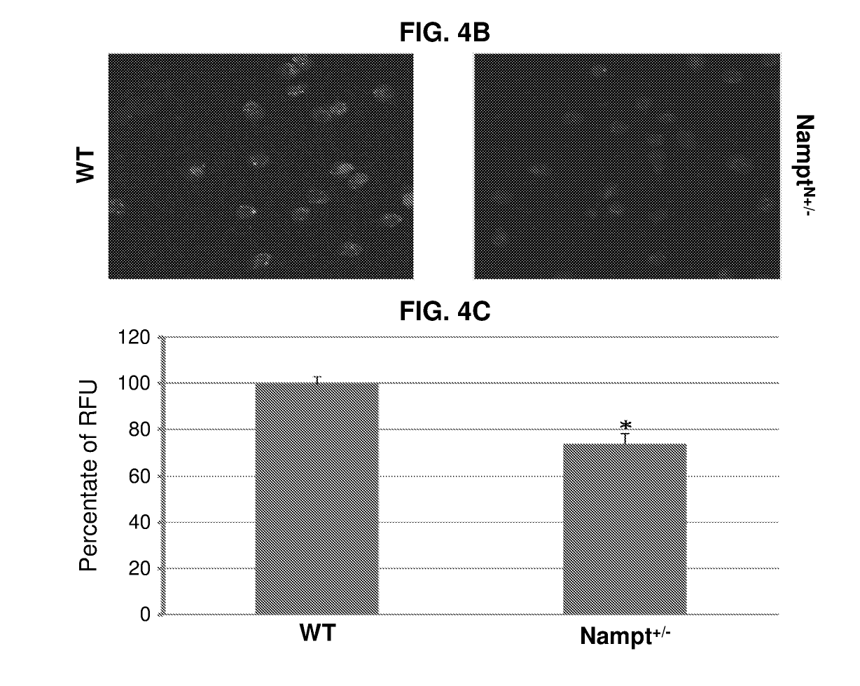

Neutrophils isolated from bone marrows of either NAMPT^{N+/-} or NAMPT^{+/+} mice are examined as described in Experiment I. Preliminary data have indicated that the purity of isolated neutrophils using the above-referenced kit reaches more than 97% Gr-1 and CD11b positive (FIG. 4A) as analyzed on Attune® Acoustic Focusing Cytometer (Thermo Fisher Sci) using the FlowJo software (found on the web at “flowjo.com/”).

### Chemotaxis:

Chemotaxis of those neutrophils isolated from bone marrows of either NAMPT^{N+/-} or NAMPT^{+/+} mice in basal or LPS+MV challenged conditions towards known chemoattractants (e.g. fMLP) is carried out using the CHEMICON® QCM™ Chemotaxis 3 μm 24-well Migration Assay in a Migration Chamber, based on the Boyden chamber principle (Cat. No. ECM505, EMD Milliport). Migrating cells are lysed and detected by the CyQUANT® GR dye (Molecular Probes).

Results: A chemotactic migration of NAMPT^{N+/-} neutrophils towards fMLP in baseline is significantly decreased compared to wild type counterparts though it is assayed in a limited number of mice (FIG. 4B)

### Activation:

Neutrophil activation status of those neutrophils isolated from bone marrows of either NAMPT^{N+/-} or NAMPT^{+/+} mice in basal or LPS+MV challenged conditions are assessed by measurement of neutrophil shape change and by CD62-L/CD11b expression levels using RT-PCR. Superoxide anion production is assessed by dihydrorhodamine fluorescence. Neutrophil TNFα and IL1-(3 level is measured by ELISA.

### Apoptosis:

Apoptosis of those neutrophils isolated from bone marrows of either NAMPT^{N+/-} or NAMPT^{+/+} mice in basal or LPS+ventilation challenged conditions are assessed by flow cytometric analysis for Annexin-V/propidium iodide staining and by analysis of cleaved caspase-3 by western blot and ELISA.

### Phagocytosis:

Phagocytic index of those neutrophils isolated from bone marrows of either NAMPT^{N+/-} or NAMPT^{+/+} mice in basal or LPS+MV challenged conditions are measured using a kit with pHrodo Green S. aureus BioParticle conjugates for phagocytosis (Cat. No. P35367, Thermo Fisher Scientific Inc.) to allow quantification of phagocytosis by flow cytometry and fluorescence microscopy. Our study using this assay did not find a significant difference in phagocytosis between wild type and NAMPT^{N+/-} neutrophils in baseline (FIG. 4C). Images were taken on a confocal microscope (Zeiss UV-LSM 510 Meta with a 63× objective, 1.4 N/A oil DIC).

### Statistical Analysis:

To evaluate differences in these variables between NAMPT^{+/-} and NAMPT^{+/+} neutrophils groups, parametric statistical analyses are performed using Sigmaplot analysis software (ver 13, Systat Software, Inc. San Jose, Calif. with an unpaired t-test (two groups) or ANOVA (multiple groups) followed by the Tueky-Kramer Multiple Comparisons posttest. Results are represented as mean±DS. Two-tailed P values<0.05 are considered significant.

The same principles of these statistical analyses are applied to all examples for data analysis.

Conclusion:

Neutrophils with the NAMPT gene knockdown may decrease their chemotactic migration and activation and apoptosis without compromising their ability in innate immunity compared to those wild type neutrophils

### Example III

### Use of RNA-Seq Technology to Profile Transcriptomes of Neutrophils from Both NAMPT^{+/+} Mice and NAMPT^{N+/-} Mice without or with LPS+MV Challenge

RNA-seq is a technology that uses next-generation sequencing to determine the identity and abundance of all RNA sequences in biological samples. RNA-seq profiling of neutrophil transcriptomes from both NAMPT^{+/+} mice and NAMPTN^{+/-} mice without or with LPS+MV challenge provides us with rich information on new molecular targets, new components in canonical pathways, and new pathways of gene expression, which may lead us to elucidate new and novel molecular mechanisms underlying the therapeutic effect of neutrophil specific knockdown of NAMPT on ALI/ARDS as well as neutrophil dependent and general pathogenesis of ALI.

Illumina's HiSeq1500 instrument is used to characterize the neutrophil transcriptomes from both NAMPT^{+/+} mice and NAMPTN^{+/-} mice without or with LPS+MV challenge, using our established protocol and data analysis pipeline. Each type of RNA samples includes at least three biological replicates. Total RNA (1 μg) of each sample is converted into a paired-end cDNA library using the Illumina's TruSeq Stranded Total Library Preparation kit (Cat. No. RS-122-2201) before sequencing following Illumina's protocol. Sequence quality analyses, alignments to reference genome, differential transcript calling are carried out according to our established pipeline except the transcript assembly which is done using String Tie, a new software using a genome-guided transcriptome assembly approach along with concepts from de novo genome assembly with the capacity to increase new transcript calls by 20 to 53%. Significantly differentially expressed transcripts (FDR, q<0.05) with >2 fold magnitude between NAMPT^{N+/-} and NAMPT^{+/+} neutrophils groups are subjected to pathway analysis of either QIAGEN'S Ingenuity Pathway Analysis (found on the web at “ingenuity.com/products/ipa”) or The Database for Annotation, Visualization and Integrated Discovery (DAVID, found on the web at “david.abcc.ncifcrf.gov”). Selected candidates are validated by RT-PCR before subjecting to further experimentation to gain insights into molecular mechanisms, therapeutic utility as new drug targets in ALI.

Conclusion:

A neutrophil specific knockdown of NAMPT gene can significantly attenuate acute lung injury, and neutrophils with NAMPT knockdown can decrease their chemotactic migration and activation and apoptosis without compromising their ability in innate immunity. RNA-seq provides information on new molecular targets, new components in canonical pathways, and new pathways of gene expression, which may lead to elucidation of new and novel molecular mechanisms underlying the therapeutic effect of a neutrophil specific knockdown of NAMPT gene on ALI/ARDS as well as neutrophil-dependent and general pathogenesis of ALI.

Examples 4-6 investigate the role of a neutrophil specific NAMPT knockdown in three different mouse models of ALI (LPS+MV, sepsis and pneumonia). LPS- or MV-induced animal models of ALI are the most widely used animal models of ALI. It is thought that the “two hits” such as LPS+MV synergize to lead to more detrimental effects on the lung, which is more closely mimicked to the pathogenesis of human ALI. Sepsis is one of the main risk factors for ARDS. Pulmonary infections are the main risk factor of ALI/ARDS in 46-51% patients. Sepsis and pneumonia are the most common causes of death among ALI/ARDS patients. Because of these, a sepsis induced ALI model, was selected in which cecal ligation and puncture (CLP) induced peritonitis is followed by sepsis and lung injury, and a pneumonia induced ALI model, in which local administration of bacteria into the lungs is achieved by an intratracheal catheter, to evaluate the broad therapeutic utility of neutrophil specific NAMPT knockdown on ALI/ARDS. The CLP procedure was used to induce sepsis because it is the most frequently used polymicrobial infection model and it closely resembles the progression and characteristics of human sepsis.

### Example IV

### Investigation of the Role of a Neutrophil Specific NAMPT Knockdown Across Three Overlapping Stages of ALI Pathogenesis: Exudative, Proliferative, and Fibrotic in LPS+MV Model of ALI

Age- and gender-matched C57BL/6 mice between 8 and 12 weeks old, n=6/per group, are used in this study. The sample size (n=6/per group) is based on the power calculation given at Experiment I. Mice are divided into four groups at each time point: NAMPT^{+/+} experiment (NAMPT^{+/+}-E), NAMPT^{+/+} control (NAMPT^{+/+}-C), NAMPT^{+/-} experiment (NAMPT^{+/-}-E), NAMPT^{+/-} control (NAMPT^{+/-}-C). Experimental groups are subjected to the LPS+MV treatment and control groups to the room air +PBS as described in Experiment I. After the experimentation, mouse BAL and Lung tissues from day 1 and 2 (exudative stage), day 3 and 5 (proliferative stage), day 7 and 10 (fibrotic stage) are harvested. Various assays in BAL (protein, total and differential cell counts, cytokines TNFα and IL1β) and lung tissues (wet/dry ratio, MPO, cytokines TNFα and IL1β, histological haematoxylin and eosin staining for visualizing alveolar structure, and Masson's Trichrome Staining for Collagen Fibers) are carried out as described by Patel et al and in our preliminary study (FIGS. 2-3, 5). These parameters inform us whether a neutrophil specific NAMPT knockdown has a long and lasting therapeutic effect on the recovery of injured lungs compared to the control after the LPS+MV insult.

### Example V

### Investigation of the Role of a Neutrophil Specific NAMPT Knockdown in CLP-Sepsis Induced Mouse Model of ALI

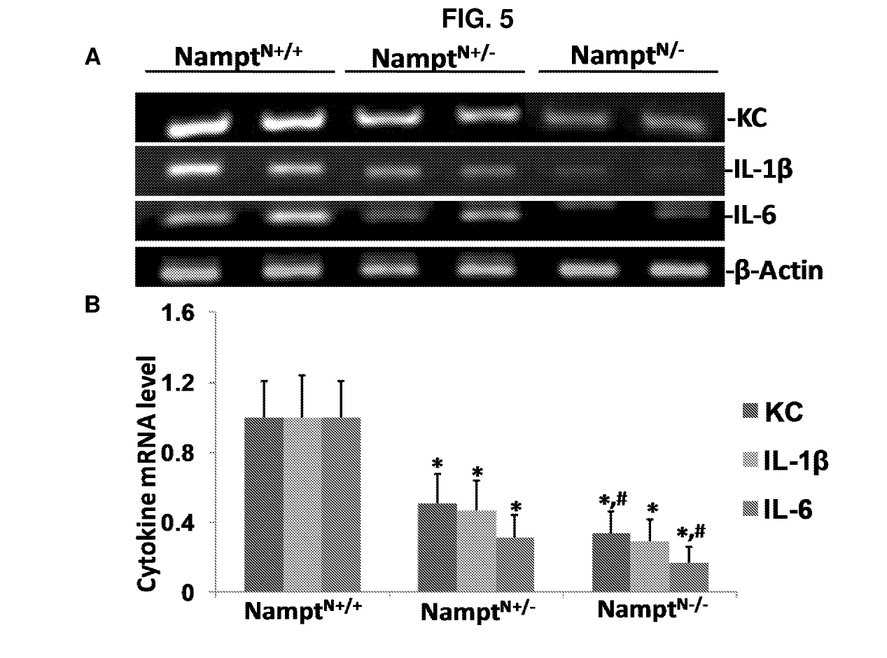

Age- and gender-matched C57BL/6 mice between 8 and 12 weeks old, n=6/per group, are used in this study. The sample size (n=8/per group) is based on the power calculation from our preliminary data (FIG. 5). Mice are divided into four groups at each time point: NAMPT^{+/+} experiment (NAMPT^{+/+}-E), NAMPT^{+/+} control (NAMPT^{+/+}-C), NAMPT^{+/-} experiment (NAMPT+/−-E), NAMPT+/−control (NAMPT+/−-C). Experimental groups will be subjected to the CLP procedure by Rittirsch et al. Briefly, a midline laparotomy is performed on the anesthetized mice. The distal 50% of exposed cecum is ligated with 3-0 silk suture and punctured with 1 pass of an 18-gauge needle. The incision is closed with 3-0 suture. Control groups with sham operations are subjected to midline laparotomy and manipulation of cecum without ligation and puncture. Postoperatively, the animals are resuscitated with 1 ml subcutaneous injection of sterile 0.9% NaCl. After the experimentation, mouse BAL, serum and Lung tissues from 4 h, 8 h, 16 h, 24 h, 3d and 5d are harvested. Various assays in BAL (protein, total and differential cell counts, cytokines TNFα and IL1β), blood (cytokines TNFα and IL1β) and lung tissues (wet/dry ratio, MPO, cytokines TNFα and IL1β, histological haematoxylin and eosin staining for visualizing alveolar structure) are carried out as described by Patel et al. and in our preliminary study (FIGS. 2-3, 5). These parameters inform us whether a neutrophil specific NAMPT knockdown has a protective role in CLP-sepsis induced mouse model of ALI.

### Example VI

### Investigation of the Role of a Neutrophil Specific NAMPT Knockdown in Bacteria-Pneumonia Induced Mouse Model of ALI

Age- and gender-matched C57BL/6 mice between 8 and 12 weeks old, n=6/per group, are used in this study. The sample size (n=6/per group) is extrapolated on the power calculation from our preliminary data (FIG. 5). Mice are divided into four groups at each time point: NAMPT^{+/+} experiment (NAMPT^{+/+}-E), NAMPT^{+/+} control (NAMPT+/+-C), NAMPT^{+/-} experiment (NAMPT^{+/-}-E), NAMPT^{+/-} control (NAMPT^{+/-}-C). Experimental groups are subjected to the bacteria-pneumonia procedure of Quinton et al. Briefly, 1×106 CFU Escherichia coli (serotype 06:K2:H1; ATCC #19138; American Type Culture Collection) are administered into the left lung via an intratracheal catheter after mice are anaesthetized. Control groups are given equal volume of saline. 24 h later, mouse BAL and Lung tissues is harvested. Various assays in BAL (protein, total and differential cell counts, cytokines TNFα and IL1β), and lung tissues (wet/dry ratio, MPO, cytokines TNFα and IL1β, histological haematoxylin and eosin staining for visualizing alveolar structure) are carried out as described by Quinton et al. and in our preliminary study (FIGS. 2-3, 5). In addition, bacteria load is assessed from mouse blood or lung homogenates by the serially dilutions of them in sterile H₂O and grown overnight at 37° C. on 5% sheep blood agar plates. Viable bacteria are enumerated by colony counts and expressed as total CFU per lung or per milliliter of blood. These parameters inform us whether a neutrophil specific NAMPT knockdown has a protective role in bacteria-pneumonia induced mouse model of ALI.

### Results

We witness a beneficial protective effect of neutrophil specific NAMPT knockdown on all three stages of ALI. Neutrophil specific NAMPT knockdown also protects against lung injury in either sepsis- or pneumonia-induced mouse model of ALI.

EXAMPLES 7-9 evaluate the therapeutic efficacy of neutrophil NAMPT targeted small chemical inhibitors, antibodies and shRNAs in ALI/ARDS. Small chemical inhibitors have been demonstrated as potential drugs for inflammatory diseases. Here we improve the structure of the small chemical inhibitor to NAMPT, MC4-PPEA, to evaluate the therapeutic efficacy on LPS+MV induced mouse model of ALI by a neutrophil targeted delivery of MC4-PPEA or its derivatives. scFv (Single-Chain Fragment Variable) antibodies have been successfully applied as diagnostic reagents and therapeutic gene deliveries. We improve the scFv antibody to NAMPT to generate neutrophil specific expression of anti-NAMPT gene as a new therapy to ALI. siRNA or shRNA-based therapeutics have demonstrated the capability to silence therapeutically relevant genes in various in vivo models of cancer, infections, autoimmune diseases, and other genetic disorders including ALI. We improve the forms of NAMPT shRNA to develop powerful and neutrophil specific NAMPT knockdown as a viable therapeutic strategy to ALI.

### Example VII

### Evaluation of the Therapeutic Efficacy of Neutrophil NAMPT Targeted Small Chemical Inhibitors in ALI/ARDS

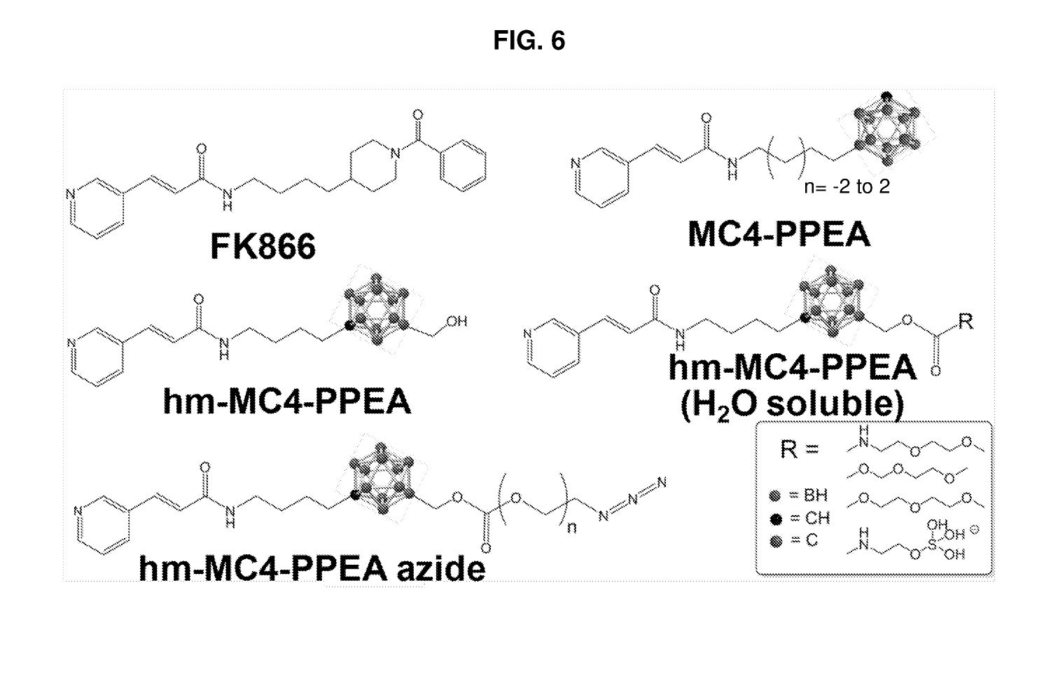

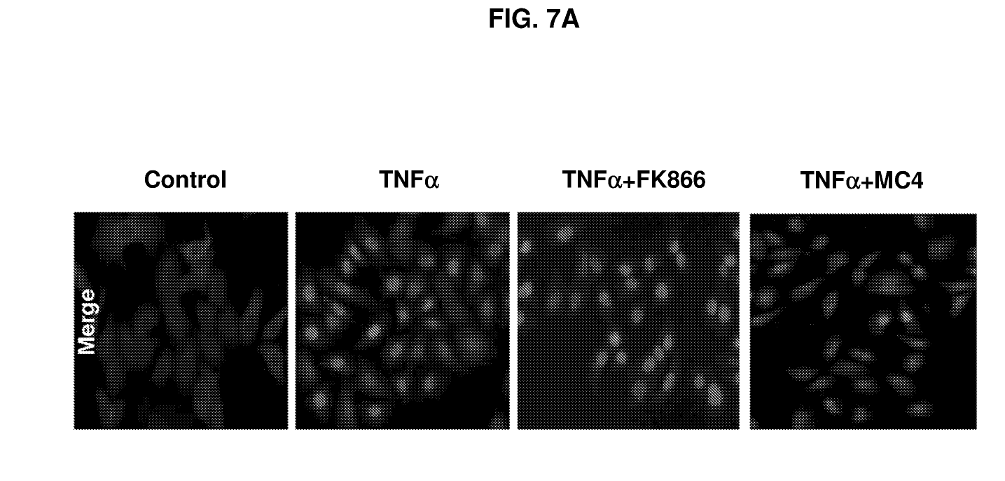

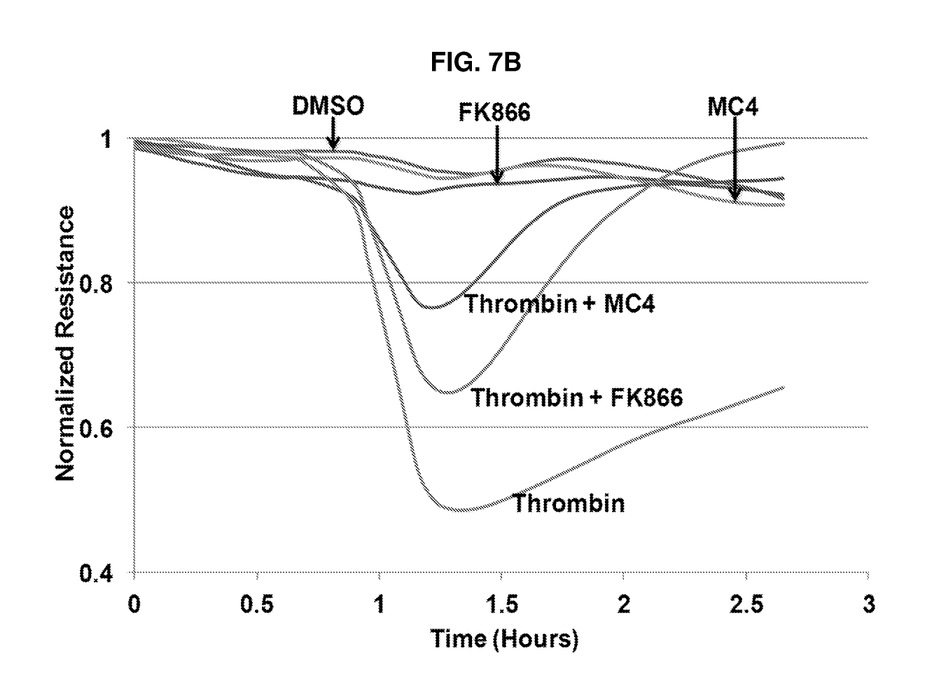

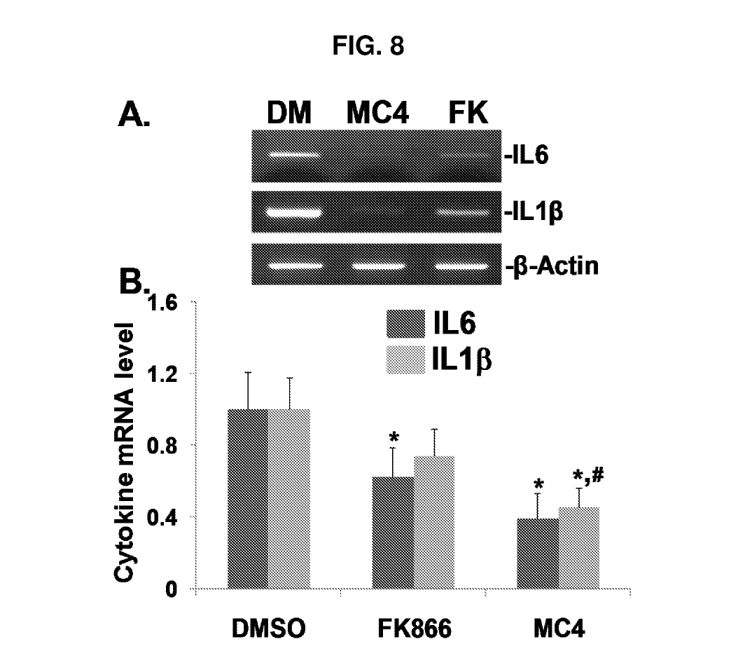

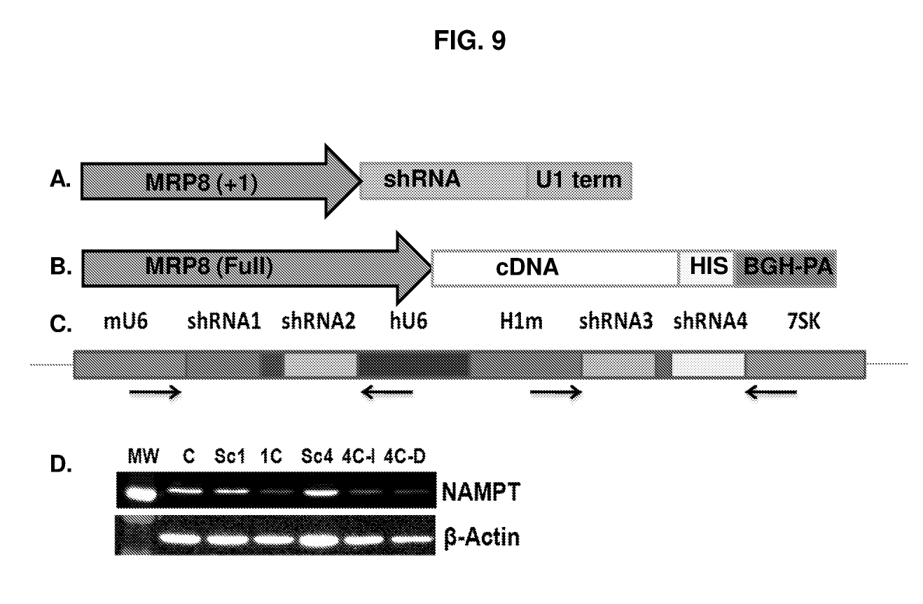

We previously modified FK866, an inhibitor of NAMPT, by replacing its benzoylpiperidine moiety with a meta-carborane to yield a more potent and less toxic NAMPT inhibitor, MC4-PPEA (FIG. 6), which displayed a 100-fold increase in NAMPT inhibition over FK866. Our preliminary data indicate that MC4-PPEA had a significant stronger inhibitory effect on NF_{K}B activation (FIG. 7A), endothelial cell permeability (FIG. 7B), CLP-induced mouse sepsis than its original counterpart, FK866 (FIG. 8). We first optimize the MC4-PPEA's structure in terms of the placement of the carborane moiety (FIG. 6), its water solubility and targeted delivery to neutrophils. Recent work in our labs has produced a new derivative bearing a hydroxymethyl moiety on the C-7 carbon atom of the cage (hm-MC4, FIG. 6). We have demonstrated that this group allows for the covalent attachment of polyethylene glycol, carbonates, carbamates, and esters, exhibiting varying hydrolytic stability. These new MC4-PPEA conjugates exhibit high potency against human cancer cells in vitro. This functionality will serve as the site for the covalent attachment of water solubilizing groups, such as a short methoxypolyethylene glycol chain, or a sulfate ester. Potent “next generation” NAMPT inhibitors bearing a sulfone moiety have recently been reported. We incorporate this moiety into the MC4 structure and investigate its effects on inhibitory activity and solubility. The activity of these new molecules is measured initially using NAMPT inhibitory assay in vitro.

We develop a neutrophil-specific NAMPT inhibitor by covalently linking the optimized hydroxymethyl MC4 compound (similar to hm-MC4 azide, FIG. 6) to a neutrophil targeting peptide such as GGPNLTGRW (GGP) as described by Karathanasis et al. To produce these new conjugates, we utilize click chemistry using a short azide terminated PEG linker and a peptide bearing an alkyne (FIG. 6). The binding avidity of the new conjugates for targeted tissue is measured in vitro using competition assays and blocking studies with fluorescent analogs. After the coupling, the neutrophil specific targeting is confirmed according to the method of Mazzucchelli et al. A control peptide whose sequence was generated from a scrambled GGP peptide sequence is also coupled to MC4-PPEA. It is used as both in vitro cell binding and in vivo neutrophil targeting control.

Twenty four wild type C56BL/6 mice with age, gender matched are arranged into 3 groups of 8 mice each. These three groups are intravenously injected with 0.5 mg MC4-PPEA-peptide, 0.5 mg control peptide, and saline according to the method of Newton-Northup et al. Two hours later, they are subjected to LPS+MV procedure as described in our preliminary study (FIGS. 2-3). At the 24 h point of LPS treatment, BAL and lung tissues of all mice are collected. Various assays in BAL (protein, total and differential cell counts, cytokines TNFα and IL1β) and lung tissues (wet/dry ratio, MPO, cytokines TNFα and IL1β, histological haematoxylin and eosin staining for visualizing alveolar structure, and Masson's Trichrome Staining for Collagen Fibers) are carried out as described by Patel et al and in our preliminary study (FIGS. 2-3). These parameters inform us whether neutrophil specific NAMPT knockdown by MC4-PPEA-GGP peptide delivery has a therapeutic effect on the injured lungs in LPS+MV mouse model of ALI by comparing all parameters in MC4-PPEA-GGP peptide group with those in control peptide group or saline group. Similar procedures are carried out to test newly synthesized derivatives of MC4-PPEA.

### Example VIII

### Evaluation of the Therapeutic Efficacy of Neutrophil NAMPT Targeted Antibodies in ALI/ARDS

We expressed and purified recombinant mouse NAMPT protein and obtained two human against mouse NAMPT antibody gene clones, termed NAMPT-scFv1 (Ab1) and -scFv2 (Ab2), by screening a HuScL-2™ Phage Display Naïve Human scFv Library (www.creative-biolabs.com/). We cloned the cDNAs for each scFv, including a histidine-tag on the 3′ end, into the pCAGGS expression vector. The VH and VL fragments for each scFv are separated by a (G4S)3 linker and encode proteins of 247 and 249 amino acids, respectively for NAMPT-scFv1 and -scFv2. To test the anti-NAMPT ability of each construct, we performed MTS assays in transfected 3T3-L1 (ATCC® CL-173™) and RAW 264.7 (ATCC® TIB-71™) cells. Our preliminary result shows that tested anti-mouse antibody clones, Ab1 and Ab2, can inhibit proliferation of mouse 3T3 (FIG. 1C) and macrophage cell lines (data not shown) by inhibiting NAMPT since NAMPT has an antiapoptotic function. The tested Ab1 also inhibited cell NAD synthesis (FIG. 1D) via inhibiting NAMPT since NAMPT is a key enzyme in the cell NAD synthesis of a most important mammalian salvage pathway. To increase the specificity of these constructs we generate a hetero-dimer and a hetero-tetramer using established procedures. The hetero-dimer [scFv1-scFv2], also called a di-body, is separated by a GGSSRSSSSGGGGSGGGG linker. The hetero-tetramer, also called a tetra-body, consists of two dimers [scFv1-scFv2]2 joined by the HIV1 P21 capsid protein linker, GATPQDLNTML. The P21 linker is an epitope for the murine monoclonal CB4-1 antibody. Similar to the mono-bodies (scFv1 and scFv2), the di-body and tetra-body possesses a His-Tag. Thus, we utilize the CB4-1 antibody and nickel-columns (His-Tag) to manipulate and purify our recombinant scFv antibodies. We enhance the specificity and tertiary structures of the di-body and tetra-body by modifying the type and length of the linker sequences. Their antisense clone of NAMPT-scFv1 and -scFv2 is used as controls. Since similar effects in in vitro assay were observed, only NAMPT-scFv1 and its antisense clone are used to prepare the constructs under the control of either the human MRP8 promoter or mouse MPO promoter for tissue specific expression in neutrophils or neutrophils and monocytes, respectively.

To test in vivo therapeutic effect, eighteen wild type C56BL/6 mice with age, gender matched are arranged into 2 groups of 8 mice each. These 2 groups are intravenously injected with NAMPT-scFv1 or its antisense control plasmid. 24 hours later, they are subjected to LPS+MV procedure as described in our preliminary study (FIGS. 2-3). At the 24 h point of LPS treatment, BAL and lung tissues of all mice are collected. Various assays in BAL (protein, total and differential cell counts, cytokines TNFα and IL1β) and lung tissues (wet/dry ratio, MPO, cytokines TNFα and IL1β, histological haematoxylin and eosin staining for visualizing alveolar structure, and Masson's Trichrome Staining for Collagen Fibers) are carried out as described by Patel et al. and in our preliminary study (FIGS. 2-3). These parameters inform us whether neutrophil specific NAMPT knockdown by NAMPT-scFv1 has a therapeutic effect on the injured lungs in LPS+MV mouse model of ALI by comparing all parameters in NAMPT-scFv1 group with those in antisense control antibody group. Similar procedures are carried out to test newly synthesized dibody and tetrabody of NAMPT antibody.

### Example IX

### Evaluation of the Therapeutic Efficacy of Neutrophil NAMPT Targeted shRNAs in ALI/ARDS

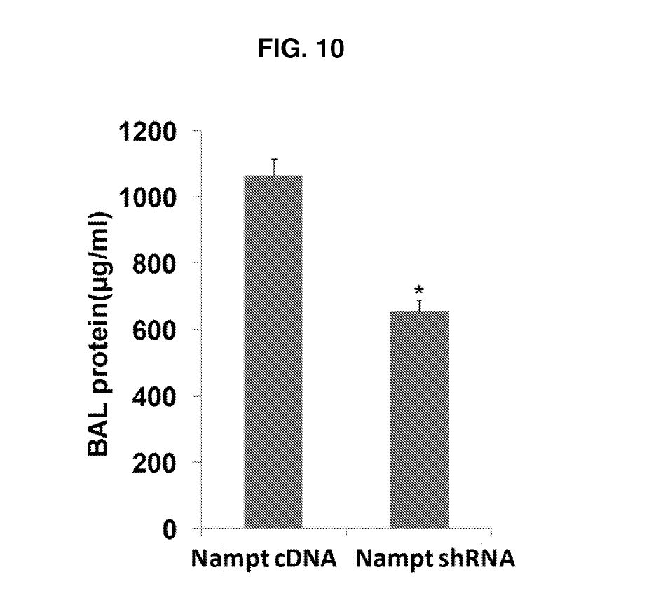

We have constructed tissue specific adenoviral expression vectors utilizing the Adeno-X™ Adenoviral System 3 (Cat #632264) with In-Fusion HD cloning technology and Stellar Competent E. coli cells (Clontech® Laboratories, Inc.). We have generated a NAMPT shRNA (NAMPT: 971: 5′ TGAAGACCTGAGACATCTGATA 3′) expression vector using truncated hMRP8 promoters according to the strategy of Giering et al., to optimize shRNA expression using tissue specific pol II promoters (FIG. 9A). A NAMPT scramble shRNA expression vector was similarly constructed (data not shown). We also constructed a histidine-tagged mouse NAMPT cDNA expression vector driven by the human MRP8 promoter (FIG. 9B). A NAMPT anti-sense cDNA expression vector was similarly constructed (data not shown). The recombinant constructs were terminated by bovine growth hormone poly A or U1 Box 3 signals for NAMPT shRNA and cDNA, respectively. Plasmid DNAs from positive clones of each construct were sequence-verified and digested with Pad prior to transfection of Adeno-X 293 cells for production of recombinant adenovirus. Following the overlap extension PCR procedure of Gou et al., we constructed expression vectors (pK4-shRNA) expressing either 4 identical or 4 different shRNA molecules, each under the control of a unique pol III promoter (FIG. 9C). These constructs efficiently knock-down NAMPT expression in vitro (FIG. 9D). The very first test in a limited scale informed us that our two constructs (MRP8-NAMPT shRNA and MRP8 NAMPT cDNA) seems to function as expected: inhibition and overexpression of NAMPT gene expression in neutrophils attenuated and aggravated lung injury induced by LPS+MV, respectively, though we have not fully characterized and systematically evaluated their effects yet (FIG. 10).

To test in vivo therapeutic effect, thirty-two wild type C56BL/6 mice with age, gender matched are arranged into 4 groups of 8 mice each. These 4 groups are intravenously injected with NAMPT-shRNA, NAMPT-scramble RNA, NAMPT-cDNA, and NAMPT-antisense cDNA, respectively. 24 hours later, they are subjected to LPS+MV procedure as described in our preliminary study (FIGS. 2-3). At the 24 h point of LPS treatment, BAL and lung tissues of all mice are collected. Various assays in BAL (protein, total and differential cell counts, cytokines TNFα and IL1β) and lung tissues (wet/dry ratio, MPO, cytokines TNFα and IL1β, histological haematoxylin and eosin staining for visualizing alveolar structure, and Masson's Trichrome Staining for Collagen Fibers) are carried out as described by Patel et al. and in our preliminary study (FIGS. 2-3). These parameters inform us whether neutrophil specific NAMPT knockdown or overexpression by NAMPT-shRNA or NAMPT-cDNA will attenuate or aggravate lung injury in LPS+MV mouse model of ALI by comparing all parameters in NAMPT-shRNA or NAMPT-cDNA group with those in scramble or antisense control group Similar procedures will be carried out to test newly synthesized multivalent NAMPT shRNAs.

EXAMPLES X-XIV will investigate the molecular mechanisms underlying the attenuation of CIA in NAMPT knockdown mice.

Neutrophils are the first line of response of the innate immune system to injury, releasing cytotoxic mediators and reactive oxygen species. However, if neutrophils persist, they can induce tissue damage. Therefore, the factors that control the recruitment, function, lifespan and removal of these cells are important for host defense and resolution of inflammation. In RA, macrophages overexpress proinflammatory cytokines, growth factors, histocompatibility complex class II molecules and matrix-degrading enzymes, all leading to increased inflammation, matrix destruction, and angiogenesis. In turn, infiltrating neutrophils and macrophages stimulate SF to proliferate and produce cytokines, chemokines and matrix-degrading enzymes, which ultimately leads to the thickening and progressive destruction of joint membrane, cartilage and bone.

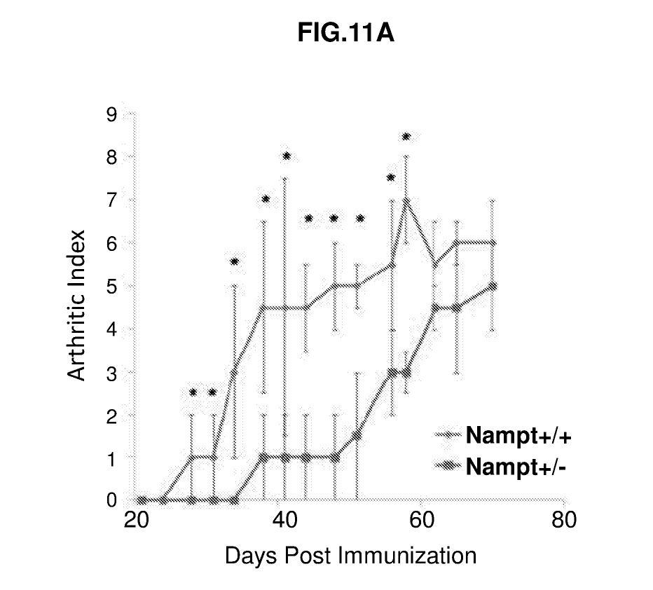

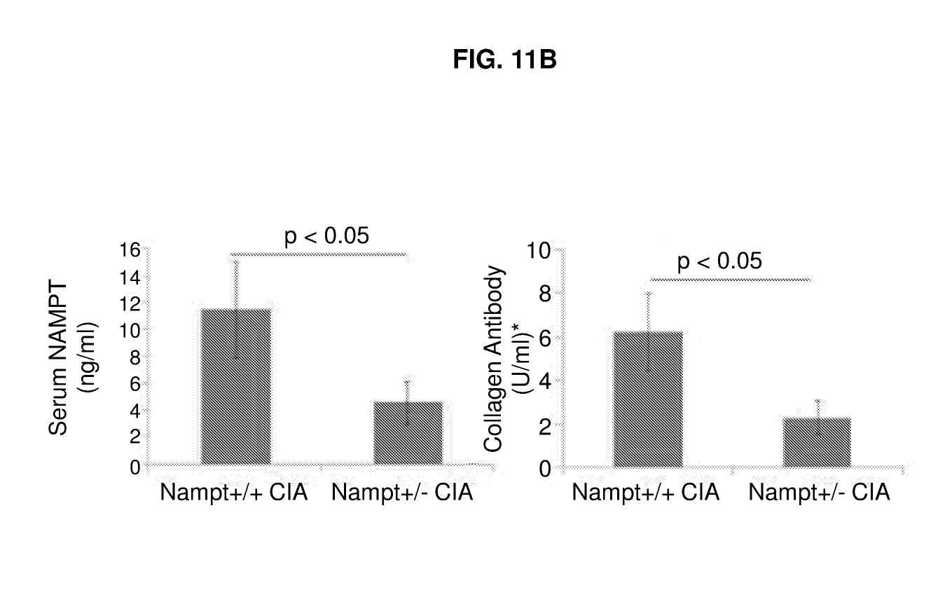

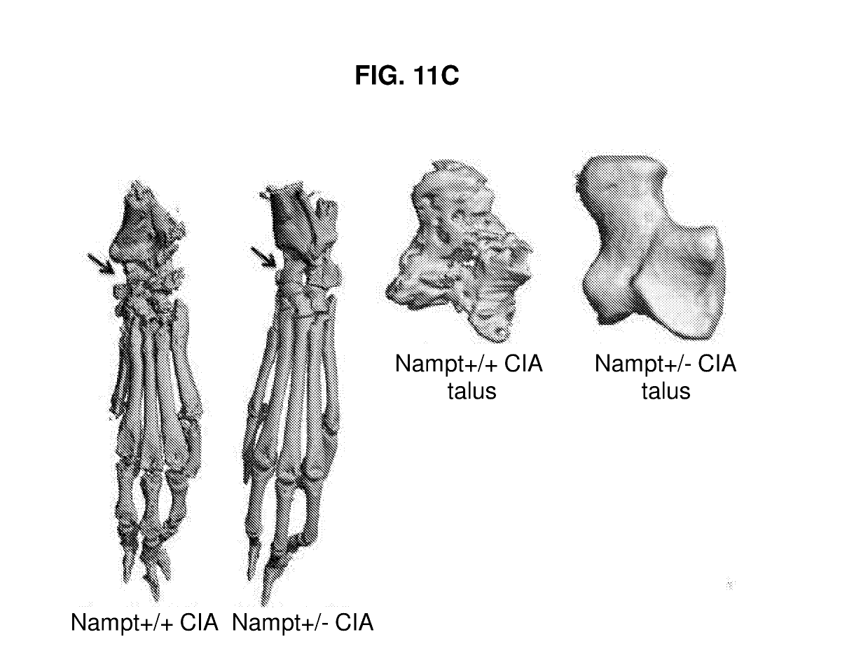

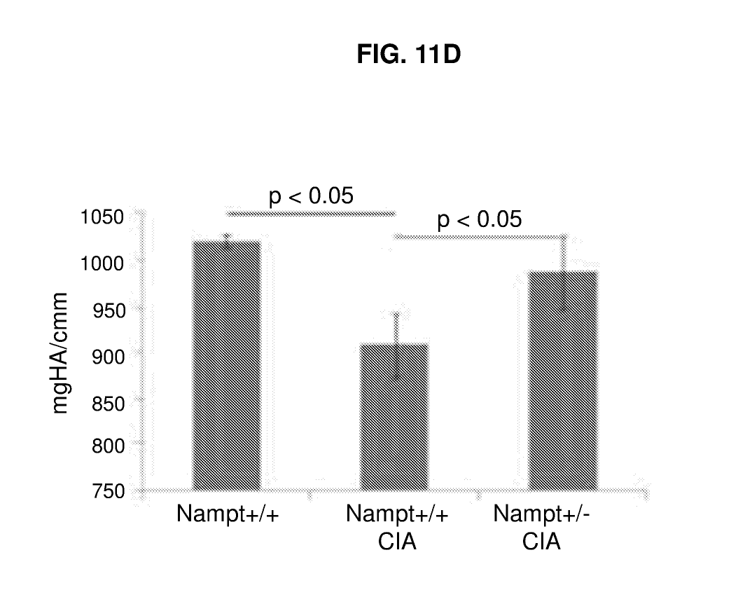

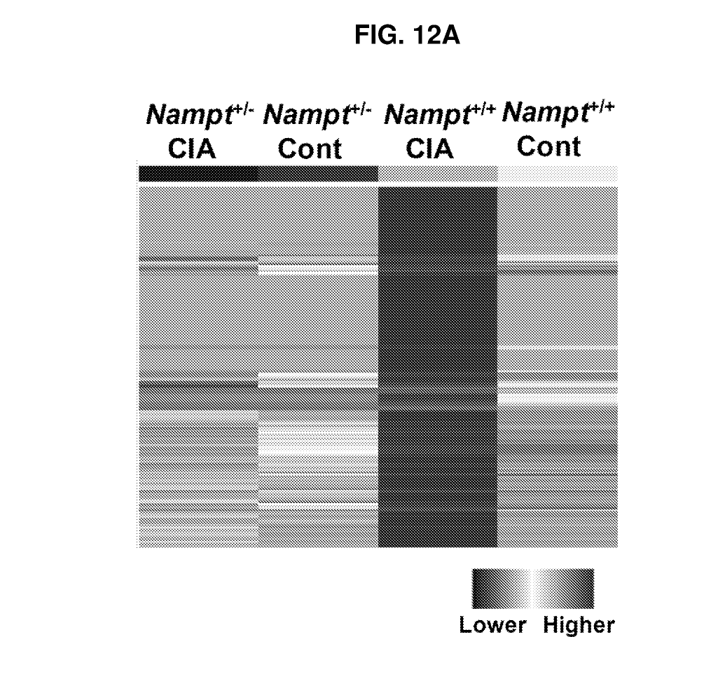

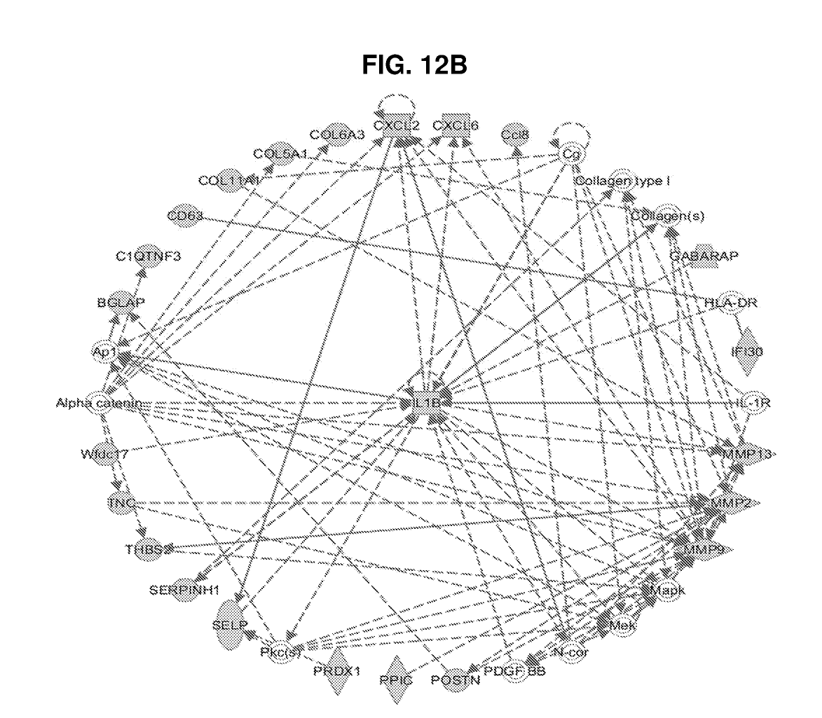

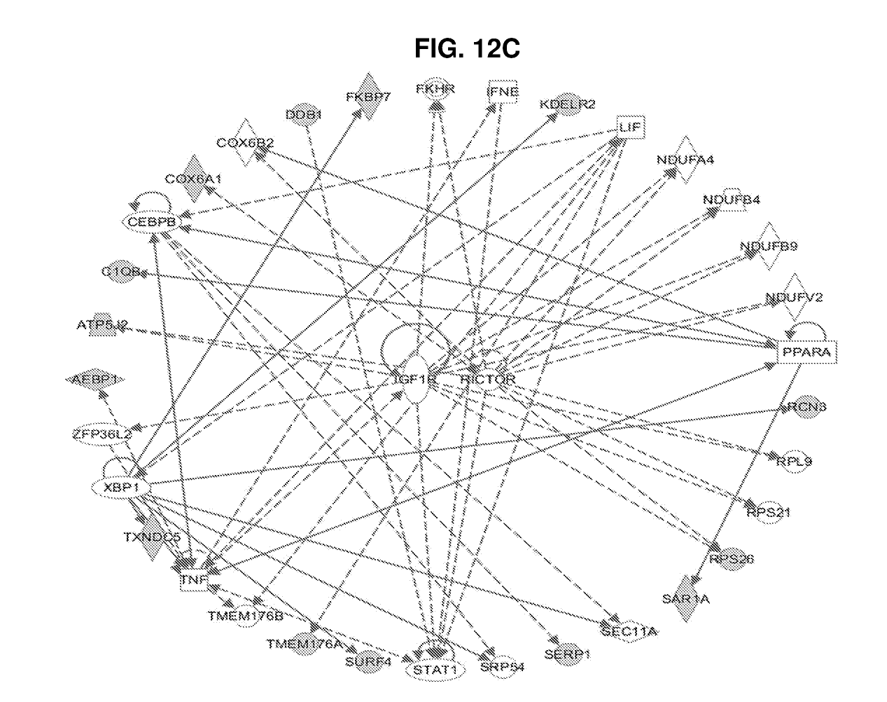

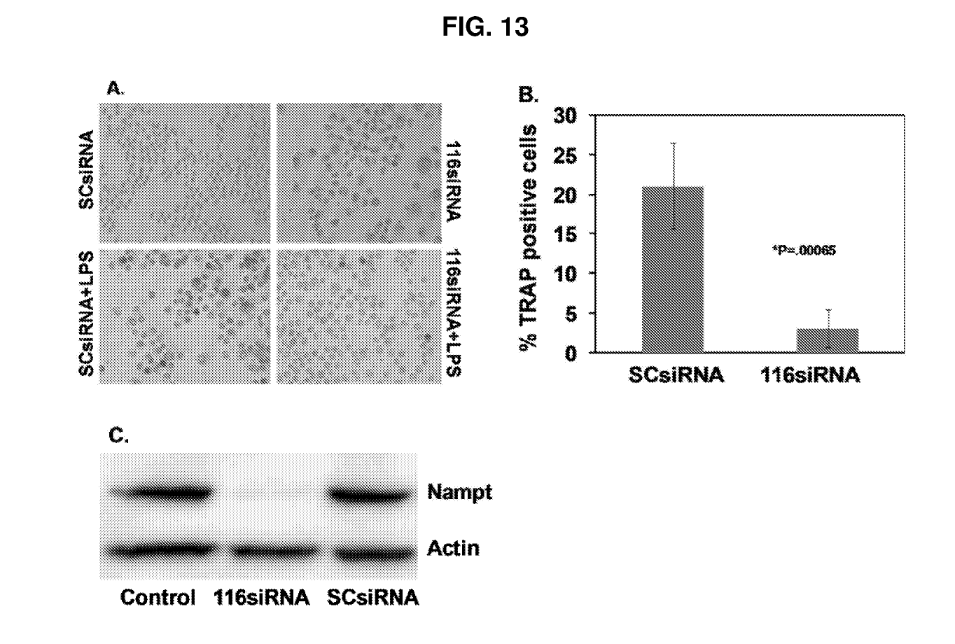

Otero et al., first reported that patients with rheumatoid arthritis had higher plasma levels of NAMPT than healthy controls. The authors proposed that NAMPT could coordinate the inflammatory process in RA, and at the very least could serve as a biomarker for the disease. These findings were confirmed by Nowell et al., Bretano et al., and Matsui et al., Nowell and colleagues detected elevated levels of NAMPT in synovial fluid from RA patients when compared with osteoarthritis (OA) patients. NAMPT expression was immunolocalized within the synovial lymphoid aggregates, which consisted of B cells, T cells, dendritic cells, plasma cells, endothelial cells, macrophage-like synoviocytes, and SF. Bretano et al. demonstrated that NAMPT expression was elevated in the joints of RA patients, especially in the SF localized at the points of invasion into the synovial lining and cartilage. Matsui et al., detected elevated NAMPT mRNA expression in synovial tissue, peripheral blood mononuclear cells, and peripheral blood granulocytes isolated from RA patients compared to healthy controls. Meier et al. demonstrated that recombinant, extracellular NAMPT increased SF motility and cytokine synthesis. The correlation of elevated expression of NAMPT with inflammation and tissue destruction identified NAMPT as an important mediator of the innate immune response and a potential target for RA therapy. These findings suggest that SF play important roles in the initiation and the perpetuation of RA, but the underlying molecular mechanisms are not understood fully. Therefore, to provide a systems approach to uncover the transcriptional regulation in SF we profiled human normal control and RA SF transcriptomes by RNA-seq. We identified 277 genes, 595 known isoforms and 1081 novel isoforms that were differentially expressed in RASF compared to controls, representing key networks and pathways that may contribute to the pathogenesis of RA. In total, these findings prompted us to utilize our NAMPT mouse lines to investigate further the role of NAMPT in the pathogenesis of arthritis. We have generated a congenic DBA/1J NAMPT^{+/-} (N>10) and incipient congenic DBA/1J NAMPT^{OE} mice (currently N=5) by backcrossing to DBA/1J mice. We initiated transferring C57BL/6J NAMPT^{N-/-} mice to the DBA/1J background, since neutrophil specific gene knockdown of Syk is sufficient to block the initiation of arthritis in the K/B×N serum transfer model. Our preliminary results demonstrated clearly that mice expressing lower levels of NAMPT (NAMPT^{+/-}) present with a decreased auto-immune response and are protected against bone erosion compared to their wild-type littermate controls (NAMPT^{+/+}) after arthritic induction by collagen injection (FIG. 11). We monitored joint inflammation twice per week for 70 days (FIG. 11A). The progression of inflammation was less severe in NAMPT^{+/-} mice compared to NAMPT^{+/+} littermate controls. Serum levels of anti-mouse CII antibody and NAMPT demonstrated the decreased immune response corresponded with decreased levels of circulating NAMPT (FIG. 11B). MicroCT analysis revealed bone erosion of the talus of both NAMPT^{+/+} and NAMPT^{+/-} mice with CIA, but the erosion was milder in the NAMPT^{+/-} mice. The average bone volume of the talus in CIA was greater in NAMPT^{+/-} compared to NAMPT^{+/+} mice. RNA-seq analysis of whole ankle joints identified 171 genes uniquely upregulated in arthritic NAMPT^{+/+}, but not in NAMPT^{-/-} CIA mice, demonstrating further the protective nature of NAMPT knockdown (FIG. 12A). The top two networks identified by IPA (FIGS. 12 B, C) contain 24 genes not previously linked to arthritis. NAMPT is also required for the differentiation of RAW264.7 macrophages into osteoclasts (FIG. 13). Therefore, these Examples test the hypothesis that NAMPT mediates inflammatory response and tissue destruction in multiple cell types, including SF, macrophages, and neutrophils.

### Example X

### Determination of Mechanisms by which NAMPT Mediates Progression and Severity of Collagen Induced Arthritis

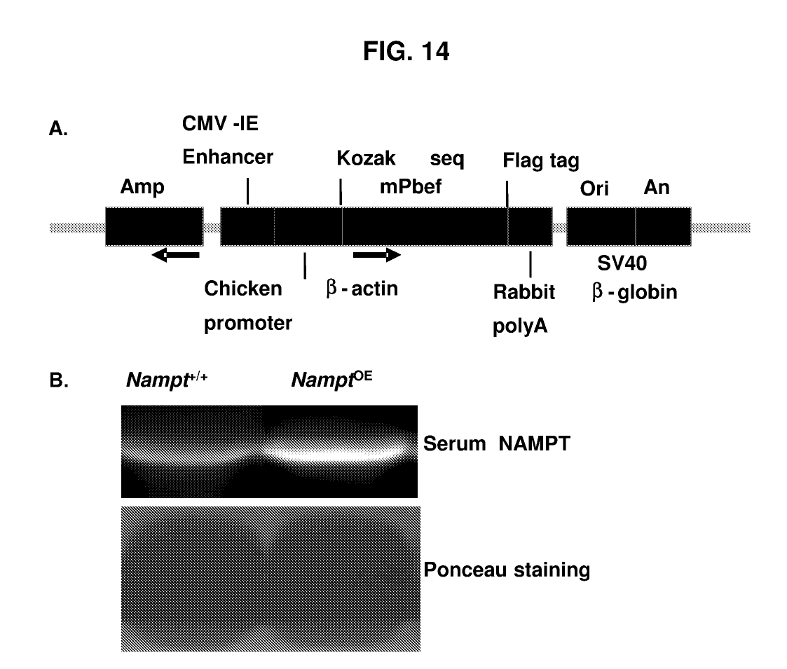

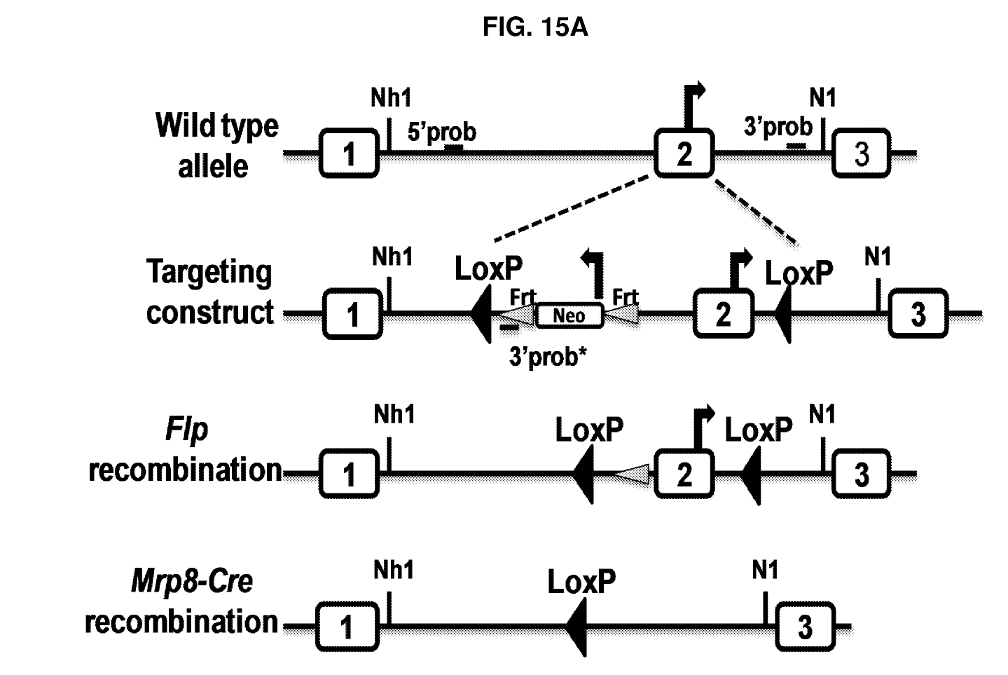

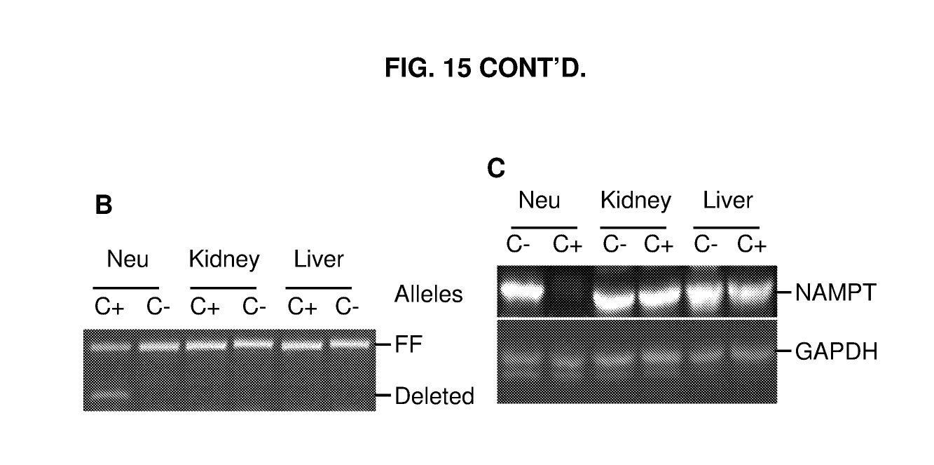

We examine CIA in NAMPT^{+/+}, NAMPT^{+/-}, NAMPT^{OE} (FIG. 14), NAMPT^{N-/-} (FIG. 15) mice to determine how NAMPT mediates the inflammatory response and tissue destruction. Tissue is isolated at 49 days based upon our preliminary results showing significant difference in the arthritic index between NAMPT^{+/-} and NAMPT^{+/+} mice from day 28 to 58 (FIG. 11A). To induce arthritis, mice are immunized with an emulsion of type II bovine collagen (Chondrex; 100 μg) with Complete Freund's Adjuvant (Chondrex; 100 μg M. tuberculosis) by intradermal injection at the base of the tail. A booster of collagen (100 μg) in IFA (Chondrex) is administered at day 21. Untreated mice serve as baseline controls. Male mice, 8 weeks old, 14 mice per group, are divided into 8 groups: A. NAMPT^{+/-}+collagen; B. NAMPT^{+/+}+collagen; C. NAMPT^{OE}+collagen; D. NAMPT^{N-/-}+collagen; E. NAMPT^{+/-}; F. NAMPT^{+/+}; G. NAMPT^{OE}; H. NAMPT^{N-/-}. These groups allow analyses between genetically modified groups (e.g. NAMPT^{+/+} vs. NAMPT^{+/-}) and within genetically identical groups (e.g. NAMPT^{+/-}+collagen vs. NAMPT^{+/-}). A sample size of n=14 was calculated for each group using ANOVA Sample Size of SigmaPlot (Ver. 13, Systat Software, Inc., San Jose, Calif.) based on 8 groups, the Minimum Detectable Difference in Means: 0.15, Expected Standard Deviation of Residuals: 0.100, Desired Power: 0.85 and Alpha value: 0.05. Within 16-25 days, ˜90% of the mice develop arthritic symptoms that closely mirror the symptoms of RA. Mice are evaluated for the onset of inflammation and scored (scale 0-4 per paw) every 3-4 days for 49 days. Serum is collected for measurement of circulating expression levels of NAMPT, SAA, and anti-collagen antibodies using ELISAs. Synovial tissue is removed for histology, immunohistochemistry, and gene expression analyses. The right paw is collected for SF isolation and gene expression analyses. The left paw is examined morphologically by microCT and histological analyses for inflammation and bone erosion. Paraffin sections are stained with H&E and Safranin O. Tartrate resistant acid phosphatase staining with methyl green counterstaining is performed to visualize TRAP⁺ osteoclasts. Histomorphometric measurements are performed using OsteoMeasure software (OsteoMetrics). Immunohistochemistry on paraffin sections is completed initially using NAMPT, TNFα, and β-actin antibodies. SF is collected according to the procedure of Armaka et al., and cultured for RNA and protein isolation. Bone marrow derived macrophages are isolated and cultured as described in Mukai et al., and Ueki et al. Neutrophils are isolated from bone marrow by MACS separation using LS columns (Miltenyi Biotec, Cat. No: 130-042-401). RNA-seq (Illumina TruSeq; 6.2 GB, 104×) are performed on primary SF (passage 3; these cells retain their initial P1 phenotype), macrophages (P1), and neutrophils (P1). (N=3 per experimental group). Data are analyzed by a Tuxedo suite pipeline, modified for transcript assembly with StringTie. Differentially expressed transcripts are subjected to pathway analysis using IPA and DAVID. Novel therapeutic targets identified by RNA-seq and pathway analyses are validated by qPCR and western blot analyses, followed by functional experiments to determine NAMPT mediated mechanism(s).

### Example XI

### Determination of Signal Transduction Cascades Associated with CIA in SF

We test the hypothesis that differential expression of NAMPT alters the signal transduction cascades regulating the production of proinflammatory cytokines and matrix degrading enzymes of SF. To elucidate further the NAMPT mediated signal transduction cascades, we utilize SF isolated from the NAMPT^{+/+}, NAMPT^{+/-}, and NAMPT^{OE} mice in either the normal or arthritic state for in vitro cell assays. Pure cultures of SF (N=4 per mouse line) are isolated from hind ankle joints according to the method of Armaka et al. The cells are used between P3-P7. To extend their utility, primary SF is immortalized by nucleofection (4D-Nucleofector™ System, Lonza) with pBABE-puro-hTERT (Addgene #1771). To date, we have immortalized NAMPT^{+/+} and NAMPT^{+/-} mouse SF. A panel of primary and immortalized SF with NAMPT^{+/+}, NAMPT^{+/-}, and NAMPT^{OE} genotypes afford the ability to perform gain- and loss-of-function experiments without transfection. However, as needed we nucleofect (P2 solution, program EN-150) SF with NAMPT overexpression and knockdown vectors (FIG. 9C). 3T3-L1 (ATCC® CL-173™) cells are used as an experimental control. Because the arthritic joint is hypoxic and rich in proinflammatory cytokines, experiments are completed under normoxic and hypoxic conditions in the presence or absence of stimulation (e.g. TNFα±IL1β). NAMPT, IL1β, IL-6, Cxcl15 (IL-8), TNFα, RANKL, VCAM-1, and Hif1α expression are measured. We include the expression of genes and proteins associated with pathways identified in RNA-seq analyses performed in EXAMPLE X. Gene expression is measured by qPCR (TaqMan® Assays). Protein expression is measured by western blot. Cytokine levels are measured by either ELISA, multiplexed analysis (Luminex 200), or flow cytometry. RNA-seq analyses on cultured SF are performed as needed. As we elucidate signal transduction cascades, we employ inhibitors (if available) to disrupt the pathways for further analyses. For example, the signal transducer and activator of transcription 3 (STAT3) pathway, which is activated in RA, can be blocked by treatment with S3I-201, a STAT3 phosphorylation inhibitor. This Example allows us to identify and characterize the arthritis-associated networks controlled by NAMPT.

### Example XII

### Determination of Functional Differences Between Macrophages Isolated from CIA Mice

This experiment tests the hypothesis that differential expression of NAMPT in macrophages alters their ability to differentiate into osteoclasts. We examine the differentiation of bone marrow derived macrophage isolated from NAMPT^{+/+}, NAMPT^{+/-}, NAMPT^{OE} mice in either the normal or arthritic state (N=4 per mouse line, condition). To extend their utility, primary macrophages are immortalized by as described in EXAMPLE X1. Transfected RAW 264.7 (ATCC® TIB-71™) cells are used as an experimental control. Bone marrow derived macrophages are isolated, cultured, and differentiated as described in Mukai et al., and Ueki et al.

### Example XIII

### Determination of Functional Differences Between Neutrophils Isolated from CIA Mice

This experiment tests the hypothesis that differential expression of NAMPT in neutrophils alters their chemotactic migration, activation and apoptosis. We examine the following functions using bone marrow derived neutrophils isolated from NAMPT^{+/+}, NAMPT^{+/-}, NAMPT^{OE}, NAMPT^{N-/} mice in either the normal or arthritic state (N=4 per mouse line, condition).

Chemotaxis of neutrophils towards known chemoattractants (e.g. FMLP, NAMPT) is be carried out using The CHEMICON® QCM™ Chemotaxis 3 μm 24-well Migration Assay in a Migration Chamber, based on the Boyden chamber principle (Cat. No. ECM505, EMD Millipore). Migrating cells will be lysed and detected by the CyQUANT® GR dye (Molecular Probes).

Activation of neutrophils is assessed by measurement of neutrophil shape change and by CD62-L/CD11b expression levels by qPCR. Superoxide anion production is assessed by dihydrorhodamine fluorescence. Neutrophil TNFα and IL1-β levels are measured by ELISA.

Apoptosis is assessed by flow cytometric analysis for Annexin-V/propidium iodide staining and by analysis of cleaved caspase-3 by western blot and ELISA.

Phagocytosis is measured with pHrodo® Red S. aureus Bioparticles® Conjugate for Phagocytosis (Cat. No., A10010, Thermo Fisher Scientific Inc.) by flow cytometry and fluorescence microscopy.

### Example XIV

### Determination of Crosstalk Between SF, Macrophages and Neutrophils in CIA

This experiment tests the hypothesis that the interplay of SF with macrophages and neutrophils exacerbate the progression and severity of arthritis. We examine the following interactions between SF, macrophages and neutrophils isolated from NAMPT^{+/+}, NAMPT^{+/-}, NAMPT^{OE}, NAMPT^{N-/-} mice in either the normal or arthritic state (N=4 per mouse line, condition). We test all possible cell to cell combinations (e.g. NAMPT^{N-/-} neutrophil vs. NAMPT^{OE} SF).

Chemotaxis of neutrophils or macrophages towards SF conditioned media is carried out using The CHEMICON® QCM™ Chemotaxis 3 μm 24-well Migration Assay in a Migration Chamber as described in Experiment XIII

Synovial fibroblast motility is measured by electric cell-substrate Impedance sensing (ECIS® Zθ; Applied Biophysics, Troy, N.Y.) to quantify cell behavior. We measure SF motility upon exposure to either conditioned media or direct cell to cell contact with neutrophils and macrophages using both wound healing and electric fence procedures. We test all genotypic combinations of the cells in both normoxic and hypoxic conditions.

Signal transduction is detected by changes of resistance (ECIS® Zθ) when the SF are exposed to either conditioned media or directly to neutrophils and macrophages. We also test the effect of increasing doses of TNFα±IL-1β on changes in resistance (signal transduction) in SF. We test all genotypic combinations of the cells in both normoxic and hypoxic conditions.

Gene expression in cultured SF exposed to conditioned media from cultures of neutrophils or macrophages is measured by qPCR and western analyses. The supernatant (media) is collected and cytokine expression is detected by ELISA and/or Lumenix assays. Media alone (no exposure to SF) will serve as the control.

Statistical analyses. To evaluate differences in the variables between NAMPT^{+/+}, NAMPT^{+/-}, NAMPT^{OE}, NAMPT^{N-/-} groups, parametric statistical analyses are performed using Sigmaplot analysis software with an unpaired t-test (two groups) or ANOVA (multiple groups) followed by the Tukey-Kramer Multiple Comparisons posttest. Results are represented as mean±SD. Two-tailed P values <0.05 are considered significant.

Examples XV-XVII evaluate the efficacy of anti-NAMPT therapies in CIA.

Treatment of RA is problematic because individual RA patients differ in both their genetic background and the progression of the disease. However, the genetic component of the disease provides a significant opportunity for the identification of new biological factors, like NAMPT, that can be targeted for therapeutic intervention. We determine the ability of three novel therapies to inhibit NAMPT activity and/or expression in vivo and to attenuate CIA. We developed the NAMPT inhibitor, MC4-PPEA (FIG. 6). We also constructed four-copy NAMPT-shRNA (4CshRNA) expression vectors (FIG. 9). In addition, we have cloned anti-NAMPT single-chain variable (scFv) antibody expression vectors (FIG. 1). MC4-PPEA, 4CshRNA, and scFv will be administered to DBA/1J wild-type (NAMPT^{+/+}) mice at specific time points following collagen injection. To determine the therapeutic benefit, including the appropriate dose of each therapy, mice are evaluated for the onset and progression of arthritis. The mice are characterized for inflammation, bone erosion, and gene expression. We modify our novel inhibitors as needed to increase their efficacy in attenuating CIA. We demonstrate the ability of MC4-PPEA, 4CshRNA, and scFv to attenuate CIA.

The dysregulation of NAMPT activity in RA makes it an attractive target for therapeutic intervention. NAMPT has been demonstrated to be a key player in inflammatory arthritis. CIA is accompanied by increased expression of NAMPT in both serum and the arthritic paw. Administration of FK866, a competitive inhibitor of NAMPT, has been shown to effectively reduce the severity and progression of arthritis, while a liposome-packaged NAMPT siRNA delivered by tail vein injection has been shown to attenuate the immune response in mice by lowering the number of circulating monocytes and decreasing serum levels of inflammatory cytokines. Inhibition by FK866 in CIA mice has provided strong evidence that NAMPT is a promising therapeutic target. However, screening for additional inhibitory molecules is needed, as thrombocytopenia is a potential side effect of FK866 treatment in humans. Therefore, we replace the benzoylpiperidine moiety of FK866 with a carborane moiety. This supercharged FK866 molecule, termed MC4-PPEA exhibits a 100-fold increase in NAMPT inhibition compared to FK866. Furthermore, the half-maximal inhibitory concentrations (IC50) are about 10 fold lower than FK866 in several cell lines tested. MC4-PPEA is more effective than FK866 in preventing the TNFα induced nuclear translocation of NF-kβ and trans-endothelial resistance (FIG. 7). In addition to small molecule inhibitors, scFv antibodies have been successfully applied as diagnostic reagents and therapeutic gene deliveries. siRNA or shRNA-based therapeutics have also demonstrated the capability to silence therapeutically relevant genes in various in vivo models of cancer, infections autoimmune diseases and other genetic disorders including CIA. In experiments XV-XVII we improve the forms of M4-PPEA, NAMPT-4CshRNA, and NAMPT-scFv to develop powerful NAMPT tools as a viable therapeutic strategy.

### Example XV

### Evaluation of Therapeutic Efficacy of NAMPT Targeted Small Chemical Inhibitors in CIA

To determine the therapeutic efficacy of MC4-PPEA in attenuating arthritis, we first optimize the structure in terms of the placement of the carborane moiety (FIG. 15) and its water solubility. Our labs have produced a derivative bearing a hydroxymethyl moiety on the C-7 carbon atom of the cage (hm-MC4) (FIG. 6). This functionality serves as the site for the covalent attachment of water solubilizing groups, such as a methoxypolyethylene glycol chain, or a sulfate ester. Potent “next generation” NAMPT inhibitors bearing a sulfone moiety have recently been reported. We incorporate this moiety into the MC4 structure and investigate its effects on inhibitory activity and solubility. The activity of these new molecules is measured initially using NAMPT inhibitory assays in vitro. We then test promising MC4-PPEA derivatives to modify cytokine expression and inhibit enzyme activity using in vitro assays in primary mouse SF. The results of the in vitro assays inform the design of the in vivo assay. We test further the dose and time of treatment by administering MC4-PPEA derivatives to wild-type mice to determine the optimum conditions for inhibiting NAMPT activity in vivo. Based upon these results, we initially analyze five groups of mice: A. DBA/1J; B. DBA/1J+vehicle; C. DBA/1J+FK866; D. DBA/1J+MC4-PPEA (1); DBA/1J+MC4-PPEA (2). A sample size of n=13 for each group is calculated using ANOVA Sample Size of SigmaPlot based on 5 groups, the Minimum Detectable Difference in Means: 0.15, Expected Standard Deviation of Residuals: 0.100, Desired Power: 0.85 and Alpha value: 0.05. Male mice, 8 weeks old, 13 mice per group are immunized with collagen, weighed, scored and analyzed. FK866 (10 mg/kg) and two different MC4-PPEA derivatives based upon our in vitro assay, along with the vehicle control, are administered at the first sign of paw inflammation (˜day 21) and continued twice a day for 15 days. Animals are euthanized for tissue isolation at the completion of treatment. Serum markers, histology, microCT imaging, and gene expression are all examined. Blood is analyzed by flow-cytometry and VetABC to characterize changes in cell profiles and to determine if MC4-PPEA treatment alters the number of circulating platelets. Based upon the cumulative results of the initial experiment, we adjust the doses and lengths of treatment as needed. qPCR and western blot assays are performed on neutrophils, macrophages, and SF isolated from MC4-PPEA treated and control treated mice. RNA-seq is be performed to characterize the mechanism of attenuation once we have identified the most effective dose and treatment conditions. As we characterize the role of neutrophils, macrophages, and SF in the progression and severity of CIA, we modify MC4-PPEA further. For example, if we find in that NAMPT^{N-/-} mice are protected against CIA, we develop a neutrophil-specific NAMPT inhibitor by covalently linking the optimized hydroxymethyl MC4 compound to a neutrophil targeting peptide, such as GGPNLTGRW (GGP). To produce these new conjugates, we utilize click chemistry using a short azide terminated PEG linker and a peptide bearing an alkyne (FIG. 6). The binding avidity of the new conjugates for targeted tissue are measured in vitro using competition assays and blocking studies with fluorescent analogs. Neutrophil specific knockdown will be confirmed by qPCR, western blot, neutrophil function using macrophages and SF as controls. A control peptide whose sequence was generated from a scrambled GGP peptide sequence is coupled to MC4-PPEA and used as both in vitro cell binding and in vivo neutrophil targeting controls.

### Example XVI

### Evaluation of the Therapeutic Efficacy of NAMPT Targeted shRNA in CIA

To determine the therapeutic efficacy of NAMPT-4CshRNA expression vectors in attenuating arthritis, we first transfer the 4 copy shRNA constructs (FIG. 9B) (pENTR™/TOPO-D backbone) into pLenti6.4/R4R2/V5-DEST using Gateway®-Recombination Cloning Technology (Life Technologies). We use Life Technologies' ViraPower HiPerform Lentiviral Packaging technology (cat# K4975-00) to generate high titer, replication-incompetent lentivirus expressing the four NAMPT shRNA molecules. We test the ability of our lentiviral constructs to modify NAMPT expression and cytokine expression using in vitro assays in primary mouse SF. We optimize the length and dose of LV (MOI) required to knockdown NAMPT expression (>50%) by measuring NAMPT mRNA (TaqMan®) and protein (western blot) following tail vein injection of packaged virus. Once the optimum length and dose have been determined, we initially analyze five groups of mice: A. DBA/1J; B. DBA/1J+vector; C. DBA/1J+scrambled vector; D. DBA/1J+4CshRNA-identical DBA/1J+4CshRNA-unique. A sample size of n=13 for each group is calculated using ANOVA Sample Size of SigmaPlot based on 5 groups, the Minimum Detectable Difference in Means: 0.15, Expected Standard Deviation of Residuals: 0.100, Desired Power: 0.85 and Alpha value: 0.05. The effects on CIA are evaluated accordingly. We modify the vectors to include inducible or tissue specific promoters. We have constructed a neutrophil specific adenoviral shRNA expression vector (FIG. 9A). We also test an alternative method to deliver our shRNA. The cationic liposome RPR209120/DOPE has been used for RNAi silencing in CIA, thus we package our corresponding siRNA in the liposome and administer by IV injection into CIA mice, following the same experimental design established for the LV delivery of the 4CshRNA. Subsequently, we compare the effectiveness of the two delivery methods (LV and liposome) to attenuate CIA.

### Example XVII

### Evaluation of the Therapeutic Efficacy of NAMPT Targeted scFv in CIA

We expressed and purified recombinant mouse NAMPT protein and obtained two human against mouse NAMPT antibody gene clones, termed NAMPT-scFv1 and -scFv2, by screening a HuScL-2™ Phage Display Naïve Human scFv Library. We cloned the cDNAs for each scFv, including a histidine-tag on the 3′ end, into the pCAGGS expression vector. The V_{H} and V_{L} fragments for each scFv are separated by a (G4S)3 linker and encode proteins of 247 and 249 amino acids, respectively for NAMPT-scFv1 and -scFv2 (FIG. 1). To increase the specificity of these constructs we generate hetero-dimer and hetero-tetramers using established procedures. The hetero-dimer [scFv1-scFv2], also called a di-body, is separated by a GGSSRSSSSGGGGSGGGG linker. The hetero-tetramer, also called a tetra-body, consists of two dimers [scFv1-scFv2]₂ joined by the HIV1 P21 capsid protein linker, GATPQDLNTML. The P21 linker is an epitope for the murine monoclonal CB4-1 antibody. Similar to the mono-bodies (scFv1 and scFv2), the dibody and tetrabody possess a C-terminal His-Tag. We utilize the CB4-1 antibody and nickel-columns (His-Tag) to manipulate and purify our recombinant scFv antibodies. We enhance the specificity and tertiary structures of the dibody and tetrabodies by modifying the type and length of the linker sequences. The antisense sequence of NAMPT-scFv1 and -scFv2 are cloned and used as controls. Similar procedures are carried out to test newly synthesized dibody and tetrabodies. The most effective constructs are cloned into a Lentiviral vector using Gateway® Recombination Cloning Technology (Life Technologies) and optimized by testing in 3T3-L1 and RAW264.7 cells. We test optimized constructs and doses in primary SF and wild-type mice. We test the ability of our top constructs (di- or tetra-) to attenuate CIA, by initially analyzing 7 groups of mice: A. DBA/1J; B. DBA/1J+LV vector; C. DBA/1J+LV-scFv-scrambled; D. DBA/1J+LV-scFv1, E. DBA/1J+LV-scFv2, F. [scFv1-scFv2]₁, G. DBA/1J+LV-[scFv1-scFv2]₂. A sample size of n=14 for each group is calculated using ANOVA Sample Size of SigmaPlot based on 7 groups, the Minimum Detectable Difference in Means: 0.15, Expected Standard Deviation of Residuals: 0.100, Desired Power: 0.85 and Alpha value: 0.05. The effects on CIA are evaluated accordingly, following the experimental design in ED2-1.

### Statistical Analyses.

Parametric statistical analyses is performed using Sigmaplot analysis software as described in Experiment X.

### Results.

These experiments demonstrate the efficacy of attenuating arthritis with novel anti-NAMPT reagents, including MC4-PPEA, 4CshRNA, and scFv, in a CIA mouse model.

EXAMPLES XVIII-XXI characterize functionally the human NAMPT gene promoter.

These experiments test the hypothesis that SNPs within the NAMPT promoter exert differential allelic effects on expression, which underlies susceptibility to arthritis. Our preliminary data indicate that SNPs within the NAMPT promoter, and their corresponding haplotype combinations, are associated with JIA. To determine the functional consequences of each promoter haplotype Luciferase reporter assays in SW982 (ATCC® HTB-93™) human synovial fibroblasts are used to determine both allele and haplotype effects of the four promoter SNPs on NAMPT expression. We validate the luciferase assays by CRISPR/Cas9 genome editing of the NAMPT promoter in SW982 cells to generate the key haplotypes followed by measurement of NAMPT mRNA and protein expression. EMSA is performed to assess initially whether transcription factors (TFs) bind the SNP alleles differentially. A modified (B1H) is used to isolate potential TFs. As needed, we also employ traditional affinity chromatography, supershift assays, and ChIP qPCR methods to identify and isolate TFs. Finally, we utilize the CRISPR/Cas9 system to humanize the regulation of the mouse NAMPT gene by replacing the mouse NAMPT promoter with a panel of protective and susceptible human NAMPT promoters. A humanized NAMPT promoter mouse model allows a systems approach to elucidate the regulation of NAMPT in multiple tissues and cell types upon induction of arthritis. We characterize the role of the protective and susceptible haplotypes, and their corresponding TFs, in the regulation of NAMPT transcription in arthritis

During our previous investigation of NAMPT in acute respiratory distress syndrome, we published the identity of 11 SNPs in the NAMPT gene promoter. Our study was followed by several genetic association studies linking these SNPs with disease. Since no one has analyzed the NAMPT promoter SNPs in JIA, we collaborated to genotype 4 SNPs (G-1535A, A-1001C, C-948A, T-423C) in a cohort of JIA patients. The minor alleles for 3 SNPs (−1001, −948, −423) are protective compared to the controls (P<0.05; (OR=0.6, 0.2, 0.77, respectively). Haplotypes were estimated with H-Plus. GACT is associated slightly with susceptibility in JIA patients compared with controls: OR=1.23 (1.05-1.45) p=0.01, GCAC is a significantly protective haplotype OR=0.51(0.30-0.87) p=0.01. We found the GCCC haplotype associated with severity (p=0.026, OR=6.2; 1.25-30.7)). These results suggest that NAMPT SNP alleles and their haplotypes are associated with the susceptibility to JIA. We hypothesize that the susceptible and protective haplotypes are associated with an increase and decrease in NAMPT expression, respectively.

### Example XVIII

### Determination of Differential Allelic and Haplotype Effects on Gene Expression for 4 NAMPT Promoter SNPs in SF

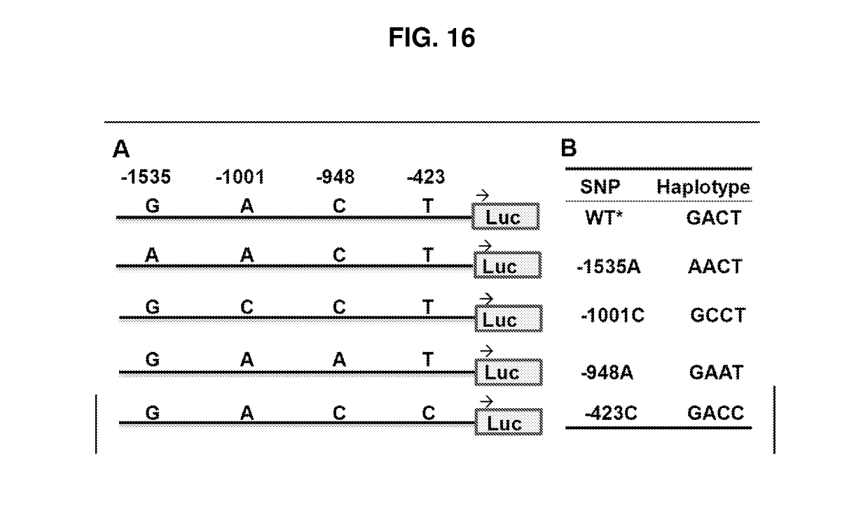

This experiment was designed to test the hypothesis that SNPs within the NAMPT promoter exert differential allelic effects on expression, which underlies susceptibility to arthritis by performing Dual-Glo® luciferase gene reporter assays (Promega, Madison, Wis.). To correlate the SNPs with the transcriptional activity (strength) of the NAMPT promoter, we have generated 16 plasmids containing the 16 possible haplotype combinations for the 4 SNPS (2⁴) within the 1974 bp full length NAMPT promoter cloned into the pGL3-Basic firefly reporter (Promega) (FIG. 16). These plasmids allow us to elucidate the effect of changing a single or multiple SNP(s) in the context of the full promoter. Human SW982 (ATCC® HTB-93™) SF is be transfected (Lipofectamine® 2000; Life Technologies, Grand Island, N.Y.) with the reporter plasmids cultured in normoxic and hypoxic conditions ±TNFα stimulation prior to the luciferase assay. Renila luciferase reporter is cotransfected for the normalization of transfection efficiency. All assays are compared to the control plasmid which contains the major haplotype GACT. We test selected haplotypes in a second human SF cell line, MH7A (Riken BRC RCB1512), which informs us if the observed regulation the NAMPT promoter is unique to SW982 cells or more generalized to SF. We test selected haplotypes in both undifferentiated and PMA-differentiated human THP-1 (ATCC® TIB-202™) and human HL-60 (ATCC® CCL-240™) cells to represent macrophages and neutrophils, respectively. Luciferase reporter assays provide a high throughput method to characterize multiple NAMPT promoter variants under many different conditions. However, it is still not a direct measurement of the promoter on NAMPT expression. We validate our findings by directly mutating the SNPs in the NAMPT promoter in SW982 cells by CRISPR/Cas9 genome editing, thus instead of measuring luciferase activity, we directly measure the effects of the SNPs (haplotypes) on NAMPT mRNA and protein expression. We have adapted the CRISPR/Cas9 technology to study the importance of various SNPs in the NAMPT promoter using the methods of Ran et al., and Wang et al., for homology directed repair (HDR). Initially we utilize HDR for whole promoter replacement with px459 plasmid (Addgene #48139), a guide RNA targeting the NAMPT promoter, and a PCR product containing the new version of the NAMPT promoter. We generate single SNP mutations using smaller donor templates. To “bash” the promoter further, we begin a systematic deletion derivative strategy to analyze smaller promoter elements to elucidate the effect of each SNP and the interactions between the SNP alleles on the transcription of NAMPT. By analyzing the haplotype combinations of the SNP alleles, we acquire information on the interplay between regulatory elements within the NAMPT promoter. We test all constructs initially in SW982 cells, followed by validation of select constructs in MH7A, THP-1, and HL-60 cells.

### Example XIX

### To Determine Differential Allelic Effect of Each of 4 SNPs on Binding Affinities of Potential Transcription Factors in Synovial Fibroblasts

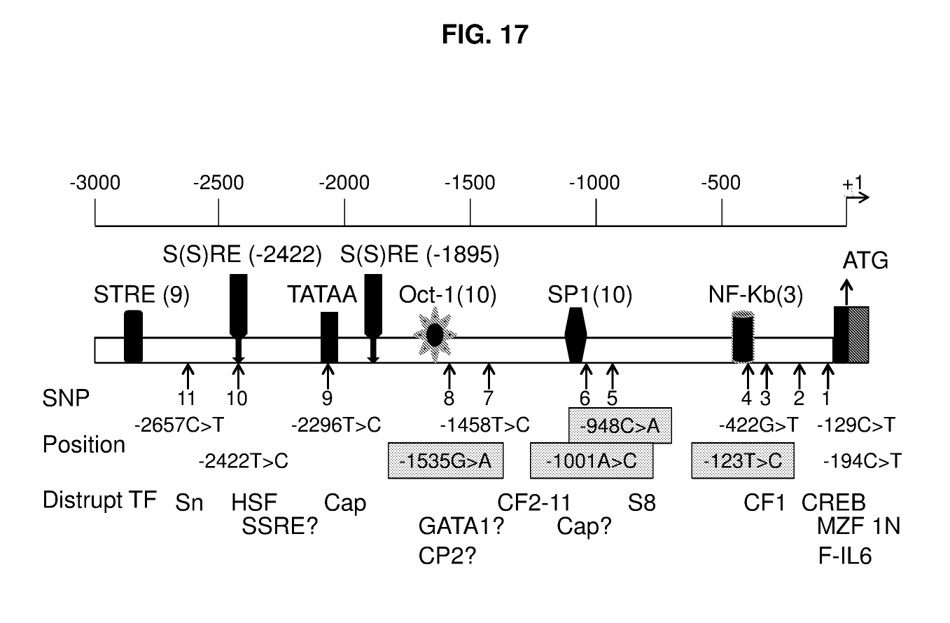

This example is designed to establish that there are differential allelic effects of the 4 SNPs on binding affinities of potential TFs in synovial fibroblasts. This is partly based on in silco prediction (FIG. 17) and our previous findings. Strong evidence that −1535G>A lies within a TF binding site was revealed by EMSA comparing the binding affinities of probes containing either of −1535 alleles for nuclear extracts from human pulmonary artery endothelial cells (HPAEC). Our findings have been seconded in HUVEC cells. To test this observation in SF we perform the EMSA assay according to our previously established method using the LightShift chemiluminescent EMSA kit (Pierce, Rockford, Ill.) to assess whether nuclear proteins extracted from SW982 cells differentially bind the 4 SNP alleles. We include the A-1001C, C-948A and T-423C SNPs. Biotin-labeled 28mer oligonucleotide probes containing either the major allele or minor allele in the 15^{th} position are utilized to determine TF binding affinities. Nuclear extracts are isolated from human SW982 cells cultured under normoxic and hypoxic conditions. Binding specificity is verified by the competition of excess unlabeled cognate probes. Bioinformatic analyses, including TF binding site prediction and data generated by the Encode project, are performed to seek clues on potential TF binding. If a known TF is suspected to bind a SNP site and its antibody is available, supershift and CHiP qPCR assays are performed to provide confirmatory evidence. By comparing the binding band intensity between the major allele and minor allele containing probes in both normoxic and hypoxic conditions in the EMSA, we determine whether there are differential allelic effects of the SNPs on TF binding affinity in SF. As needed we validate are findings in nuclear extracts from MH7A cells.

### Example XX

### Identification of Potential Transcription Factors Binding to SNP Containing Sites in SF

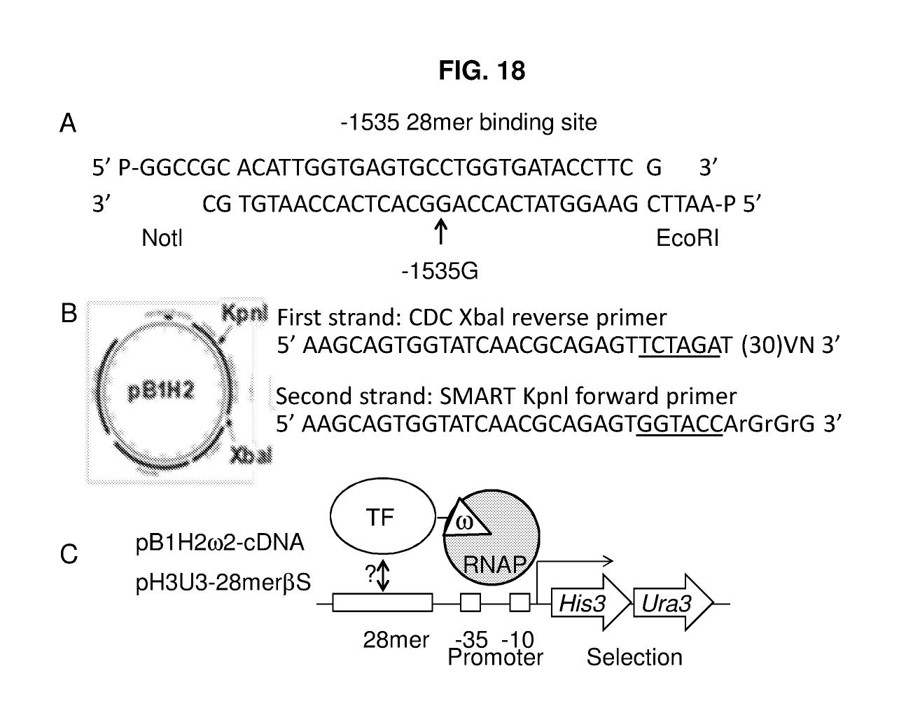

This example is designed to test the hypothesis that each SNP exerts differential allelic effects on NAMPT expression by altering its binding affinity of potential TFs by isolating and identify those TFs. We employ Scot Wolfe's B1H system, which provides a robust method to characterize protein-DNA interactions, as a complementary approach to clone and identify the TFs that bind each SNP region (FIG. 18). This system has been designed to detect binding sites for known TFs. We modify this system to clone unknown TFs rather than unknown binding sites. We identify TFs that bind the promoter regions of NAMPT by cloning 28mer binding sites for each of the four SNPs into the pH3U3 vector. cDNA libraries isolated from SW982 cells cultured under normoxic and hypoxic conditions, and/or cytokine stimulated, are inserted in frame into pB1H1/2 expression vectors. The pH3U3 binding site vectors are used to select cDNA clones that interact with the 28mer site. Positively selected clones are sequenced to identify TF and validated by EMSA and co-immunoprecipitation assays, where their cognate antibodies are available. We include THP-1, and HL-60 cells in these studies where warranted.

### Example XXI

### Determination of the Regulation of NAMPT in the Context of Multiple Tissues and Cell Types Upon Induction of Arthritis

This example is designed to test the hypothesis that a humanized NAMPT promoter mouse model will allow a systems approach to elucidate the regulation of NAMPT in the context of multiple tissues and cell types upon induction of arthritis. We interchange the mouse promoter with selected human promoters to generate humanized NAMPT promoter mice. We use a service provider (Creative Animodel, Shirley, N.Y.) since the UMKC-LARC does not possess transgenic mouse facilities. We utilize these new mouse models to study CIA as previously described.

### Statistical Analyses.

Parametric statistical analyses are performed using Sigmaplot analysis software as described in EXAMPLE X.

### Results

We characterize the ability of the promoter SNPs, and their haplotypes, to alter NAMPT expression and to disrupt TF binding. We isolate at least four TFs that bind to the corresponding SNP binding sites. In addition, we developed humanized mice possessing selected haplotypes of the human NAMPT promoter. These experiments substantiate our hypothesis that each SNP exerts differential allelic effects on NAMPT expression by altering its binding affinity of potential transcription factors, which underlie their functional association with arthritis. We further identify and characterize TFs that regulate NAMPT expression.

### Example XXII

### Determination of NAMPT Mediation of TNF-α Induced Inhibition of SP-B

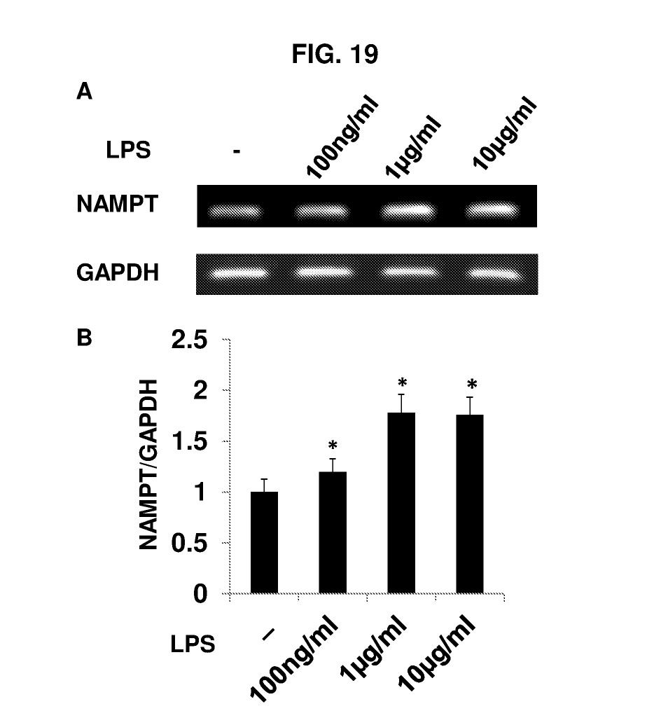

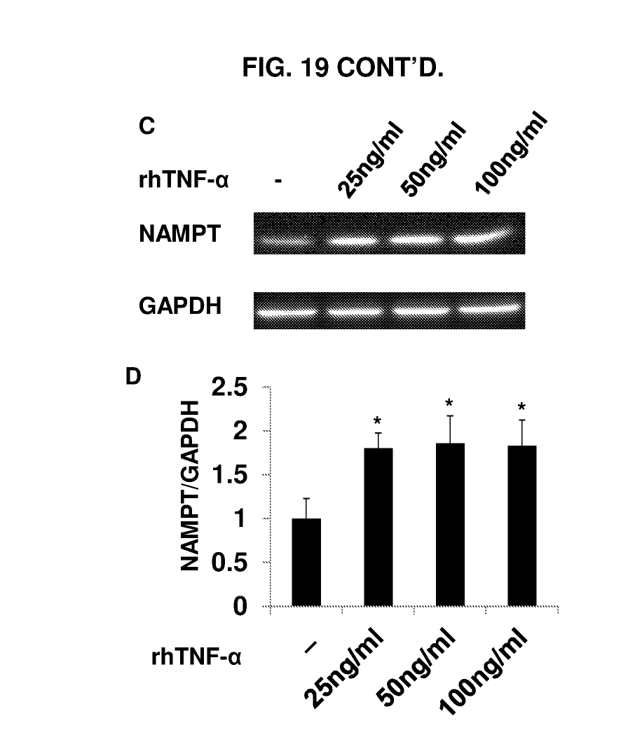

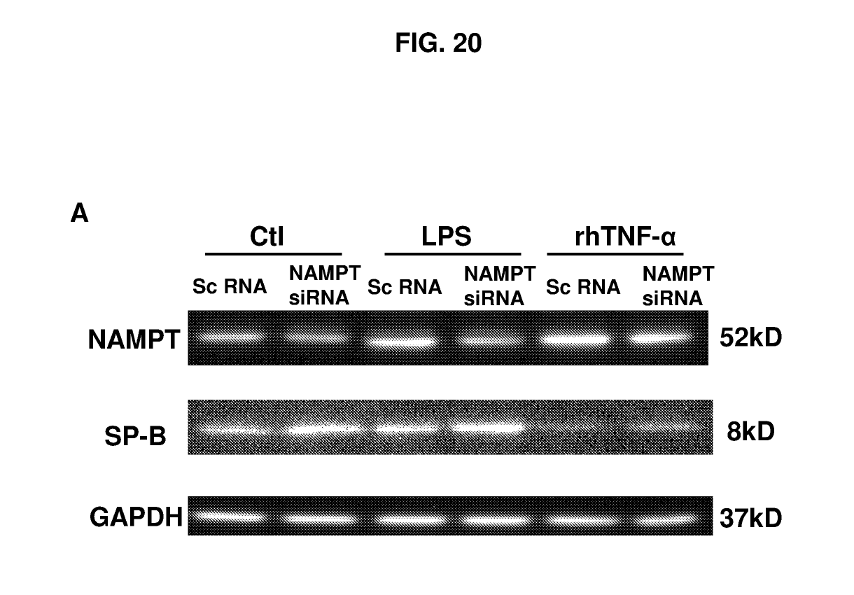

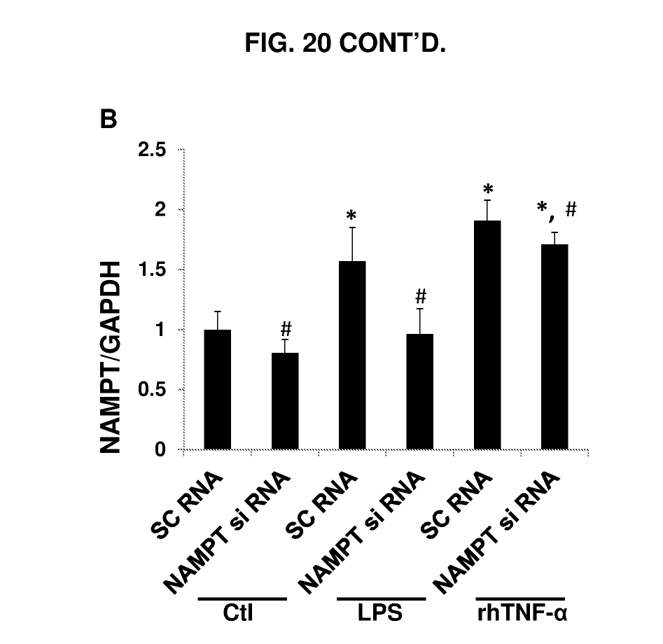

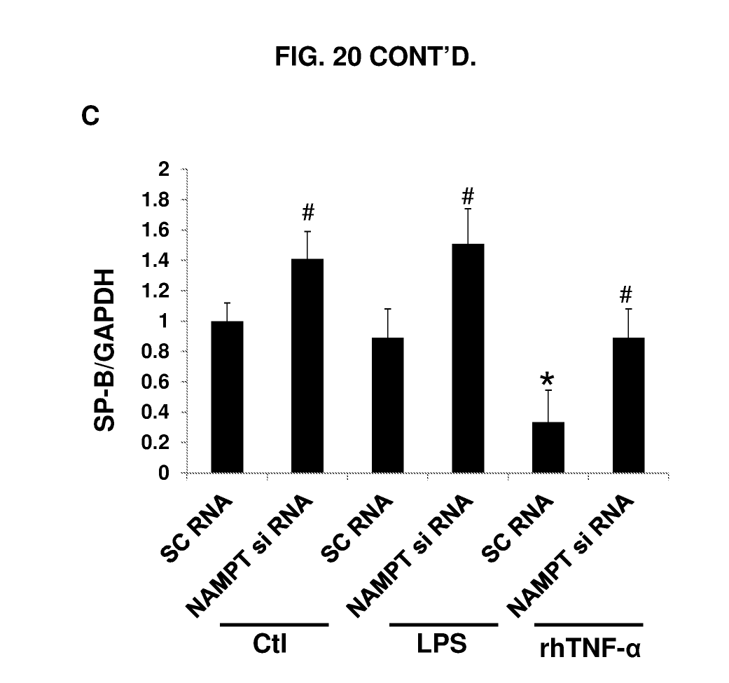

This example determines that NAMPT mediates TNF-α induced inhibition of SP-B in H441 cells and A549 cells. In order to confirm the role of NAMPT in H441 cells, the cells were first treated with different doses of either LPS or TNF-α. The results demonstrated that LPS (FIGS. 19 A and 19B) or TNF-α (FIGS. 19 C and 19D) treatment for 24 hours significantly increased NAMPT protein expression levels in a dose-dependent manner in H441 cells. Such results suggest that NAMPT is involved in inflammatory processes in both clara cells and alveolar type II cells, which are the source of secreting SP-B. Because a previous study reported that TNF-α inhibited expression of SP-B, the involvement of NAMPT in this process was investigated. NAMPT was knocked down with siRNA for 48 hours and then treated with/without either LPS or TNF-α in H441 cells. To transfect NAMPT stealth siRNA into human A549 cells and H441 cells, cells were seeded overnight in the regular growth medium (without antibiotics) so that they would be 80-90% confluent at the time of transfection. For each transfection in 48-well plates, 25 pmol NAMPT stealth siRNA or scramble RNA was diluted in 12.5 μl OptiMEM I without serum and gently mixed with 0.5 μl Lipofectamine 2000 diluted in the 12.5 μl Opti-MEM I (Cat. No. 31985-062, Invitrogen, Carlsbad, Calif., USA). After being incubated for 15 minutes at room temperature, NAMPT stealth siRNA & Lipofectamine 2000 complexes or scramble RNA & Lipofectamine 2000 complexes were added to each well. Cell culture plates were gently mixed by rocking back and forth. The amount of PBEF stealth siRNA and Lipofectamine 2000 were adjusted according to the different sizes of cell culture plates. Transfected cells were further incubated at 37° C. for 48 hours until the treatment with LPS or TNF-α was performed. The results (FIG. 20) showed that TNF-α significantly decreased SP-B expression, whereas LPS didn't. Down regulation of NAMPT significantly increased SP-B expression and rescued TNF-α induced inhibition of SP-B expression in H441 cells. A similar effect that NAMPT silencing rescued TNF-α induced inhibition of SP-B was observed in A549 cells. Thus, after silencing NAMPT expression, SP-B expression was significantly increased over baseline, indicating NAMPT's effect on inhibiting SP-B expression. TNF-α significantly decreased SP-B expression, while silencing of NAMPT rescued the TNF-α induced inhibition of SP-B. However, LPS stimulation didn't exhibit the same inhibition of SP-B with TNF-α, but silencing of NAMPT still significantly increased SP-B expression. These results demonstrate that NAMPT is one of the inhibited factors of SP-B, that TNF-α induced SP-B inhibition is partly NAMPT-dependent, and NAMPT mediates TNF-α induced inhibition of SP-B in both of the epithelial cells.

### Example XXIII

### Determination of Nonenzymatic and Enzymatic Roles in the Inhibition of SP-B Expression by NAMPT

This example determined that NAMPT inhibits SP-B expression mainly via its nonenzymatic activity and partly via its enzymatic activity. NAMPT is a multiple function protein that is not only involved in the mammalian salvage pathway of NAD synthesis via its enzymatic activity, but is also involved in the regulation of inflammatory cytokine expression in pulmonary epithelial cells via its nonenzymatic and AP-1 dependent mechanism. To examine whether NAMPT regulates SP-B expression via its NAMPT activity, H441 cells were transfected with either wild-type pCAGGS-hPBEF or pCAGGS-hPBEF (H247E, HE) which are the human NAMPT mutants that have very low NAMPT activities. The results (FIG. 21 A) showed that overexpression of mutant NAMPT similarly and significantly inhibited the SP-B expression protein levels as with wild-type NAMPT. The controls, pCAGGS vector only, had no effect. On the other hand, H441 cells pretreated with FK866, which is the intracellular NAMPT enzymatic inhibitor, stimulated the cells with or without TNF-α. The results (FIG. 21 B) showed that SP-B expression increased after treatment with FK866, however treatment with FK866 did not significantly rescue TNF-α induced inhibition of SP-B. Consideration of the results that knockdown of NAMPT with siRNA could rescue TNF-α induced inhibition of SP-B, while inhibiting NAMPT enzymatic activity with FK866 did not have that effect, and overexpression of mutant NAMPT has the similar phenomenon of inhibiting SP-B expression with wild-type NAMPT, NAMPT inhibits SP-B expression via both of nonenzymatic and enzymatic activity at baseline. However, the nonenzymatic activity of NAMPT plays a more important role in regulating TNF-α induced SP-B inhibition.

### Example XXIV

### Determination of the Involvement of the JNK Pathway in NAMPT-Inhibition of SP-B in H441 Cells

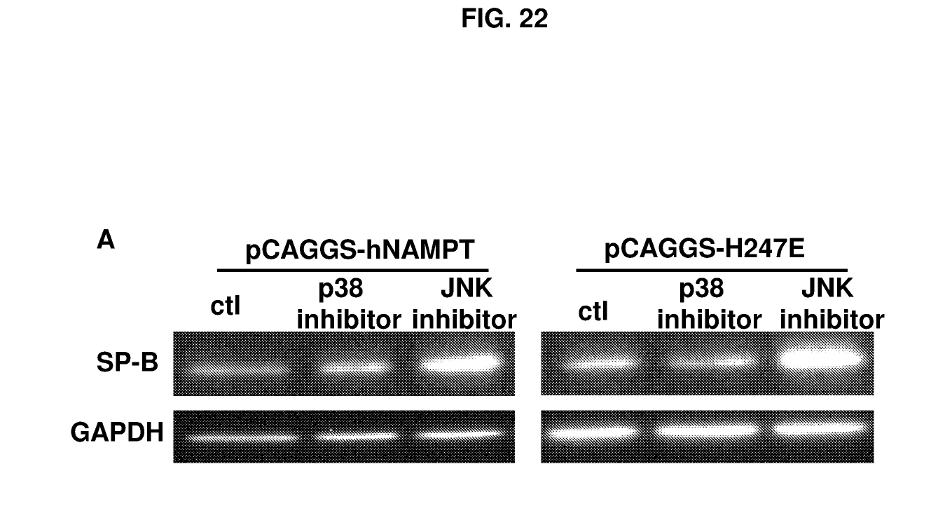

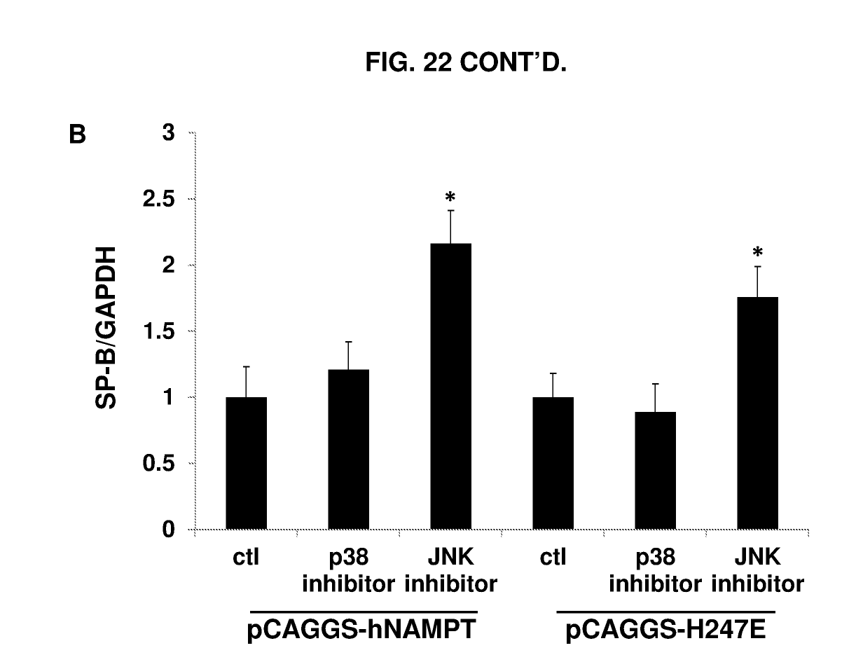

This example demonstrates that the JNK pathway is involved in the NAMPT-inhibition of SP-B in H441 cells. NAMPT increases AP-1 binding to the IL-8 promoter to activate transcription in epithelial cells via the p38 MAPK pathway and the JNK pathway. Activated AP-1 inhibits SP-B expression. To determine the relationship, H441 cells were pretreated in the absence or presence of either a p38 pathway inhibitor, SB203580, or a JNK pathway inhibitor, SP600125, for 6 hours, then transfected with either wild-type pCAGGS-hPBEF or mutant type pCAGGS-H247E. The results (FIG. 22) showed that the JNK inhibitor significantly attenuated the NAMPT induced inhibition of SP-B, although the p38 inhibition was not. These data suggested that wild-type PBEF- or mutant PBEF-mediated inhibition of SP-B expression is due in part via the JNK pathway.

### Example XXV

### Cell Specific Knockdown of NAMPT in Epithelial Cells Exhibits Attenuated Acute Lung Injury in Mice

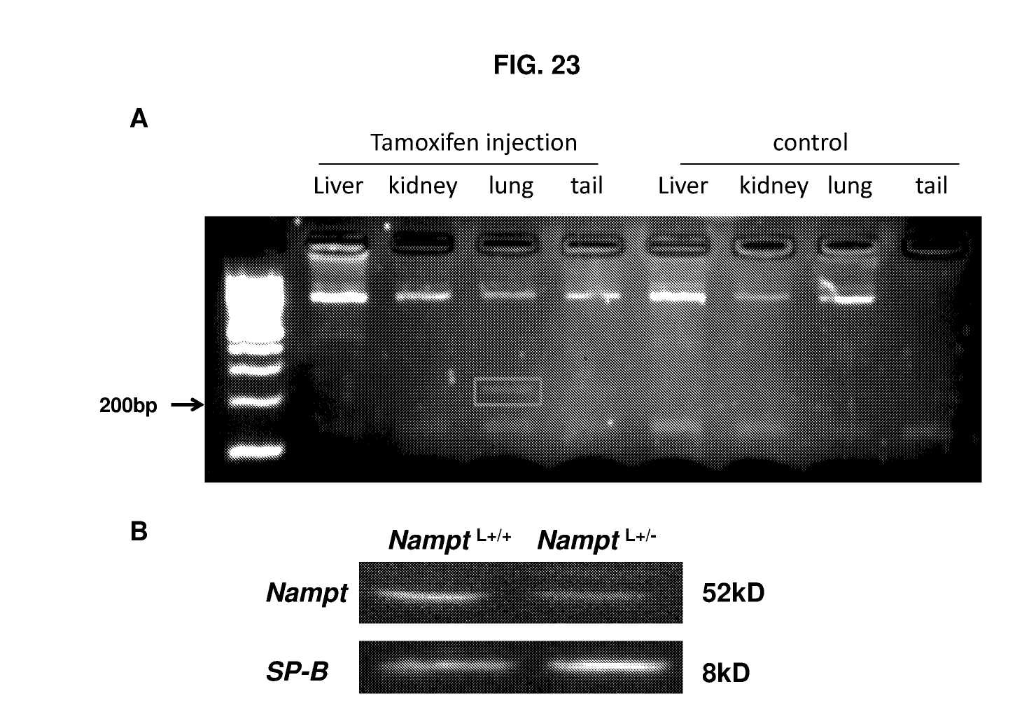

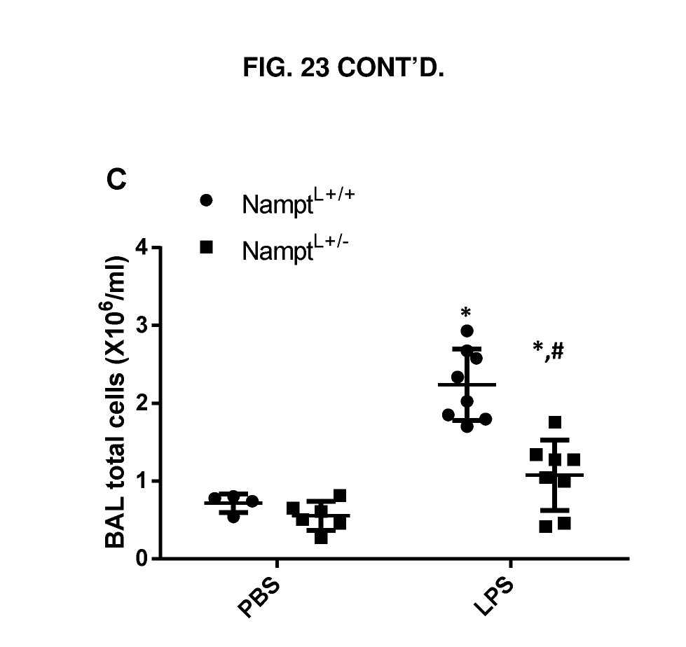

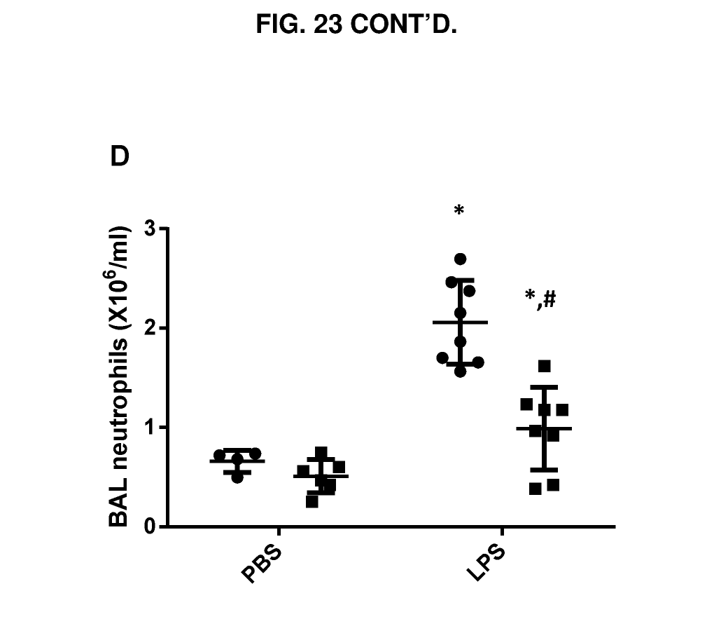

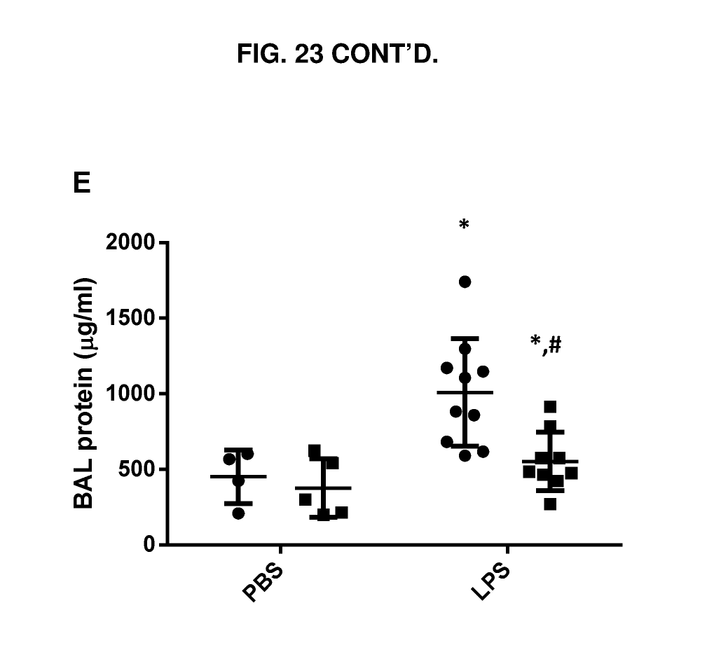

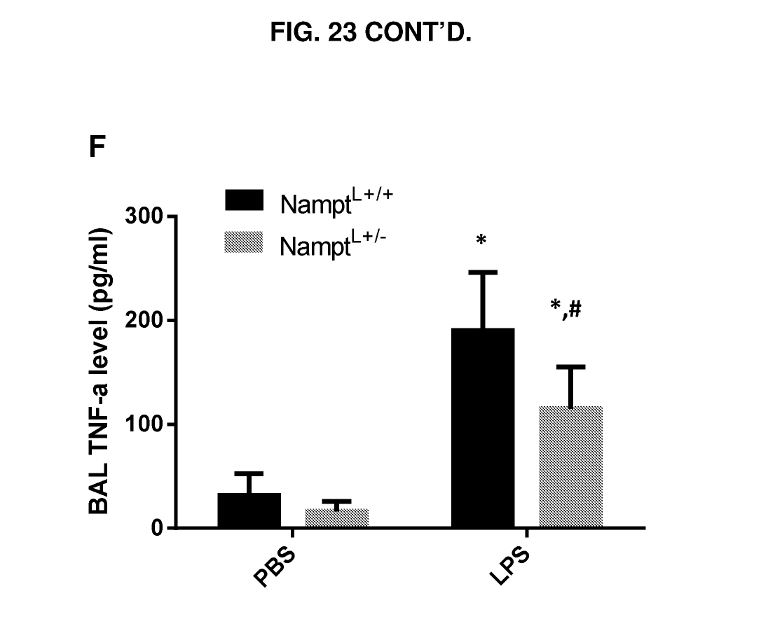

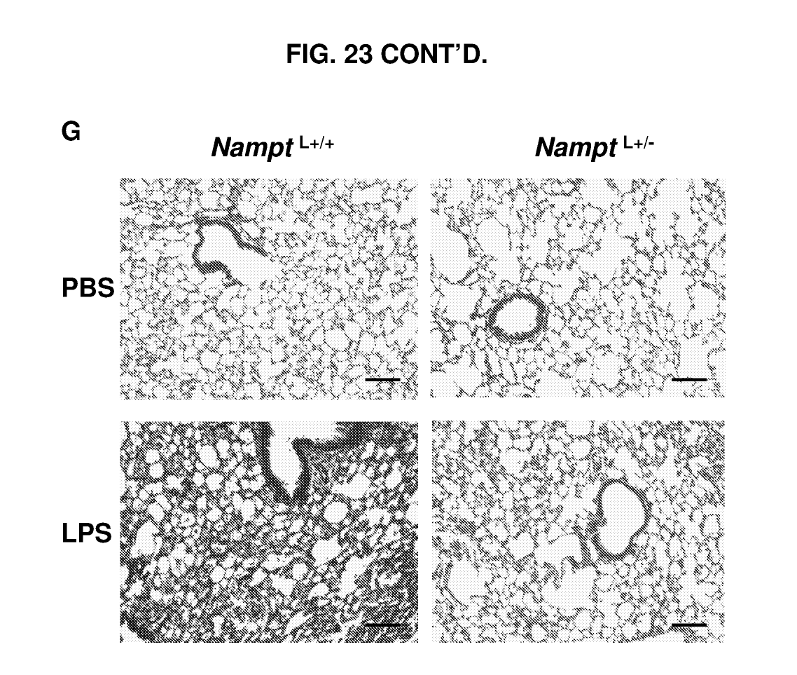

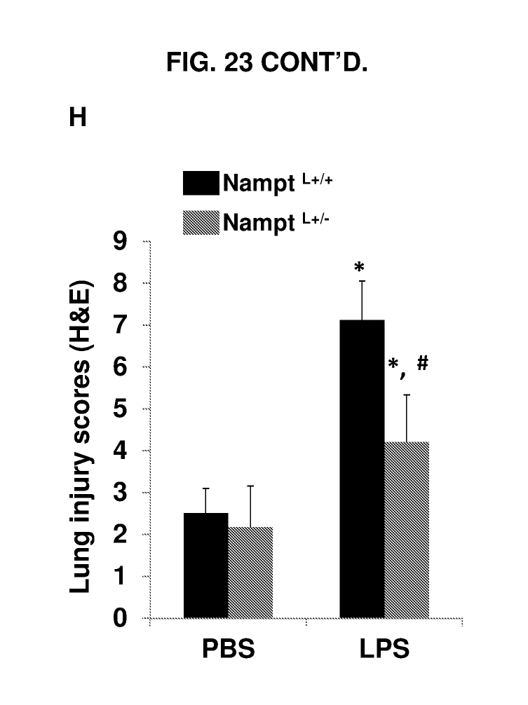

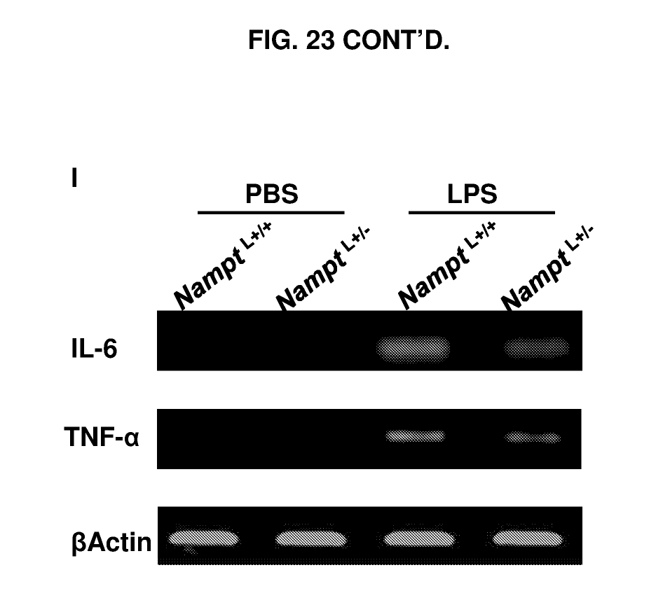

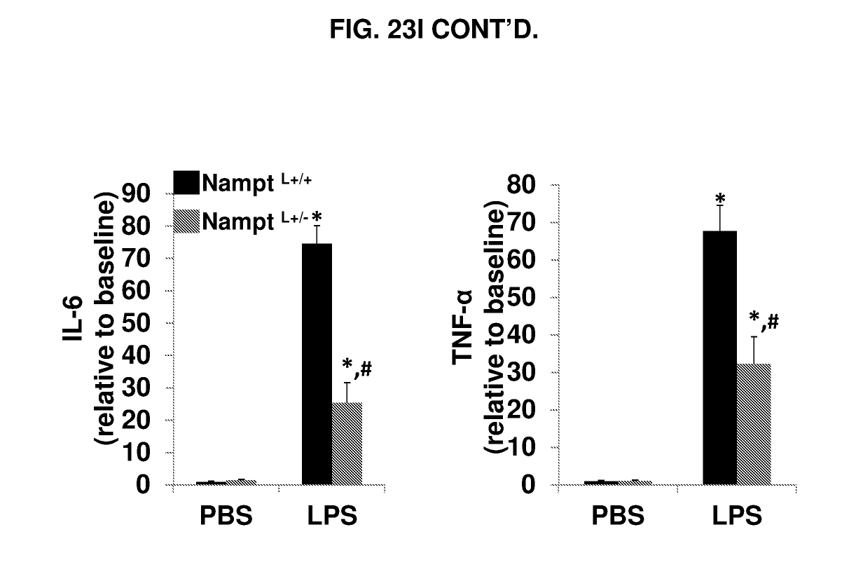

This example demonstrates that cell specific knockdown of NAMPT in epithelial cells exhibited attenuated acute lung injury in mice. Knockdown of NAMPT increases SP-B expression and rescues the TNF-α induced inhibition of SP-B in vitro. A heterozygous NAMPT L+/− mouse line with a targeted deletion of a single NAMPT allele in epithelial cells was generated in order to examine the NAMPT function in vivo. NAMPT L+/− mice were injected with tamoxifen prior to the experiment to induce NAMPT knockdown in epithelial cells. Lung specific NAMPT gene deletion was confirmed by PCR of genotyping of mouse lung tissue and organs from NAMPT L+/− mice (FIG. 23 A). Western blot analysis of NAMPT expression demonstrated lower protein levels of NAMPT and higher protein levels of SP-B in lung epithelial cells from NAMPT L+/− mice than in those from NAMPT L+/+ mice (FIG. 23 B). Western blot analysis was performed as described previously. Briefly, cell lysates were mixed with RIPA buffer and boiled for 5 min. Equal amounts of proteins (for NAMPT determination was 10 μg, for SP-B determination was 30 μg) were resolved by NaDodSO4-PAGE and transferred onto polyvinylidene fluoride membranes (Immobilon P; Millipore). The membranes were probed with specific Abs, followed by detection with HRP-conjugated goat anti-rabbit IgG or anti-mouse IgG. Bands were visualized by ECL (Pierce ECL Western Blotting Substrate Cat#32106; ThermoFisher, Rockford, Ill.) and quantified by AlphaView software. LPS was utilized to induce ALI model in wild type (NAMPT L+/+) and NAMPT L+/− mice. The results showed that total BAL cell counts (FIG. 23 C), total BAL neutrophil count (FIG. 23 D), BAL protein concentration (FIG. 23 E), and TNF-α levels in the BAL supernatant (FIG. 23 F) were significantly lower from NAMPT L+/− mice compared to those from NAMPT L+/+ mice in the LPS induced lung injury models. These results were also consistent with histologic evaluation (FIG. 23 G) that neutrophilic infiltration, interstitial edema and alveolar wall damage were ameliorative in sections of lungs from NAMPT L+/− mice when compared to those from NAMPT L+/+ mice. Additionally, total lung injury scores (FIG. 23 H) from NAMPT L+/− mice were significantly lower than those from NAMPT L+/+ mice in the LPS induced lung injury group.

### Example XXVI

### The Ad-SPC-NAMPT Antibody Gene has a Therapeutic Effect on ALI

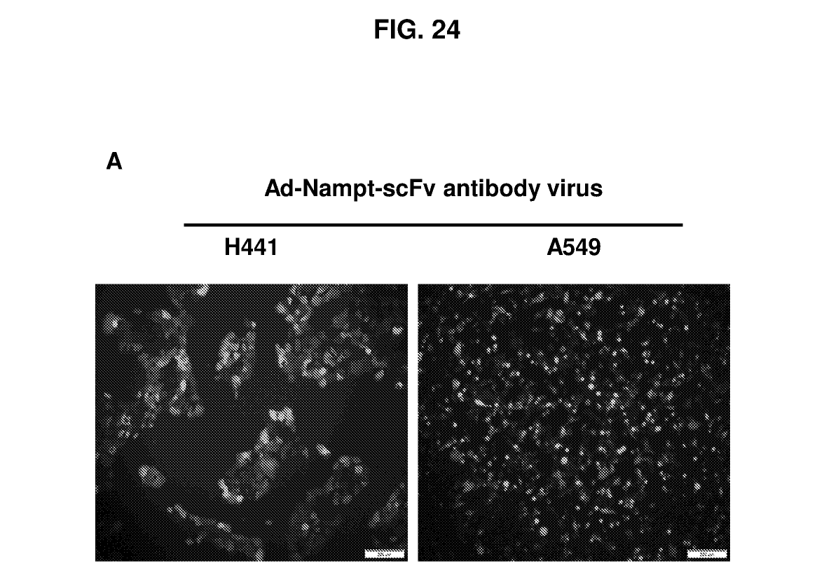

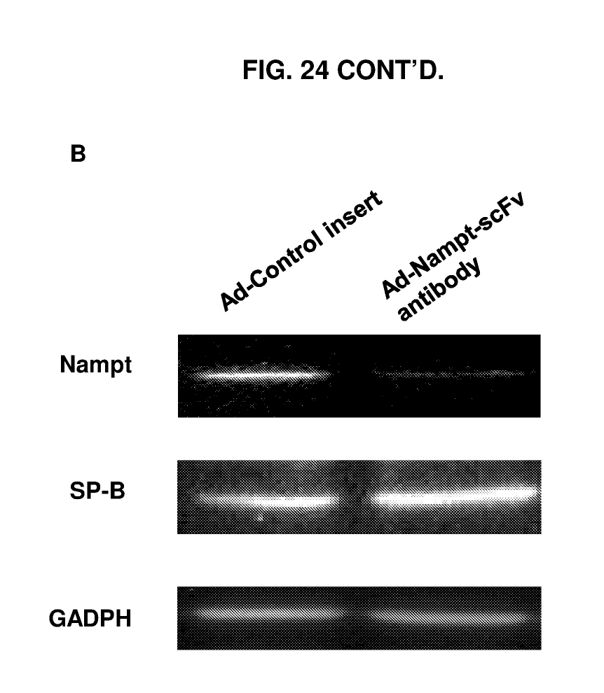

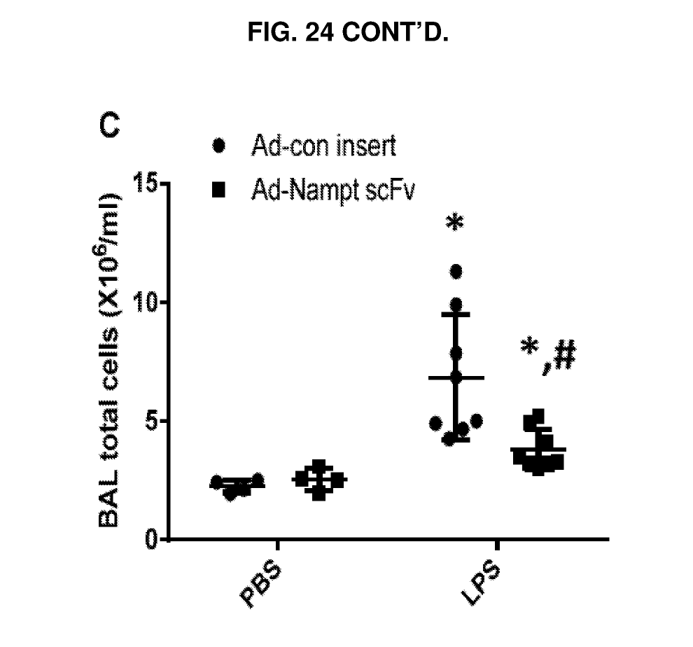

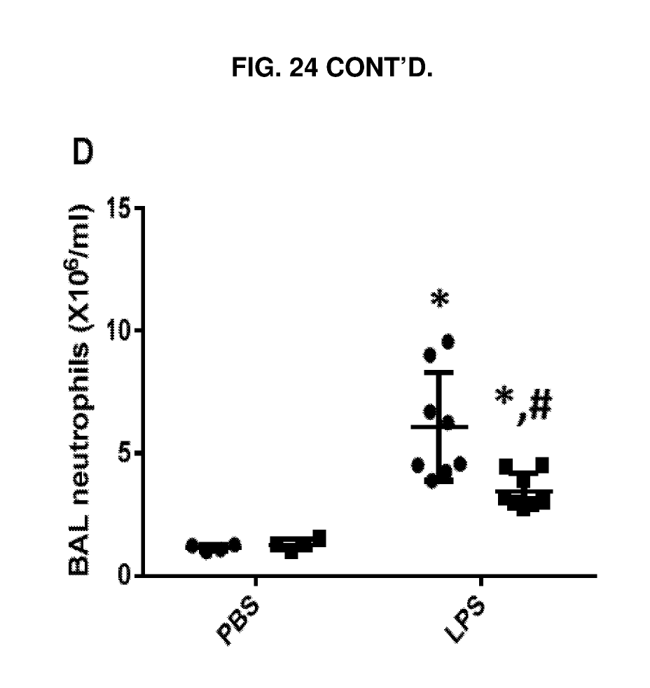

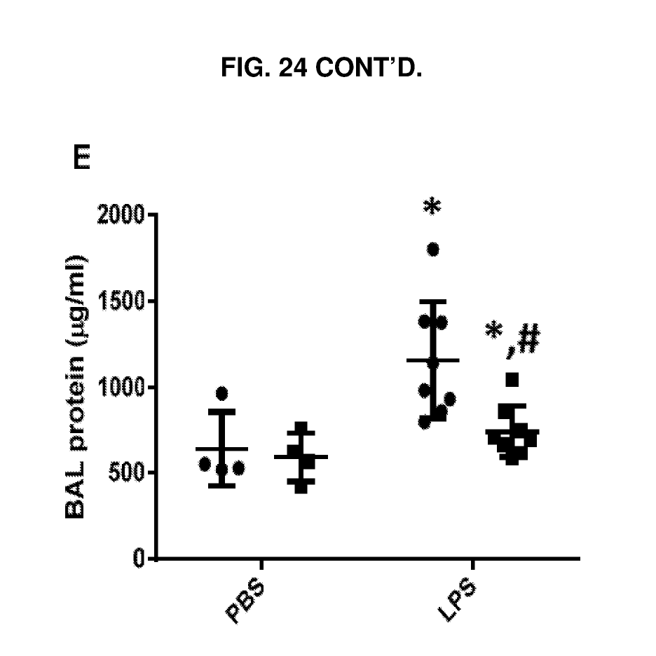

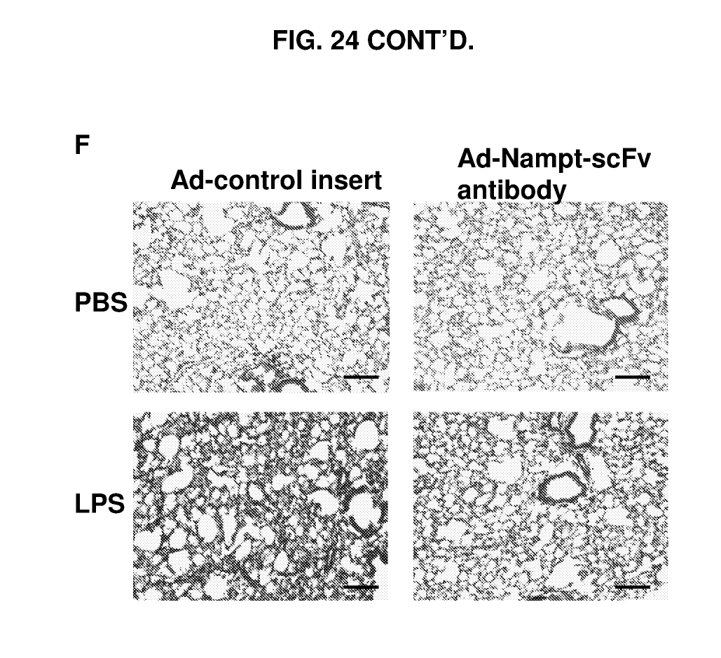

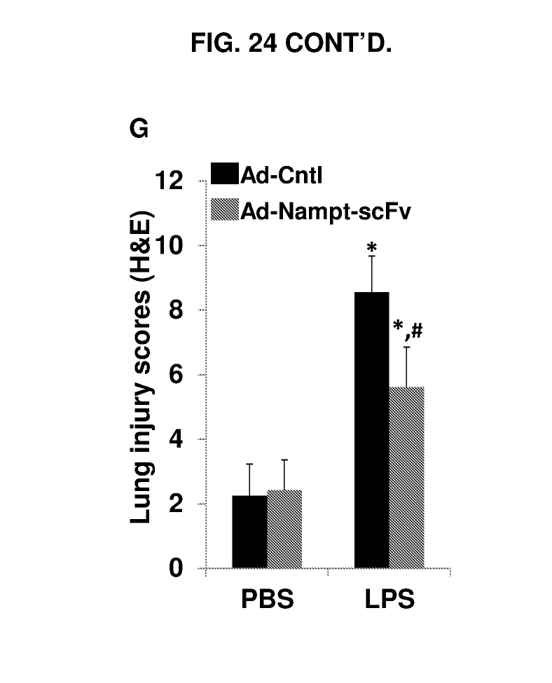

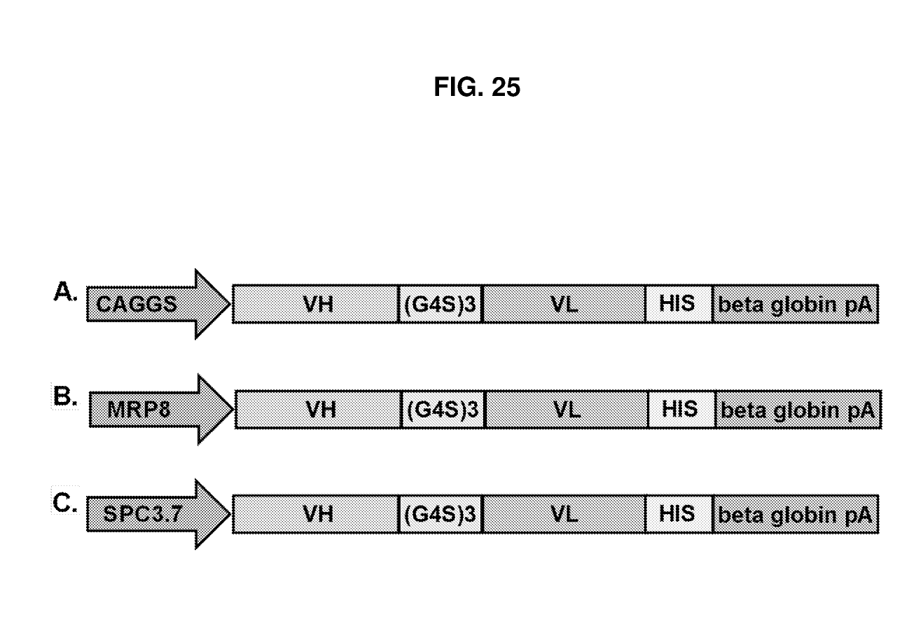

This example demonstrates that the Ad-SPC-NAMPT antibody gene has a therapeutic effect on ALI. The findings support that lung epithelial cell specific NAMPT knockdown attenuated lung injury, which demonstrated a need for the development of a lung epithelial cell specific NAMPT knockdown as a new therapeutic modality in ALI. An adenovirus expressing a NAMPT scFV antibody (Ad-SPC-NAMPT-scFv) driven by the lung epithelium specific human SPC promoter utilizing the Adeno-X™ Adenoviral System 3 was generated. An in vitro tissue culture model using H441 cells to test the anti-nampt potential of our Ad-SPC-NAMPT-scFv viral was established. Fluorescence images of H441 cells and A549 cells infected with Ad-SPC-NAMPT-scFv viral indicated the high efficiency of adenovirus infection in epithelial cells. Western blot showed that NAMPT expression was decreased, while SP-B expression was increased in Ad-SPC-NAMPT-scFv treated H441 cells. In vivo assays were then performed using Ad-SPC-NAMPT-scFv (1×109 IFU) or the appropriate controls Ad-control insert (1×109 IFU). Ad constructs were injected into individual wild type mice intratracheally, 3 days later the adenovirus treated mice were challenged with LPS or PBS. The mice were then sacrificed for tissue isolation to determine the therapeutic effect of the viral constructs. The results showed that BAL protein concentration, total BAL cell counts, total BAL neutrophils, lung tissue histology and lung injury scores from Ad-SPC NAMPT antibody (FIG. 24) treated mice were significantly lower than those from control virus injected mice, indicating that anti-NAMPT therapy in epithelial cells attenuates the LPS-induced damage in C57BL/6 wild type mice.

Reagents used in these examples include: RPMI 1640 (Cat#: 11875), DMEM (Cat#: 11965), FBS (Cat#: 14190), and penicillin-streptomycin (Cat#: 15140) were purchased from ThermoFisher, life technologies, NY. Escherichia coli 0111:B4 endotoxin (LPS, Cat# L4391), p38 inhibitor SB239063, JNK inhibitor SP600125 was purchased from Sigma-Aldrich (St. Louis, Mo.). TNF-α ELISA kit (Cat# MTA00B) was obtained from R&D system (Minneapolis, Minn.). NAMPT antibody was purchased, SP-B antibody was obtained from Santa Cruz, FK866.

Cell cultures used in these examples include: A549 cell (Cat. No. CCL-185™), H441 cell (Cat. No. HTB-174™) and HL-60 cell (Cat. No. CCL-240™), were obtained from ATCC (Manassas, Va., USA). A549 cells were maintained in DMEM supplemented with 10% fetal bovine serum, 2 mM glutamine, penicillin/streptomycin. H441 cells and HL-60 cells were maintained in RPMI supplemented with 10% fetal bovine serum, 2 mM glutamine, penicillin/streptomycin. All cells were cultured at 37° C. in a humidified atmosphere of 5% CO2, 95% air. Cells from each primary flask were detached with 0.25% trypsin, resuspended in fresh culture media, and seeded into 6-well plates for Western blotting or RT-PCR analysis, or seeded into the 48-well plates for ELISA or HL-60 adhesion assay.

Isolation of RNA and RT-PCR analysis. Total RNA was isolated from A549 cells with TRIZOL solution (Cat. No. 15596-018, Invitrogen, Carlsbad, Calif., USA) according to the supplier's instructions. RT-PCR was performed using Invitrogen RNA PCR kit (Superscript III, 18080-044). PCR products were separated on a 1.5% agarose gel and stained by Ethidium Bromide (0.5 μg/ml). The band image was acquired using an Alpha Imager and analyzed by the AlphaEase™ Stand Alone Software (Alpha Innotech Corp., San leandro, CA, USA).

LPS-induced ALI animal mode. Mice were anesthetized with PS (100 mg/kg and 5 mg/kg ip), intubated with a 20-g catheter, and administered intratracheally PBS or LPS (2 mg/kg per mice, diluted in phosphate-buffered saline (PBS), Sigma, St. Louis Mo.), after recovery, the mice returned to their cages. After 24 hours, the mice were anesthetized and intubated again.

Construction of recombinant adenovirus vectors. The plasmid pAdX-PRLS-ZsGreen1 was constructed previously. The SPC-NAMPT-scFv antibody, and their control SPC-NAMPT-reverse scFv antibody cassette were inserted into pAdX-PRLS-ZsGreen1 individually. The plasmid was then transfected into the Adeno-X 293 Cell Line. The recombinant adenoviruses were isolated and purified by Maxi purification kit. The viral titers were determined by Adeno-X™ Rapid Titer Kit.

In vivo adenovirus transduction. 8-10 week mice were anesthetized with PS, intubated with a 20-g catheter and administered intratracheally with 100 μl of either Ad-SPC-NAMPT-scFv or Ad-control-insert virus solution (1×109 ifu). After recovery, the mice returned to their cages. 72 hours later the mice were anesthetized and intubated for challenge with either PBS or LPS. 24 hours later, the mice were sacrificed.

Statistics. Statistical analyses were performed using the Sigma Stat (ver 13.0, Systat Software, Inc., San Jose, Calif.). Results are expressed as means±S.D. of more than three samples for each group from at least two independent experiments. Two group comparisons were done by unpaired t-test. Three or more group comparisons were carried out using ANOVA followed by a Holm-Sidak test, p<0.05 was considered statistically significant.

1. A method of inhibition of nicotinamide phosphoribosyltransferase (NAMPT) in a targeted cell, said method comprising:
administering to a human or animal a cDNA sequence encoding a recombinant single-chain variable fragment antibody that recognizes and binds to NAMPT comprising:
a variable region of heavy (V_{H}) chain of immunoglobulin and a variable region of light (V_{L}) chain of immunoglobulin; and
a peptide linker disposed between and connecting the and V_{H} variable regions,
wherein said antibody is specifically expressed and inhibits said NAMPT in a targeted cell of said human or animal.

2.-11. (canceled)

12. The method of claim 1, wherein said single-chain variable fragment antibody comprises an amino acid sequence having at least 96% sequence homology with SEQ ID NO. 2 or SEQ ID NO. 4.

13. The method of claim 1, wherein said single-chain variable fragment antibody comprises six portions, wherein the first portion is an amino acid sequence having at least 96% sequence homology with SEQ ID NO. 10, the second portion is an amino acid sequence having at least 96% sequence homology with SEQ ID NO. 12 or SEQ ID NO. 14, the third portion is an amino acid sequence having at least 96% sequence homology with SEQ ID NO. 16 or SEQ ID NO. 18, the fourth portion is an amino acid sequence having at least 96% sequence homology with SEQ ID NO. 20, the fifth portion is an amino acid sequence having at least 96% sequence homology with SEQ ID NO. 22 or SEQ ID NO. 24, and the sixth portion is an amino acid sequence having at least 96% sequence homology with a sequence SEQ ID NO. 26 or SEQ ID NO. 28.

14. (canceled)

15. The method of claim 1, wherein the peptide linker is selected from the group consisting of the HIV1 p24 linker and the (G4S)3 linker.

16. The method of claim 1, wherein the cDNA sequence includes a promoter.

17. The method of claim 16, wherein the promoter is specific for said targeted cell wherein the antibody will be expressed.

18. The method of claim 17, wherein the targeted cell is selected from the group consisting of neutrophils, fibroblasts, macrophages, and epithelial cells.

19. The method of claim 1, wherein said administering comprises administering an adenovirus having a foreign insert therein, wherein the foreign insert comprises said cDNA sequence.

20.-22. (canceled)

23. The method of claim 19, said foreign insert further comprising a specific promoter for the targeted cell, wherein the specific promoter is selected from the group consisting of the SPC or MRP8 promoters.

24.-29. (canceled)

30. A host cell transformed with a sequence encoding at least one anti-NAMPT antibody, wherein said anti-NAMPT antibody is a recombinant single-chain variable fragment antibody comprising a fusion protein of a variable region of heavy (V_{H}) chain of immunoglobulin and a variable region of light (V_{L}) chain of immunoglobulin connected with a peptide linker.

31. The host cell of claim 30, wherein said host cell is selected from the group consisting of epithelial cells, neutrophils, fibroblasts, and macrophages.

32.-35. (canceled)

36. The host cell of claim 30, wherein the anti-NAMPT antibody comprises an amino acid sequence having at least 96% sequence homology with SEQ ID NO. 2 or SEQ ID NO. 4.

37.-40. (canceled)

41. The anti-NAMPT cDNA clone of claim 88, said cDNA having a sequence at least 96% homologous to nucleotide sequence SEQ ID No. 1 or SEQ ID No. 3.

42. The anti-NAMPT cDNA clone according to claim 88, encoding a sequence at least 96% homologous to amino acid sequence SEQ ID No. 2 or SEQ ID No. 4.

43.-69. (canceled)

70. The method of claim 1, further comprising treating LPS-induced lung injury in a subject in need thereof by administration of the cDNA to the subject.

71.-84. (canceled)

85. The method of claim 1, wherein said antibody is administered as a dimer, timer, or tetramer of said single-chain variable fragment.

86. The method of claim 85, wherein said antibody is a dimer comprising an amino acid sequence identified as SEQ ID NO. 6.

87. The method of claim 1, wherein said administering comprises injection, oral introduction, or sublingual introduction of said cDNA sequence into said human or animal.

88. An anti-NAMPT cDNA clone encoding a cell-specific anti-NAMPT antibody, wherein said anti-NAMPT antibody is a recombinant single-chain variable fragment antibody comprising a fusion protein of a variable region of heavy (V_{H}) chain of immunoglobulin and a variable region of light (V_{L}) chain of immunoglobulin connected with a peptide linker.

89. The anti-NAMPT cDNA clone of claim 88, wherein said clone comprises:
a complementary determining region 2 selected from the group consisting of SEQ ID No. 12 and SEQ ID No. 14;
a complementary determining region 3 selected from the group consisting of SEQ ID No. 16 and SEQ ID No. 18;
a complementary determining region 5 selected from the group consisting of SEQ ID No. 22 and SEQ ID No. 24; and/or
a complementary determining region 6 selected from the group consisting of SEQ ID No. 26 and SEQ ID No. 28.

90. An adenovirus having a foreign insert therein, wherein the foreign insert comprises an anti-NAMPT cDNA clone according to claim 88.


SEQUENCE LISTING (40 sequences)

SEQ ID NO: 1 - DNA, 762 bp, Homo sapiens
  misc_feature (1)..(6): EcoRI sequence
  misc_feature (13)..(15): Start Codon
  misc_feature (106)..(120): CDR1
  misc_feature (163)..(222): CDR2 scFv1
  misc_feature (310)..(330): CDR3 scFv1
  misc_feature (364)..(408): 15-mer linker sequence
  misc_feature (481)..(513): CDR4
  misc_feature (559)..(579): CDR5 scFv1
  misc_feature (682)..(702): CDR6 scFv1
  misc_feature (736)..(753): Histidine Tag
  misc_feature (754)..(756): Stop Codon
  misc_feature (757)..(762): Kpnl
gaattcgccaccatggaggtgcagctgttggagtctgggggaggcttggtacagcctggggggtccctgagactctcctgtgcagcctctggattcacctttagcagctatgccatgagctgggtccgccaggctccagggaaggggctggagtgggtctcagtgattaatgttcctggtaatcgtacatcgtacgcagactccgtgaagggccggttcaccatctccagagacaattccaagaacacgctgtatctgcaaatgaacagcctgagagccgaggacacggccgtatattactgtgcgaaaaagacgcgtcggtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccgttttgcaaagtggggtcccatcaaggttcagtggcagtggatctgggacagatttcactctcaccatcagcagtctgcaacctgaagattttgcaacttactactgtcaacagcgtgttcgttctcctgctacgttcggccaagggaccaaggtggaaatcaaacggcatcatcaccatcaccattaaggtacc

SEQ ID NO: 2 - protein, 247 aa, Homo sapiens
  MISC_FEATURE (32)..(36): CDR1
  MISC_FEATURE (51)..(70): CDR2 scFv1
  MISC_FEATURE (99)..(105): CDR3 scFv1
  MISC_FEATURE (157)..(167): CDR4
  MISC_FEATURE (183)..(189): CDR5 scFv1
  MISC_FEATURE (224)..(230): CDR6 scFv1
  MISC_FEATURE (242)..(247): Histidine Tag
MEVQLLESGGGLVQPGGSLRLSCAASGFTFSSYAMSWVRQAPGKGLEWVSVINVPGNRTSYADSVKGRFTISRDNSKNTLYLQMNSLRAEDTAVYYCAKKTRRFDYWGQGTLVTVSSGGGGSGGGGSGGGGSTDIQMTQSPSSLSASVGDRVTITCRASQSISSYLNWYQQKPGKAPKLLIYGASVLQSGVPSRFSGSGSGTDFTLTISSLQPEDFATYYCQQRVRSPATFGQGTKVEIKRHHHHHH

SEQ ID NO: 3 - DNA, 768 bp, Homo sapiens
  misc_feature (1)..(6): EcoRI sequence
  misc_feature (13)..(15): Start Codon
  misc_feature (106)..(120): CDR1
  misc_feature (163)..(222): CDR2 scFv2
  misc_feature (310)..(330): CDR3 scFv2
  misc_feature (364)..(408): 15-mer linker sequence
  misc_feature (481)..(513): CDR4
  misc_feature (559)..(582): CDR5 scFv2
  misc_feature (682)..(702): CDR6 scFv2
  misc_feature (742)..(759): Histidine Tag
  misc_feature (760)..(762): Stop Codon
  misc_feature (763)..(768): Kpnl
gaattcgccaccatggaggtgcagctgttggagtctgggggaggcttggtacagcctggggggtccctgagactctcctgtgcagcctctggattcgcctttagcagctatgccatgagctgggtccgccaggctccagggaaggggctggagtgggtctcaaatatttctgggcggggtgctaagacaacgtacgcagactccgtgaagggccggttcaccatctccagagacaattccaagaacacgctgtatctgcaaatgaacagcctgagagccgaggacacggccgtatattactgtgcgaaaacgtatcttgtgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatcgtgcatcctctttgcaaagtggggtcccatcaaggttcagtggcagtggatctgggacagatttcactctcaccatcagcagtctgcaacctgaagattttgcaacttactactgtcaacagatttcggctacgcctactacgttcggccaagggaccaaggtggaaatcaaacgggcggcccatcatcaccatcaccattaaggtacc

SEQ ID NO: 4 - protein, 249 aa, Homo sapiens
  MISC_FEATURE (32)..(36): CDR1
  MISC_FEATURE (41)..(60): CDR2 scFv2
  MISC_FEATURE (90)..(96): CDR3 scFv3
  MISC_FEATURE (108)..(122): 15-mer linker sequence
  MISC_FEATURE (147)..(157): CDR4
  MISC_FEATURE (173)..(180): CDR5 scFv2
  MISC_FEATURE (214)..(220): CDR6 scFv2
  MISC_FEATURE (234)..(239): Histidine Tag
MEVQLLESGGGLVQPGGSLRLSCAASGFAFSSYAMSWVRQAPGKGLEWVSNISGRGAKTTYADSVKGRFTISRDNSKNTLYLQMNSLRAEDTAVYYCAKTYLVFDYWGQGTLVTVSSGGGGSGGGGSGGGGSTDIQMTQSPSSLSASVGDRVTITCRASQSISSYLNWYQQKPGKAPKLLIYRASSLQSGVPSRFSGSGSGTDFTLTISSLQPEDFATYYCQQISATPTTFGQGTKVEIKRAAHHHHHH

SEQ ID NO: 5 - DNA, 1545 bp, Artificial Sequence
  Homo sapien combined with artificial ____
  misc_feature (1)..(6): EcoRI sequence
  misc_feature (13)..(15): Start Codon
  misc_feature (363)..(408): 15-mer linker sequence
  misc_feature (736)..(789): 18-mer linker sequence
  misc_feature (1141)..(1185): 15-mer linker sequence
  misc_feature (1519)..(1536): Histidine Tag
  misc_feature (1537)..(1539): Stop Codon
  misc_feature (1540)..(1545): EcoRI sequence
gaattcgccaccatggaggtgcagctgttggagtctgggggaggcttggtacagcctggggggtccctgagactctcctgtgcagcctctggattcacctttagcagctatgccatgagctgggtccgccaggctccagggaaggggctggagtgggtctcagtgattaatgttcctggtaatcgtacatcgtacgcagactccgtgaagggccggttcaccatctccagagacaattccaagaacacgctgtatctgcaaatgaacagcctgagagccgaggacacggccgtatattactgtgcgaaaaagacgcgtcggtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccgttttgcaaagtggggtcccatcaaggttcagtggcagtggatctgggacagatttcactctcaccatcagcagtctgcaacctgaagattttgcaacttactactgtcaacagcgtgttcgttctcctgctacgttcggccaagggaccaaggtggaaatcaaacggggtggttcctctagatcttcctcctctggtggcggtggctcgggcggtggtgggatggaggtgcagctgttggagtctgggggaggcttggtacagcctggggggtccctgagactctcctgtgcagcctctggattcgcctttagcagctatgccatgagctgggtccgccaggctccagggaaggggctggagtgggtctcaaatatttctgggcggggtgctaagacaacgtacgcagactccgtgaagggccggttcaccatctccagagacaattccaagaacacgctgtatctgcaaatgaacagcctgagagccgaggacacggccgtatattactgtgcgaaaacgtatcttgtgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatcgtgcatcctctttgcaaagtggggtcccatcaaggttcagtggcagtggatctgggacagatttcactctcaccatcagcagtctgcaacctgaagattttgcaacttactactgtcaacagatttcggctacgcctactacgttcggccaagggaccaaggtggaaatcaaacgggcggcccatcatcaccatcaccattaaggtacc

SEQ ID NO: 6 - protein, 508 aa, Artificial Sequence
  Homo sapien combined with _______
  MISC_FEATURE (118)..(132): 15-mer linker sequence
  MISC_FEATURE (242)..(259): 18-mer linker sequence
  MISC_FEATURE (377)..(391): 15-mer linker sequence
  MISC_FEATURE (502)..(508): Histidine Tag
MEVQLLESGGGLVQPGGSLRLSCAASGFTFSSYAMSWVRQAPGKGLEWVSVINVPGNRTSYADSVKGRFTISRDNSKNTLYLQMNSLRAEDTAVYYCAKKTRRFDYWGQGTLVTVSSGGGGSGGGGSGGGGSTDIQMTQSPSSLSASVGDRVTITCRASQSISSYLNWYQQKPGKAPKLLIYGASVLQSGVPSRFSGSGSGTDFTLTISSLQPEDFATYYCQQRVRSPATFGQGTKVEIKRGGSSRSSSSGGGGSGGGGMEVQLLESGGGLVQPGGSLRLSCAASGFAFSSYAMSWVRQAPGKGLEWVSNISGRGAKTTYADSVKGRFTISRDNSKNTLYLQMNSLRAEDTAVYYCAKTYLVFDYWGQGTLVTVSSGGGGSGGGGSGGGGSTDIQMTQSPSSLSASVGDRVTITCRASQSISSYLNWYQQKPGKAPKLLIYRASSLQSGVPSRFSGSGSGTDFTLTISSLQPEDFATYYCQQISATPTTFGQGTKVEIKRAAHHHHHH

SEQ ID NO: 7 - DNA, 3084 bp, Artificial Sequence
  Homo sapien combined with __________
  misc_feature (1)..(6): EcoRI sequence
  misc_feature (13)..(15): Start Codon
  misc_feature (364)..(408): 15-mer linker sequence
  misc_feature (736)..(789): 18-mer linker sequence
  misc_feature (1140)..(1185): 15-mer linker sequence
  misc_feature (1519)..(1551): HIV1 p24 linker
  misc_feature (1903)..(1947): 15-mer linker sequence
  misc_feature (2275)..(2328): 18-mer linker sequence
  misc_feature (2680)..(2724): 15-mer linker sequence
  misc_feature (3058)..(3075): Histidine Tag
  misc_feature (3076)..(3078): Stop Codon
  misc_feature (3079)..(3084): EcoRI sequence
gaattcgccaccatggaggtgcagctgttggagtctgggggaggcttggtacagcctggggggtccctgagactctcctgtgcagcctctggattcacctttagcagctatgccatgagctgggtccgccaggctccagggaaggggctggagtgggtctcagtgattaatgttcctggtaatcgtacatcgtacgcagactccgtgaagggccggttcaccatctccagagacaattccaagaacacgctgtatctgcaaatgaacagcctgagagccgaggacacggccgtatattactgtgcgaaaaagacgcgtcggtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccgttttgcaaagtggggtcccatcaaggttcagtggcagtggatctgggacagatttcactctcaccatcagcagtctgcaacctgaagattttgcaacttactactgtcaacagcgtgttcgttctcctgctacgttcggccaagggaccaaggtggaaatcaaacggggtggttcctctagatcttcctcctctggtggcggtggctcgggcggtggtgggatggaggtgcagctgttggagtctgggggaggcttggtacagcctggggggtccctgagactctcctgtgcagcctctggattcgcctttagcagctatgccatgagctgggtccgccaggctccagggaaggggctggagtgggtctcaaatatttctgggcggggtgctaagacaacgtacgcagactccgtgaagggccggttcaccatctccagagacaattccaagaacacgctgtatctgcaaatgaacagcctgagagccgaggacacggccgtatattactgtgcgaaaacgtatcttgtgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatcgtgcatcctctttgcaaagtggggtcccatcaaggttcagtggcagtggatctgggacagatttcactctcaccatcagcagtctgcaacctgaagattttgcaacttactactgtcaacagatttcggctacgcctactacgttcggccaagggaccaaggtggaaatcaaacgggcggccggagccaccccacaagatttaaacaccatgctaatggaggtgcagctgttggagtctgggggaggcttggtacagcctggggggtccctgagactctcctgtgcagcctctggattcacctttagcagctatgccatgagctgggtccgccaggctccagggaaggggctggagtgggtctcagtgattaatgttcctggtaatcgtacatcgtacgcagactccgtgaagggccggttcaccatctccagagacaattccaagaacacgctgtatctgcaaatgaacagcctgagagccgaggacacggccgtatattactgtgcgaaaaagacgcgtcggtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccgttttgcaaagtggggtcccatcaaggttcagtggcagtggatctgggacagatttcactctcaccatcagcagtctgcaacctgaagattttgcaacttactactgtcaacagcgtgttcgttctcctgctacgttcggccaagggaccaaggtggaaatcaaacggggtggttcctctagatcttcctcctctggtggcggtggctcgggcggtggtgggatggaggtgcagctgttggagtctgggggaggcttggtacagcctggggggtccctgagactctcctgtgcagcctctggattcgcctttagcagctatgccatgagctgggtccgccaggctccagggaaggggctggagtgggtctcaaatatttctgggcggggtgctaagacaacgtacgcagactccgtgaagggccggttcaccatctccagagacaattccaagaacacgctgtatctgcaaatgaacagcctgagagccgaggacacggccgtatattactgtgcgaaaacgtatcttgtgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatcgtgcatcctctttgcaaagtggggtcccatcaaggttcagtggcagtggatctgggacagatttcactctcaccatcagcagtctgcaacctgaagattttgcaacttactactgtcaacagatttcggctacgcctactacgttcggccaagggaccaaggtggaaatcaaacgggcggcccatcatcaccatcaccattaaggtacc

SEQ ID NO: 8 - protein, 1021 aa, Artificial Sequence
  Homo sapien combined with _________
  MISC_FEATURE (118)..(132): 15-mer linker sequence
  MISC_FEATURE (242)..(259): 18-mer linker sequence
  MISC_FEATURE (377)..(391): 15-mer linker sequence
  MISC_FEATURE (503)..(513): HIV1 p24 linker
  MISC_FEATURE (631)..(645): 15-mer linker sequence
  MISC_FEATURE (755)..(772): 18-mer linker sequence
  MISC_FEATURE (890)..(904): 15-mer linker sequence
  MISC_FEATURE (1016)..(1021): Histidine Tag
MEVQLLESGGGLVQPGGSLRLSCAASGFTFSSYAMSWVRQAPGKGLEWVSVINVPGNRTSYADSVKGRFTISRDNSKNTLYLQMNSLRAEDTAVYYCAKKTRRFDYWGQGTLVTVSSGGGGSGGGGSGGGGSTDIQMTQSPSSLSASVGDRVTITCRASQSISSYLNWYQQKPGKAPKLLIYGASVLQSGVPSRFSGSGSGTDFTLTISSLQPEDFATYYCQQRVRSPATFGQGTKVEIKRGGSSRSSSSGGGGSGGGGMEVQLLESGGGLVQPGGSLRLSCAASGFAFSSYAMSWVRQAPGKGLEWVSNISGRGAKTTYADSVKGRFTISRDNSKNTLYLQMNSLRAEDTAVYYCAKTYLVFDYWGQGTLVTVSSGGGGSGGGGSGGGGSTDIQMTQSPSSLSASVGDRVTITCRASQSISSYLNWYQQKPGKAPKLLIYRASSLQSGVPSRFSGSGSGTDFTLTISSLQPEDFATYYCQQISATPTTFGQGTKVEIKRAAGATPQDLNTMLMEVQLLESGGGLVQPGGSLRLSCAASGFTFSSYAMSWVRQAPGKGLEWVSVINVPGNRTSYADSVKGRFTISRDNSKNTLYLQMNSLRAEDTAVYYCAKKTRRFDYWGQGTLVTVSSGGGGSGGGGSGGGGSTDIQMTQSPSSLSASVGDRVTITCRASQSISSYLNWYQQKPGKAPKLLIYGASVLQSGVPSRFSGSGSGTDFTLTISSLQPEDFATYYCQQRVRSPATFGQGTKVEIKRGGSSRSSSSGGGGSGGGGMEVQLLESGGGLVQPGGSLRLSCAASGFAFSSYAMSWVRQAPGKGLEWVSNISGRGAKTTYADSVKGRFTISRDNSKNTLYLQMNSLRAEDTAVYYCAKTYLVFDYWGQGTLVTVSSGGGGSGGGGSGGGGSTDIQMTQSPSSLSASVGDRVTITCRASQSISSYLNWYQQKPGKAPKLLIYRASSLQSGVPSRFSGSGSGTDFTLTISSLQPEDFATYYCQQISATPTTFGQGTKVEIKRAAHHHHHH

SEQ ID NO: 9 - DNA, 15 bp, Homo sapiens
agctatgccatgagc

SEQ ID NO: 10 - protein, 5 aa, Homo sapiens
SYAMS

SEQ ID NO: 11 - DNA, 60 bp, Homo sapiens
gtgattaatgttcctggtaatcgtacatcgtacgcagactccgtgaagggccggttcacc

SEQ ID NO: 12 - protein, 20 aa, Homo sapiens
VINVPGNRTSYADSVKGRFT

SEQ ID NO: 13 - DNA, 60 bp, Homo sapiens
aatatttctgggcggggtgctaagacaacgtacgcagactccgtgaagggccggttcacc

SEQ ID NO: 14 - protein, 20 aa, Homo sapiens
NISGRGAKTTYADSVKGRFT

SEQ ID NO: 15 - DNA, 21 bp, Homo sapiens
aagacgcgtcggtttgactac

SEQ ID NO: 16 - protein, 7 aa, Homo sapiens
KTRRFDY

SEQ ID NO: 17 - DNA, 21 bp, Homo sapiens
acgtatcttgtgtttgactac

SEQ ID NO: 18 - protein, 7 aa, Homo sapiens
TYLVFDY

SEQ ID NO: 19 - DNA, 33 bp, Homo sapiens
cgggcaagtcagagcattagcagctatttaaat

SEQ ID NO: 20 - protein, 11 aa, Homo sapiens
RASQSISSYLN

SEQ ID NO: 21 - DNA, 21 bp, Homo sapiens
ggtgcatccgttttgcaaagt

SEQ ID NO: 22 - protein, 7 aa, Homo sapiens
GASVLQS

SEQ ID NO: 23 - DNA, 24 bp, Homo sapiens
cgtgcatcctctttgcaaagtggg

SEQ ID NO: 24 - protein, 8 aa, Homo sapiens
RASSLQSG

SEQ ID NO: 25 - DNA, 21 bp, Homo sapiens
cgtgttcgttctcctgctacg

SEQ ID NO: 26 - protein, 7 aa, Homo sapiens
RVRSPAT

SEQ ID NO: 27 - DNA, 21 bp, Homo sapiens
atttcggctacgcctactacg

SEQ ID NO: 28 - protein, 7 aa, Homo sapiens
ISATPTT

SEQ ID NO: 29 - DNA, 6 bp, Homo sapiens
gaattc

SEQ ID NO: 30 - DNA, 3 bp, Homo sapiens
atg

SEQ ID NO: 31 - DNA, 45 bp, Homo sapiens
ggtggaggcggttcaggcggaggtggcagcggcggtggcgggtcg

SEQ ID NO: 32 - protein, 15 aa, Homo sapiens
GGGGSGGGGSGGGGS

SEQ ID NO: 33 - DNA, 18 bp, Homo sapiens
catcatcaccatcaccat

SEQ ID NO: 34 - protein, 6 aa, Homo sapiens
HHHHHH

SEQ ID NO: 35 - DNA, 3 bp, Homo sapiens
taa

SEQ ID NO: 36 - DNA, 6 bp, Homo sapiens
ggtacc

SEQ ID NO: 37 - DNA, 54 bp, Homo sapiens
ggtggttcctctagatcttcctcctctggtggcggtggctcgggcggtggtggg

SEQ ID NO: 38 - protein, 18 aa, Homo sapiens
GGSSRSSSSGGGGSGGGG

SEQ ID NO: 39 - DNA, 33 bp, Homo sapiens
ggagccaccccacaagatttaaacaccatgcta

SEQ ID NO: 40 - protein, 11 aa, Homo sapiens
GATPQDLNTML